(19) United States
(12) Patent Application Publication
(10) Pub. No.: US 2024/0003879 A1
(43) Pub. Date: Jan. 4, 2024

(54) METHODS FOR DIAGNOSIS AND MONITORING OF TOXIC EPIDERMAL NECROLYSIS

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); ECOLE NORMALE SUPÉRIEURE DE LYON, Lyon (FR); HOSPICES CIVILS DE LYON, Lyon (FR); UNIVERSITÉ CLAUDE BERNARD - LYON 1, Villeurbanne (FR)

(72) Inventors: Marc VOCANSON, LYON (FR); Axel VILLANI, LYON (FR); Audrey NOSBAUM, LYON (FR); Aurore ROZIERES, LYON (FR)

(21) Appl. No.: 18/038,970

(22) PCT Filed: Nov. 26, 2021
(86) PCT No.: PCT/EP2021/083093

§ 371 Date: May 25, 2023

(30) Foreign Application Priority Data
Nov. 27, 2020 (EP) 20306460.5

(51) Int. Cl. G01N 33/569; C07K 16/28; C12N 15/113

(52) U.S. Cl. CPC G01N 33/56972; C07K 16/2896; C12N 15/1138; G01N 2333/70517; G01N 2333/70596; C07K 2317/24

(57) ABSTRACT

In the present invention, inventors investigate the representation of T cell subsets in Toxic epidermal necrolysis (TEN) a life-threatening cutaneous adverse drug reaction (cADR), characterized by massive epidermal necrosis. To better understand why skin symptoms are so severe in TEN disease, inventors conducted a prospective immunophenotyping study on skin samples and blood from 18 TEN patients, using mass cytometry and next generation TCR sequencing. Deep sequencing of the T cell receptor CDR3 repertoire revealed massive expansion of unique CDR3 clonotypes in blister cells. Over-represented clonotypes were mainly effector memory CD8+CD45RA−CCR7− T cells, and expressed high levels of cytotoxic (Granulysin and Granzymes A & B) and activation (CD38) markers. Thus present invention relates to non-invasive, specific and rapid methods for diagnostic and monitoring Toxic Epidermal Necrolysis. More specifically present invention relates to methods for diagnosis and/or monitoring of Toxic Epidermal Necrolysis through detection of a specific population of T lymphocytes in a subject. The present invention also relates to a method of preventing or treating a Toxic Epidermal Necrolysis in a subject in need thereof.

### FIELD OF THE INVENTION

The present invention relates to methods and kits for diagnostic and monitoring the Toxic Epidermal Necrolysis (TEN). More specifically present invention relates to methods for diagnosis of the Toxic Epidermal Necrolysis through detection of a specific population of CD8+ T cells in a patient. The present invention also relates to a method of preventing or treating a Toxic Epidermal Necrolysis in a subject in need thereof.

### BACKGROUND OF THE INVENTION

Toxic epidermal necrolysis (TEN) is characterized as a rapidly progressing blistering skin rash accompanied by an important mucosal involvement and skin detachment. Hence, TEN is associated with an important mortality rate of approximately 25-40%, and nearly constant and invalidating sequelae (blindness, respiratory disturbance . . . ), which are responsible for profound loss of quality of life in surviving patients (1) (2) (3).

The etiopathogenesis of TEN, like other cutaneous adverse drug reactions (cADRs), involves the activation of drug-specific T cells, which have been isolated and cloned from the blood and the skin lesions of TEN patients (4) (5) (6) (7). Similarly to chemical allergens, the majorities of the drugs responsible for TEN are protein-reactive, and generate new drug-peptide epitopes which trigger an hypersensitivity/allergic reaction (8) (9) (10). Of note, recent works suggest that T cell stimulation could also be consecutive to a direct and non-covalent interaction of the drug with the T Cell Receptor (TCR), or the major histocompatibility complex (MHC)-binding groove (a process referred to as “p-i concept”) (11), as well as via the presentation of an altered repertoire of self-peptides (12) (13).

Although it remains only partially understood, the current paradigm for TEN onset states that, once they have been primed in lymphoid organs, drug-specific cytotoxic CD8+ T cells (CTLs) are recruited at the dermo-epidermal junction where they kill keratinocytes presenting drug epitopes at their surface, through mechanisms involving perforin/granzyme B and MHC class I-restricted pathways (6) (10). To explain extensive blister formation and subsequent skin detachment, several investigators have reported that specific T cells produce massive amounts of soluble mediators like Granulysin (14), interferon-gamma (IFN-γ) or tumor necrosis factor-alpha (TNF-α), that further amplify and extend keratinocyte cell death. IFN-γ and TNF-α promote Fas-L expression on keratinocytes, followed by cell-cell suicide (via Fas-FasL presentation), which may explain the disseminated epidermal apoptosis in some patients (15). Alternatively, other works have suggested that natural killer (NK) cells and inflammatory monocytes exert an additional contribution to epidermal necrolysis, notably via Granulysin-, TWEAK (CD255)- , TRAIL (CD253)- or Annexin A1-dependent mechanisms (16) (17) (18).

These immunological features are now well established, including the skin infiltration by CTLs (19). Yet, most of them were also detected in patients suffering from less severe cADRs, such as maculopapular exanthema (MPE) (20) (21). MPE patients harbour limited spots of epidermal apoptosis/necrolysis (22) (23), but no blisters, and fast healing upon drug discontinuation. Hence, to date, it is still largely unknown why some patients, who sometimes take the same drugs (24) (25), develop a severe and life-threatening disease (TEN) or a mild reaction (MPE). The fact that drug-specific CTLs are involved in diverse types of cADRs questions whether their number, their functions or their activation parameters (i.e. epitope number and persistence, regulatory mechanisms) are peculiar/specific to TEN disease. Moreover, the differential recruitment of unconventional cytotoxic leucocytes could also precipitate the severity of this disease.

Accordingly, there remains an unmet need in the art for specific and more rapid diagnostic test for Toxic epidermal necrolysis, reflecting directly the dysfunction of immune process.

To gain further insight on TEN pathogenesis inventors conducted, here, a comprehensive immunophenotyping study to characterize the immune cells infiltrating the skin or circulating in the blood of patients suffering from TEN or MPE, at time of disease diagnosis. Their results revealed a dramatic clonal expansion of polycytotoxic CD8+ T cells in the blood and skin of TEN patients, which may explain final clinical severity.

The inventors therefore set up a prognostic and monitoring method of the Toxic Epidermal Necrolysis disease that allows to directly reflect the immunological status of the patient.

### SUMMARY OF THE INVENTION

A first object of the present invention relates to an in vitro method for assessing a subject's risk of having or developing Toxic Epidermal Necrolysis, comprising the steps of i) determining in a sample obtained from the subject the level of T lymphocytes having cell surface expression of CD8+CD45RA-CCR7-CD38+ markers, ii) comparing the level determined in step i) with a reference value and iii) concluding when the level of T lymphocytes having cell surface expression of CD8+CD45RA−CCR7−CD38+ markers determined at step i) is higher than the reference value is predictive of a high risk of having or developing Toxic Epidermal Necrolysis.

An additional object of the invention relates to an in vitro method for monitoring a Toxic Epidermal Necrolysis comprising the steps of i) determining the level of a population of T lymphocytes having cell surface expression of CD8+CD45RA−CCR7−CD38+ markers in a sample obtained from the subject at a first specific time of the disease, ii) determining the level of a population of T Lymphocytes having cell surface expression of CD8+CD45RA−CCR7−CD38+ markers in a sample obtained from the subject at a second specific time of the disease, iii) comparing the level determined at step i) with the level determined at step ii) and iv) concluding that the disease has evolved in worse manner when the level determined at step ii) is higher than the level determined at step i).

An additional object of the invention relates to an in vitro method for monitoring the treatment of Toxic Epidermal Necrolysis comprising the steps of i) determining the level of a population of T lymphocytes having cell surface expression of CD8+CD45RA−CCR7−CD38+ in a sample obtained from the subject before the treatment, ii) determining the level of a population of T lymphocytes having cell surface expression of CD8+CD45RA−CCR7−CD38+ markers in a sample obtained from the subject after the treatment”, iii) comparing the level determined at step i) with the level determined at step ii) and iv) concluding that the treatment is efficient when the level determined at step ii) is lower than the level determined at step i).

In a particular embodiment, the sample obtained from the subject, is selected from the list consisting of a blister, a skin or blood sample.

In a particular embodiment regarding the method for assessing subject's risk and monitoring (the disease or the treatment) of the Toxic Epidermal, when the sample is a skin blister, a skin biopsy or blood sample, the level of the population of T lymphocytes having cell surface expression of CD8+CD45RA−CCR7−CD38+ is determined by clonal expansion of said population.

Another object of the invention relates to a CD38 inhibitor for use in the prevention or the treatment of a Toxic Epidermal Necrolysis in a subject in need thereof.

### DETAILED DESCRIPTION OF THE INVENTION

In the present invention, inventors investigated the representation of T cell subsets in Toxic epidermal necrolysis (TEN), a life-threatening cutaneous adverse drug reaction (cADR), characterized by massive epidermal necrosis. Diverse studies have reported that TEN onset correlates with a robust skin infiltration by cytotoxic lymphocytes (T, NK cells) and inflammatory monocytes. To better understand why skin symptoms are so severe in TEN disease, inventors conducted a prospective immunophenotyping study on skin samples and blood from 18 TEN patients, using mass cytometry and next generation TCR sequencing. Inventors confirmed that cytotoxic CD8+ T cells (CTLs) constitute the main leucocyte subset found in TEN blisters, at the acute phase, while the inventors failed to repeatedly detect unconventional lymphocytes such as NKT, MAIT, NK or gamma-delta T cells. Strikingly, deep sequencing of the T cell receptor CDR3 repertoire revealed massive expansion of unique CDR3 clonotypes in blister cells. Over-represented clonotypes were mainly effector memory CD8+CD45RA−CCR7− T cells, and expressed high levels of cytotoxic (Granulysin and Granzymes A & B) and activation (CD38) markers. By transfecting α and β chains of the expanded clonotypes into immortalized T cells, the inventors confirmed in some patients that those cells were drug-specific. Collectively, the inventors suggest that the quantity (clonal expansions) and quality (cytotoxic phenotype) of skin-recruited CTLs condition the clinical presentation of cADRs. Importantly, they open major opportunities for the development of new prognostic avenues in TEN. This biomarker set may be used as prognosis tool in combination with clinical scores. These results thus set-up the basis for the development of a rapid functional specific test for critical form of TEN.

Diagnostic Methods According to the Invention

The present invention relates to an in vitro method for assessing a subject's risk of having or developing Toxic Epidermal Necrolysis, comprising the steps of i) determining in a sample obtained from the subject the level of T lymphocytes having cell surface expression of CD8+CD45RA−CCR7−CD38+ markers, ii) comparing the level determined in step i) with a reference value and iii) concluding when the level of T lymphocytes having cell surface expression of CD8+CD45RA−CCR7−CD38+ markers determined at step i) is higher than the reference value is predictive of a high risk of having or developing Toxic Epidermal Necrolysis.

In another term, the present invention relates to an in vitro diagnostic method of having or developing Toxic Epidermal Necrolysis in a subject, comprising the steps of i) determining in a sample obtained from the subject the level of T lymphocytes having cell surface expression of CD8+CD45RA−CCR7−CD38+ markers ii) comparing the level determined in step i) with a reference value and iii) concluding when the level of T lymphocytes having cell surface expression of CD8+CD45RA−CCR7−CD38+ markers determined at step i) is higher than the reference value is predictive of having or developing Toxic Epidermal Necrolysis.

In the context of the present invention the “diagnosis” is associated with level of T lymphocytes having cell surface expression of CD8+CD45RA−CCR7−CD38+ markers which in turn may be a risk for developing Toxic Epidermal Necrolysis disease.

The term “subject” as used herein refers to a mammalian, such as a rodent (e.g. a mouse or a rat), a feline, a canine or a primate. In a preferred embodiment, said subject is a human subject. The subject according to the invention can be a healthy subject or a subject suffering from a given cutaneous adverse drug reactions (cADRs) disease such as Maculo-Papular Exanthema related to drug (MPE) or Toxic Epidermal Necrolysis (TEN).

As used herein, the term “cutaneous adverse drug reactions” (or “cADRs”) (The terms “cARDs” and “cutaneous adverse drug reactions” are used herein interchangeably) are a group of potentially lethal adverse drug reactions that involve the skin and mucous membranes of various body openings such as the eyes, ears, and inside the nose, mouth, and lips. In more severe cases, cADRs could also involve serious damage to internal organs. cADRs include these different syndromes: Drug reaction with eosinophilia and systemic symptoms (i.e. DRESS syndrome, also termed Drug-induced hypersensitivity syndrome [DIHS]); Stevens-Johnson syndrome (SJS); Toxic epidermal necrolysis (TEN), Stevens-Johnson/toxic epidermal necrolysis overlap syndrome (SJS/TEN); acute generalized exanthematous pustulosis (AGEP), Maculo-Papular Exanthema related to drug (MPE), Fixed Drug Eruption (FDE), Symmetrical Drug Related Intertriginous and Flexural Exanthema (SDRIFE). These disorders have similar pathophysiologies, i.e. disease-causing mechanisms, for which new strategies are in use or development to identify individuals predisposed to develop the cADRs—inducing effects of specific drugs and thereby avoid treatment with them (Adler N R, et al (2017) The British Journal of Dermatology. 177 (5): 1234-1247).

Adverse drug reactions are major therapeutic problems estimated to afflict up to 20% of inpatients and 25% of outpatients. About 90% of these delayed adverse reactions take the form of benign morbilliform rash hypersensitivity drug reactions called maculo-papular exanthema (MPE). cADRS are delayed-hypersentivity reaction called Type IV hypersensitivity reaction of the innate immune system initiated by lymphocytes of the T cell type and mediated by various types of leukocytes and cytokines (Garon S L et al (2017). British Journal of Clinical Pharmacology. 83 (9): 1896-1911).

cADRs are here considered as a group focusing on the similarities and differences in their pathophysiologies, clinical presentations, instigating drugs, and recommendations for drug avoidance.

The mains drugs knows to inducing cADRs are for instance but not limited to: anti-epileptics (Stern R S; N Engl J Med 2012; 366:2492-501), antibiotics (such as Vancomycin, Penicillin, Cephalosporin, Tetracycline, Fluoroquinolone, Sulfonamide, Cotrimoxazole, Carbapenem, . . . ) (Wolfson A R et al Allergy Clin Immunol Pract Month 2018), antiretroviral drugs, Immune Checkpoint inhibitors (ICP) (see Nagash et al. Journal for ImmunoTherapy of Cancer (2019) 7:4), proton pump inhibitors, anticonvulsants (such as phenobarbital, carbamazepine, phenytoin, lamotrigine, and sodium valproate) and Allopurinol, (used to decrease high blood uric acid levels) (see also the review V. Descamps, et al Joint Bone Spine 81 (2014) 15-21) The term “subject suspected of having cADRs” refers to a subject that presents one or more symptoms indicative of cADRs (e.g., pain, skins or and mucous membranes lesions associated with drugs administration), or that is screened for cADRs (e.g., during a physical examination). Alternatively or additionally, a subject suspected of having cADRs may have one or more risk factors (e.g., age, sex, family history, etc). The term encompasses subjects that have not been tested for cADRs as well as subjects that have received an initial diagnosis.

As used herein, the term “TEN” or “Toxic Epidermal Necrolysis” refers to a life-threatening cutaneous adverse drug reaction (cADR), characterized by massive epidermal necrosis. Toxic epidermal necrolysis (TEN) is characterized as a rapidly progressing blistering eruption accompanied by an important mucosal involvement and skin detachment. Hence, TEN is associated with an important mortality rate of approximately 25-40%, and nearly constant and invalidating sequelae (blindness, respiratory disturbance . . . ), which are responsible for profound loss of quality of life in surviving patients (1) (2) (3).

The etiopathogenesis of TEN, like other cutaneous adverse drug reactions (cADRs), involves the activation of drug-specific T cells, which have been isolated and cloned from the blood and the skin lesions of TEN patients (4) (5) (6) (7). Similarly to chemical allergens, the majorities of the drugs responsible for TEN are protein-reactive, and generate new drug-peptide epitopes, which trigger an hypersensitivity/allergic reaction (8) (9) (10). Of note, recent works suggest that T cell stimulation could also be consecutive to a direct and non-covalent interaction of the drug with the T Cell Receptor (TCR), or the major histocompatibility complex (MHC)-binding groove (a process referred to as “p-i concept”) (11), as well as via the presentation of an altered repertoire of self-peptides (12) (13).

In particular embodiments, the subject of the present invention suffers from TEN and/or have been previously diagnosed with cADRs.

As used herein, the term “sample” or “biological sample” as used herein refers to any biological sample of a subject and can include, by way of example and not limitation, bodily fluids and/or tissue extracts such as homogenates or solubilized tissue obtained from a subject. Tissue extracts are obtained routinely from tissue biopsy. In a particular embodiment regarding the prognostic method of the critical form of the Toxic Epidermal Necrolysis according to the invention, the biological sample is a body fluid sample (such as blister fluid, blood or immune primary cell) or skin biopsy of said subject.

In particular embodiments, the fluid sample is a blood sample. The term “blood sample” means a whole blood sample obtained from a subject (e.g. an individual for which it is interesting to determine whether a population of T lymphocytes can be identified).

In particular embodiments, the fluid sample is a blister sample. The term “blister” describes a bubble of fluid under the skin. The clear, watery liquid inside a blister is called the blister fluid. It leaks in from neighboring tissues as a reaction to inflamed skin. If the blister remains unopened, liquid and immune primary cells can be collected. Small blisters are called vesicles. Those larger than half an inch are called bullae.

The term “immune primary cell” has its general meaning in the art and is intended to describe a population of white blood cells directly obtained from a subject.

In the context of the present invention immune primary cell is selected from the group consisting of PBMC, WBC, T Lymphocytes.

The term “PBMC” or “peripheral blood mononuclear cells” or “unfractionated PBMC”, as used herein, refers to whole PBMC, i.e. to a population of white blood cells having a round nucleus, which has not been enriched for a given sub-population (which contain T lymphocytes, (also referred to as T cells), B cells, natural killer (NK) cells, NK T cells and DC precursors). A PBMC sample according to the invention therefore contains different lymphocytes (B cells, T cells, NK cells, NKT cells). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer, which will preferentially lyse red blood cells. Such procedures are known to the expert in the art.

The term “WBC” or “White Blood Cells”, as used herein, also refers to leukocytes population, are the cells of the immune system. All white blood cells are produced and derived from multipotent cells in the bone marrow known as hematopoietic stem cells. Leukocytes are found throughout the body, including the blood and lymphatic system. Typically, WBC or some cells among WBC can be extracted from whole blood by using i) immunomagnetic separation procedures, ii) percoll or ficoll density gradient centrifugation, iii) cell sorting using flow cytometer (FACS). Additionally, WBC can be extracted from whole blood using a hypotonic lysis buffer, which will preferentially lyse red blood cells. Such procedures are known to the expert in the art.

In some embodiments, the fluid sample is a sample of purified T Lymphocytes in suspension. Typically, the sample of T lymphocytes is prepared by immunomagnetic separation methods preformed on a PBMC or WBC sample. For example, T Lymphocytes are isolated by using antibodies for T lymphocyte-associated cell surface markers, such as CD8 and CD38. Commercial kits, e.g. Direct Human T Lymphocyte Isolation Kit kits (Immunomagnetic positive selection from whole blood kit) using anti-CD8 labelled antibodies (#19663 from Stem cells technologies) are available.

The term “T cells” (also called “T lymphocytes”) represent an important component of the immune system that plays a central role in cell-mediated immunity. T cells are known as conventional lymphocytes as they recognize a specific antigen with their TCR (T Cell Receptor for the antigen) with presentation or restriction by molecules of the major histocompatibility complex. There are several subsets of T cells each having a distinct function such as CD8+ T cells, CD4+ T cells, regulatory T-cells.

In the context of the present invention, the T cell is CD8+ T cell. The term “CD8+ T cells” (also called Cytotoxic T cells or TC cells, CTLs, T-killer cells or killer T cells) is used to describe T lymphocytes, which express the CD8 glycoprotein at their surface and when activated by host cells presenting specific antigens (APCs) via MHC I, are able to destroy infected cells and tumor cells, presenting the same antigens on their surface. Naïve CD8+ T cells have numerous acknowledged biomarkers known in the art. These include in human CD45RA+CCR7+HLA−DR−CD8+ and the TCR chain is formed of an alpha chain (a) and a beta chain (0).

As used herein, the term “CD8”, also known as cluster of differentiation 8 has its general meaning in the art refers to a transmembrane glycoprotein that serves as a co-receptor for the T-cell receptor (TCR). Along with the TCR, the CD8 co-receptor plays a role in T cell signaling and aiding with cytotoxic T cell antigen interactions. Like the TCR, CD8 binds to a major histocompatibility complex (MHC) molecule, but is specific for the MHC class I protein. There are two isoforms of the protein, alpha and beta, each encoded by a different gene (Gene ID CD8A: 825/Gene ID CD8B: 926). In humans, both genes are located on chromosome 2 in position 2p12.

As used herein, the term “CD45RA” (Cluster of Differentiation 45) also known as Protein tyrosine phosphatase, receptor type, C (PTPRC) has its general meaning in the art refers to an enzyme that, in humans, is encoded by the PTPRC gene (gene ID 5788)). The protein product of this gene, best known as CD45, is a member of the protein tyrosine phosphatase (PTP) family. PTPs are signaling molecules that regulate a variety of cellular processes including cell growth, differentiation, mitotic cycle, and oncogenic transformation. CD45 contains an extracellular domain, a single transmembrane segment, and two tandem intracytoplasmic catalytic domains, and thus belongs to the receptor type PTP family.

CD45 is a type I transmembrane protein that is present in various isoforms on all differentiated hematopoietic cells (except erythrocytes and plasma cells). CD45 has been shown to be an essential regulator of T- and B-cell antigen receptor signaling. It functions through either direct interaction with components of the antigen receptor complexes via its extracellular domain (a form of co-stimulation), or by activating various Src family kinases required for the antigen receptor signaling via its cytoplasmic domain. CD45 also suppresses JAK kinases, and so functions as a negative regulator of cytokine receptor signaling. Many alternatively spliced transcripts variants of this gene, which encode distinct isoforms, have been reported. Antibodies against the different isoforms of CD45 are used in routine immunohistochemistry to differentiate between immune cell types, as well as to differentiate between histological sections from lymphomas and carcinomas.

The expression of CD45RA on T cell serves to identify distinct subset. Naive T lymphocytes are typically positive for CD45RA, which includes only the A protein region. Activated and memory T lymphocytes express CD45RO, the shortest CD45 isoform, which lacks all three of the A, B, and C regions. This shortest isoform facilitates T cell activation

In the context of the method of the invention “CD45RA-means that the cell surface marker is not expressed on T lymphocytes (or not detected when contacted for instance with a labeled CD45RA antibody).

The term “CCR7” or “C—C chemokine receptor type 7” has its general meaning in the art refers to an protein that, in humans, is encoded by the CCR7 gene (gene ID 1236)). Two ligands have been identified for this receptor: the chemokines (C—C motif) ligand 19 (CCL19/ELC) and (C—C motif) ligand 21 (CCL21). CCR7 has also recently been designated CD197 (cluster of differentiation 197).

The protein receptor CCR7 encoded by this gene is a member of the G protein-coupled receptor family. This receptor was identified as a gene induced by the Epstein-Barr virus (EBV), and is thought to be a mediator of EBV effects on B lymphocytes. This receptor is expressed in various lymphoid tissues and activates B and T lymphocytes. CCR7 has been shown to stimulate dendritic cell maturation. CCR7 is also involved in homing of T cells to various secondary lymphoid organs such as lymph nodes and the spleen as well as trafficking of T cells within the spleen. Activation of dendritic cells in peripheral tissues induces CCR7 expression on the cell's surface, which recognize CCL19 and CCL21 produced in the lymph node and increases dendritic cell expression of co-stimulation molecules (B7), and MHC class I or MHC class II.

In the context of the method of the invention “CCR7-” means that the cell surface marker is not expressed on T lymphocytes (or not detected when contacted for instance with a labeled CCR7 antibody).

As used herein, the term “CD38” also knows as Cluster of Differentiation 38 or “cyclic ADP ribose hydrolase” (ADPRC1) refers to a transmembrane glycoprotein (Orciani M, et al (2008). Journal of Cellular Biochemistry. 105 (3): 905-12) found on the surface of many immune cells (lymphocytes), including CD4+, CD8+, B lymphocytes and natural killer cells. CD38 also functions in cell adhesion, signal transduction and calcium signaling. In humans, the CD38 protein is encoded by the CD38 gene (Gene ID: 952) which is located on chromosome 4 (Jackson D G et al (1990). Journal of Immunology. 144 (7): 2811-5).

CD38 can function either as a receptor or as an enzyme (Nooka A K, et al (2019). Cancer. 125 (14): 2364-2382). As a receptor, CD38 can attach to CD31 on the surface of T cells, thereby activating those cells to produce a variety of cytokines (Nooka A K, et al (2019)). CD38 is a multifunctional ectoenzyme that catalyzes the synthesis and hydrolysis of cyclic ADP-ribose (cADPR) from NAD+ to ADP-ribose in addition to synthesis of NAADP from NADP+ (Chini E N, et al (2002). The Biochemical Journal. 362 (Pt 1): 125-30). These reaction products are essential for the regulation of intracellular Ca2+ (Malavasi F, et al (2008). Physiological Reviews. 88 (3): 841-86). CD38 occurs not only as an ectoezyme on cell outer surfaces, but also occurs on the inner surface of cell membranes, facing the cytosol performing the same enzymatic functions (Lee H C, et al (2019). Journal of Biological Chemistry. 294 (52): 19831-19843). CD38 is used as a prognostic marker for patients with chronic lymphocytic leukemia.

One example of CD38+ human amino acid sequence (UniProtKB-P28907) is provided in SEQ ID NO: 1 (Transcript variant 1 NCBI Reference Sequence: NP_001766). One example of nucleotide sequence encoding wild-type CD38+ is provided in SEQ ID NO: 2 (transcript variant 1 NCBI Reference Sequence: NM_001775). Alternative splicing results in multiple transcript variants (NCBI web site “Entrez Gene: CD38 molecule”).

Of course variant sequences of the CD38+ may be used in the context of the present invention (as biomarker or therapeutic target), those including but not limited to functional homologues, paralogues or orthologues, transcript variants of such sequences.

Standard methods for detecting the expression of a specific surface marker such as CD8 or CD38 at cell surface (e.g. T lymphocyte surface) are well known in the art. Typically, the step consisting of detecting the surface expression of a surface marker (e.g. CD8 or CD38 1) or detecting the absence of the surface expression of a surface marker ((e;g. CD45RA or CCR7) may consist in using at least one differential binding partner directed against the surface marker, wherein said cells are bound by said binding partners to said surface marker.

As used herein, the term “binding partner directed against the surface marker” refers to any molecule (natural or not) that is able to bind the surface marker with high affinity. The binding partners may be antibodies that may be polyclonal or monoclonal, preferably monoclonal antibodies. In another embodiment, the binding partners may be a set of aptamers.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally; the human B-cell hybridoma technique; and the EBV-hybridoma technique.

For example, the binding partner of CD8 of the invention is the anti-human CD8 antibody available from Biolegend (CD8 Monoclonal Antibody (clone SK1)).

For example, the binding partner of CD38 of the invention is the anti-human CD38 antibody available from Biolegend (PE anti-human CD38 Antibody (clone HIT2 or clone 90) or from Miltenyi Biotech (Anti-CD38 Antibody, anti-human, REAfinity (clone REA671).

The binding partners of the invention such as antibodies or aptamers may be labelled with a detectable molecule or substance, such as preferentially a fluorescent molecule, or a radioactive molecule or any others labels known in the art. Labels are known in the art that generally provide (either directly or indirectly) a signal.

As used herein, the term “labelled”, with regard to the antibody or aptamer, is intended to encompass direct labelling of the antibody or aptamer by coupling (i.e., physically linking) a detectable substance, such as a fluorophore [e.g. fluorescein isothiocyanate (FITC) or phycoerythrin (PE) or Indocyanine (Cy5)]) or radioactive molecule or a non-radioactive heavy metals isotopes to the antibody or aptamer, as well as indirect labelling of the probe or antibody by reactivity with a detectable substance. An antibody or aptamer of the invention may be labelled with a radioactive molecule by any method known in the art. More particularly, the antibodies are already conjugated to a fluorophore (e.g. FITC-conjugated and/or PE-conjugated).

The aforementioned assays may involve the binding of the binding partners (ie. antibodies or aptamers) to a solid support. The solid surface could be a microtitration plate coated with the binding partner for the surface marker. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, California). According to the invention, methods of flow cytometry are preferred methods for detecting (presence or absence of) the surface expression of the surface markers (i.e. CD8, CD45RA, CCR7 and CD38). Said methods are well known in the art. For example, fluorescence activated cell sorting (FACS) may be therefore used. Cell sorting protocols using fluorescent labeled antibodies directed against the surface marker (or immunobeads coated with antibody) in combination with antibodies directed against CD8, CD45RA, CCR7 and CD38 coupled with distinct fluorochromes (or immunobeads coated with anti-CD8, anti CD45RA antibodies, anti CCR7 antibodies, a and anti CD38+ antibodies) can allow direct sorting, using cell sorters with the adequate optic configuration.

Such methods comprise contacting a biological sample obtained from the subject to be tested under conditions allowing detection (presence or absence) of CD8, CD45RA, CCR7 and CD38 surface markers. Once the sample from the subject is prepared, the level of TEN biomarkers (“Biomarker”: CD8+CD45RA−CCR7−/CD38+ T cells) may be measured by any known method in the art.

Typically, the high or low level of TEN-associated T cell surface biomarkers (“Biomarker”: CD8+CD45RA−CCR7−/CD38+ T cells) is intended by comparison to a control reference value.

Said reference control values may be determined in regard to the level of biomarker present in blood samples taken from one or more healthy subject(s) or to the cell surface biomarker in a control population.

In one embodiment, the method according to the present invention comprises the step of comparing said level of TEN-associated T lymphocyte biomarkers (“Biomarker”: CD8+CD45RA−CCR7− T cells) to a control reference value wherein a high level of TEN-associated T lymphocyte biomarkers (“Biomarker”: CD8+CD45RA−CCR7− T cells) compared to said control reference value is predictive of a high risk of having a critical form of Toxic Epidermal Necrolysis and a low level of TEN—associated T lymphocyte biomarkers (“Biomarker”: CD8+CD45RA−CCR7− T cells) compared to said control reference value is predictive of a low risk of having or developing a critical form of Toxic Epidermal Necrolysis.

In a specific embodiment when the sample is a skin blister, a skin biopsy or a blood sample, the level of expression of the TEN-associated T lymphocyte biomarker (“Biomarker”: CD8+CD45RA−CCR7−/CD38+ T cells) is detected by clonal expansion.

The term “Clonal Expansion” has its general meaning in the art refers to multiplication or reproduction by cell division of a population of identical cells descended from a single progenitor. In immunology, may refer to the clonal proliferation of cells responsive to a specific antigen as part of an immune response. T cells respond to specific antigen by using their T-cell receptors (TCR), which bind and recognize peptide antigens presented by major histocompatibility complex (MHC) molecules located on the surface of antigen-presenting cells, such as dendritic cells. During an immune response, antigen presentation then results in the activation and expansion of a multitude of T cells with unique TCR(s) specificities.

The term “TCR repertoire diversity” refers to the specificity of T cells for an individual antigenic peptide-MHC complex is primarily determined by the amino-acid sequence in the hypervariable complementarity-determining region 3 (CDR3) of the α- and β-chains of the TCR. During T cell development, each TCR chain is generated through a process called somatic DNA recombination, where noncontiguous variable (V), diversity (D), and joining (J) gene segments encoded within the germline are rearranged to form a unique TCR sequence within an immature T cell. During this process, try, trd, and trj segments are rearranged together to create and encode CDR3, the most variable region of the TCR that interacts with foreign peptide. Rearrangement of multiple V, D and J gene segments, as well as the random insertion and/or deletion of nucleotides at the gene junctions, can theoretically result in up to 1×10¹⁸ unique CDR3 sequences.

Identifying clonal expansion in the context of the present invention may be determined for instance: (i) by analyzing by Flow (or Mass) Cytometry the expression of respective TCR Vβ and/or Vα chains in the T cell population of interest (CD8+CD45RA−CCR7−/CD38+ T cells), and also (ii) by performing high-throughput sequencing (HTS) of the TCR CDR3 regions (the antigen recognition domains) to evaluate sample clonality (see Example Section).

The control reference value may depend on various parameters such as the method used to measure the level of TEN-associated T lymphocyte biomarker Biomarker CD38+(CD8+CD45RA−CCR7−CD38+ T cells) or the gender of the subject.

Typically regarding the reference value using “Biomarker” (CD8+CD45RA−CCR7−CD38+ T cells), as indicated in the Example section (FIG. 4), for a level of T lymphocytes CD8+CD45RA−CCR7−CD38+ using Flow Cytometry approach identify and quantify T lymphocyte population with clonality index (in skin sample), a level of T lymphocyte CD8+CD45RA−CCR7−CD38+ superior to 0.14 (as determined according to the Tukey's rule for the detection of outliers) is predictive of having or a high risk of having or developing Toxic Epidermal Necrolysis and a level of T lymphocyte CD8+CD45RA−CCR7−CD38+ lower than is predictive of not having or at a low risk of having Toxic Epidermal Necrolysis.

Control reference values are easily determinable by the one skilled in the art, by using the same techniques as for determining the level of cell surface biomarker or clonality index in blood samples previously collected from the patient under testing.

A “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the level of T lymphocyte biomarkers (“Biomarker”: CD8+CD45RA−CCR7-CD38+ T cells) with a defined threshold value. In one embodiment of the present invention, the threshold value is derived from the T lymphocyte level (or ratio, or score) determined in a blood sample derived from one or more subjects who are responders (to the method according to the invention). In one embodiment of the present invention, the threshold value may also be derived from T lymphocyte level (or ratio, or score) determined in a blood sample derived from one or more subjects who are non-responders (ie MPE patient). Furthermore, retrospective measurement of the activated T lymphocyte level (or ratio, or scores) in properly banked historical subject samples may be used in establishing these threshold values.

Reference values are easily determinable by the one skilled in the art, by using the same techniques as for determining the level of activated T Lymphocytes in fluids samples previously collected from the patient under testing.

“Risk” in the context of the present invention, relates to the probability that an event will occur over a specific time period, as in the conversion to critical form of Toxic Epidermal Necrolysis, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no conversion. Alternative continuous measures, which may be assessed in the context of the present invention, include time to critical form of Toxic Epidermal Necrolysis conversion risk reduction ratios.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition or asymptomatic form of TEN or symptomic form of TEN to a critical form of Toxic Epidermal Necrolysis condition or to one at risk of developing Toxic Epidermal Necrolysis (or a critical form of TEN). Risk evaluation can also comprise prediction of future clinical parameters, traditional laboratory risk factor values, or other indices of Toxic Epidermal Necrolysis, such as cellular population determination in peripheral tissues, in serum or other fluid, either in absolute or relative terms in reference to a previously measured population. The methods of the present invention may be used to make continuous or categorical measurements of the risk of conversion to Toxic Epidermal Necrolysis (or a critical form of TEN), thus diagnosing and defining the risk spectrum of a category of subjects defined as being at risk for Toxic Epidermal Necrolysis (or a critical form of TEN). In the categorical scenario, the invention can be used to discriminate between normal and other subject cohorts at higher risk for Toxic Epidermal Necrolysis (or a critical form of TEN). In other embodiments, the present invention may be used so as to help to discriminate those having TEN from critical form of Toxic Epidermal Necrolysis.

Accordingly, the method of detection of the invention is consequently useful for the in vitro diagnosis of TEN from a biological sample. In particular, the method of detection of the invention is consequently useful for the in vitro diagnosis of TEN from a biological sample.

In a particular embodiment, the method of the present invention, further comprise additional step iv) of determining in said sample the level of Granulysin and/or Granzymes (A & B) mediators produced by T cells having cell surface expression of CD8+CD45RA−CCR7−CD38+ markers, v) comparing the level determined in step iv) with a reference value and vi) concluding when the level of Granulysin and/or Granzymes (A & B) mediators produced by T cells having cell surface expression of CD8+CD45RA−CCR7−CD38+ markers determined at step iv) is higher than the reference value is predictive of a high risk of having or developing Toxic Epidermal Necrolysis.

In a particular embodiment, the method of the present invention, further comprise additional step iv) of determining in said sample the expression level of TCRVβ and/or TCRVα chains in CD8+ T cells, v) comparing the level determined in step iv) with a reference value and vi) concluding when the level of TCR Vβ and Vα chains in CD8+ T cells determined at step iv) is higher than the reference value is predictive of a high risk of having or developing Toxic Epidermal Necrolysis.

In another embodiment this additional step can be performed before the determination of the level of T lymphocytes having cell surface expression of CD8+CD45RA−CCR7−CD38+ markers.

The control reference value may depend on various parameters such as the method used to measure the expression level of TCRVβ and/or TCRVα and expression level of TCRVβ and/or TCRVα may depend on various parameters such as the method used to or the gender of the subject.

Typically regarding the reference value using “Biomarker” (CD8+CD45RA−CCR7−CD38+ T cells), as indicated in the Example section (FIG. 3), a level of TCRVβ and/or TCRVα (in skin, blister or blood sample) in T lymphocytes CD8+CD45RA−CCR7−CD38+ superior to the Tukey's rule for the detection of outliers (75th percentile (Q3)+1.5×inter-quartile range (IQR), by compiling all donor data for each Vβ or Vα chain), is predictive of having or a high risk of having or developing Toxic Epidermal Necrolysis and a level of T lymphocyte CD8+CD45RA−CCR7−CD38+ lower than the Tukey's rule for the detection of outliers (75th percentile (Q3)+1.5×inter-quartile range (IQR), by compiling all donor data for each Vβ or Vα chain), is predictive of not having or at a low risk of having Toxic Epidermal Necrolysis.

Monitoring methods and Management

After the identification of the T cell subset which harbors a polycytotoxic effector memory cell phenotype (“Biomarker”: CD8+CD45RA−CCR7−CD38+ cells,”), inventors highlighted, that CD38+ expressing CD8+CD45RA−/CCR7− T cell subsets (“cytotoxic T cells”) strongly correlated with TEN severity scores (see FIG. 5). Accordingly, inventors provided evidence that this cytotoxic T cell subset may serve as a severity biomarker in TEN for prognosis and monitoring purpose.

Accordingly, “cytotoxic T cells according to the invention, is a population of T lymphocytes having cell surface expression of CD8+CD45RA−CCR7−CD38+.

Accordingly, an additional object of the invention relates to an in vitro method for monitoring a Toxic Epidermal Necrolysis comprising the steps of i) determining the level of a population of T lymphocytes having cell surface expression of CD8+CD45RA−CCR7−CD38+ markers in a sample obtained from the subject at a first specific time of the disease, ii) determining the level of a population of T Lymphocytes having cell surface expression of CD8+CD45RA−CCR7−CD38+ markers in a sample obtained from the subject at a second specific time of the disease, iii) comparing the level determined at step i) with the level determined at step ii) and iv) concluding that the disease has evolved in worse manner when the level determined at step ii) is higher than the level determined at step i).

An additional object of the invention relates to an in vitro method for monitoring the treatment of a Toxic Epidermal Necrolysis comprising the steps of i) determining the level of a population of T Lymphocytes having cell surface expression of CD8+CD45RA−CCR7−CD38+ in a sample obtained from the subject before the treatment, ii) determining the level of a population of T Lymphocytes having cell surface expression of CD8+CD45RA−CCR7−CD38+ markers in a sample obtained from the subject after the treatment”, iii) comparing the level determined at step i) with the level determined at step ii) and iv) concluding that the treatment is efficient when the level determined at step ii) is lower than the level determined at step i).

In particular embodiment, the sample obtained from the subject, is selected from the list consisting of a skin blister, a skin biopsy or a blood sample.

In a specific embodiment when the sample is a skin blister, a skin biopsy or a blood sample, the level of expression of the TEN-associated T lymphocyte biomarker (“Biomarker”: CD8+CD45RA−CCR7−/CD38+ T cells) is detected by clonal expansion

The decrease can be e.g. at least 5%, or at least 10%, or at least 20%, more preferably at least 50% even more preferably at least 100%.

Therapeutic Method

The loss of CD38 function is associated with impaired immune responses, metabolic disturbances. The CD38 protein is a marker of cell activation. It has been connected to HIV infection, leukemias, myelomas (Marlein C R, et al (2019). Cancer Research. 79 (9): 2285-2297) solid tumors, type II diabetes mellitus and bone metabolism. CD38 as been used as a prognostic marker in leukemia (Deaglio S, et al (2001). Leukemia Research. 25 (1): 1-12) and Daratumumab (Darzalex) which targets CD38 has been used in treating multiple myeloma (McKeage K (2016). Drugs. 76 (2): 275-81 and Xia C, et al (2016). Drugs of Today. 52 (10): 551-560).

In the present invention, inventors show that CD38+ expression on T lymphocytes seems to be detrimental for TEN patients as is associated with the secretion of several cytotoxic mediators, such as Granzyme A, Granzyme B and especially Granulysin, and with TEN severity. Furthermore inventors demonstrate that the strength of clonal expansions of CD8+ T cells reached levels (both in skin and blood) that were only described in skin neoplasic disorders, such as cutaneous T cell lymphomas (CTCLs) (32). Additionally, the fact that inventors results can be generalized to patients expressing highly diverse HLA genotypes and reactive to very different drugs (Table 1), thus reinforces the idea that a massive clonal bias is a major immunological hallmark of TEN disease. Finally, as CD38+ is classically associated with T cell activation and/or diapedesis (lymphocyte migration through the capillary barrier in an inflammatory process) in tissues, it should be thus considered as potential target for therapeutic intervention to prevent the (re)activation and the infiltration of the cells that are responsible for this skin pathology.

The inventors show that CD38 is expressed and dysregulated in the effector memory T cell population of the TEN subject. CD38 have a potential role in Toxic Epidermal Necrolysis pathogenesis.

The depletion of human CD38+CD8+ T cells with an anti-CD38 mAb (Daratumumab) was previously evaluated in a murine model of graft versus host versus disease (GVHD). This resulted in the reduction of human CD38+CD8+ T cells levels in blood. The same approach is developed in a TEN preclinical model using reconstituted with human CD38+CD8+ T cells from patients (see example 2).

Accordingly, in an additional aspect the invention relates to a method of preventing or treating a Toxic Epidermal Necrolysis in a patient in need thereof comprising administering to the patient a therapeutically effective amount of a CD38 inhibitor.

In a particular embodiment, the invention relates to a CD38 inhibitor for use in the prevention or the treatment of a Toxic Epidermal Necrolysis in a subject in need thereof.

In a particular embodiment, the invention relates to a CD38 inhibitor for use in the prevention or the treatment of a Toxic Epidermal Necrolysis in a subject in need thereof, wherein the level of a population of T lymphocytes CD8+CD45RA−CCR7−CD38+ obtained from said patient, have been detected by one of the methods (prognostic or monitoring) of the invention.

In its broadest meaning, the term “treating” or “treatment” refers to reversing, alleviating, inhibiting the progress of Toxic Epidermal Necrolysis, preferably inhibiting the severe form of Toxic Epidermal Necrolysis. In particular, “prevention” or “prophylactic treatment” of Toxic Epidermal Necrolysis may refer to the administration of the compounds of the present invention that prevent the symptoms of Toxic Epidermal Necrolysis, in particular the severe form of Toxic Epidermal Necrolysis.

According to the invention, the term “subject” denotes a mammal, such as a rodent, a feline, a canine, or a primate. In some embodiments, the subject is a human. In some embodiments, the subject is an elderly human. Particularly, the subject denotes a human with a pathogen viral infection. Particularly, the subject denotes a human with a Toxic Epidermal Necrolysis. As used herein, the term “subject” encompasses the term “patient”.

As used herein, the term “CD38+ inhibitor” refers to a natural or synthetic compound that has a biological effect to inhibit the activity or the expression of CD38.

The term “inhibitor” as used herein, refers to an agent that is capable of specifically binding and inhibiting signalling through a receptor (or an enzyme) to fully block, as does an inhibitor, or detectably inhibit a response mediated by the receptor (or the enzyme). For example, as used herein the term “CD38+ inhibitor” is a natural or synthetic compound, which binds and inactivates fully or partially CD38+ for initiating or participating to a pathway signaling (such as the cytokine production) and further biological processes. In the context of the invention the CD38+ inhibitor in particular prevents, decreases or suppresses the clonal expansion of the CD8+CD38+ T cells by depleting them. The clonal expansion decrease observed can be by at least about by 20%, 30%, 40%, 50%, 60%, 70%, 80%, or 90%, as compared to the clonal expansion observed in a referenced cell population.

CD38 inhibitory activity may be assessed by various known methods. A control CD38 can be exposed to no antibody or antigen binding molecule, an antibody or antigen binding molecule that specifically binds to another antigen, or an anti-CD38 antibody or antigen binding molecule known not to function as an inhibitor, for example as an inhibitor.

In some embodiment, the CD38 inhibitor inhibits the CD38 actions that exacerbate the effects of clonal expansion of CD8+CD38+ T cells and pro-cytotoxic mediator release (Granulysin and/or Granzymes (A & B)) would be an effective therapeutic option for Toxic Epidermal Necrolysis and its consequences.

By “biological activity” of CD38 is meant inducing pro-cytotoxic cytokines release (through the control of Granulysin and/or Granzymes (A & B) release).

Tests for determining the capacity of a compound to be a CD38 inhibitor are well known to the person skilled in the art. In a preferred embodiment, the inhibitor specifically binds to CD38 (protein or nucleic sequence (DNA or mRNA)) in a sufficient manner to inhibit the biological activity of CD38. Binding to CD38+ and inhibition of the biological activity of CD38+ may be determined by any competing assays well known in the art. For example, the assay may consist in determining the ability of the agent to be tested as a CD38 inhibitor to bind to CD38. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, an inhibitor that “specifically binds to CD38” is intended to refer to an inhibitor that binds to human CD38+ polypeptide with a KD of 1 μM or less, 100 nM or less, 10 nM or less, or 3 nM or less. Then a competitive assay may be settled to determine the ability of the agent to inhibit biological activity of CD38. The functional assays may be envisaged such as evaluating the ability to: a) inhibit processes associated with pro-cytotoxic mediator release and/or b) depleting CD8+CD38+ T cells.

The skilled in the art can easily determine whether a CD38 inhibitor neutralizes, blocks, inhibits, abrogates, reduces or interferes with a biological activity of CD38. To check whether the CD38 inhibitor binds to CD38 and/or is able to inhibit CD38 activity (or expression) such as processes associated with inhibit processes associated with pro-cytotoxic mediator release and/or depleting CD8+CD38+ T cells may be performed with each inhibitor. For instance, inhibiting pro-cytotoxic mediator release can be assessed by detecting mediators with specific antibody, ultrasensitive immunodetection (digital ELISA) as described in the Example section (see FIGS. 2 and 7), and depleting CD8+CD38+ T cells assay can be measured by the aforementioned methods such as microtitration plate coated with the binding partner for the surface marker, activated beads (ie magnetically responsive beads), flow cytometry fluorescence activated cell sorting (FACS), Cell sorting protocols using fluorescent labeled antibodies directed against the surface marker (or immunobeads coated with antibody).

In a particular embodiment, a CD38 inhibitor according to the invention can be a molecule selected from a peptide, a peptide mimetic, a small organic molecule, an antibody, an aptamer, a polynucleotide (inhibitor of CD38+ gene expression) and a compound comprising such a molecule or a combination thereof.

More particularly, the CD38 inhibitor according to the invention is:
1) an inhibitor of CD38 activity (such as, antibody, Car-T cells, aptamer) and/or
2) an inhibitor of CD38 gene expression (such as antisense oligonucleotide, nuclease,).

Antibody or an Antigen-Binding Molecule

The CD38 inhibitor can be an antibody or an antigen-binding molecule. In an embodiment, the antibody specifically recognize/bind CD38+(e.g. CD38+ of SEQ ID NO: 1) or an epitope thereof involved in the pro-cytotoxic mediator release (Granulysin and/or Granzymes (A & B) release). In another preferred embodiment, the antibody is a monoclonal antibody.

The inventors have evaluated the depletion of human CD38+CD8+ T cells with an anti-CD38 mAb (Daratumumab) in a murine model of graft versus host versus disease (GVHD) resulting in the reduction of human CD8+CD38+ T cell levels in blood and the same approach is developed to demonstrate that anti-CD38 mAb injections deplete CD8+CD38+ T cells in TEN murine models (NGS mice) using T cells collected from TEN patients (see example 2).

In preferred embodiment, the CD38 inhibitors may consist in an antibody (the term including antibody fragment or portion) directed against the CD38, that induce depletion of CD8+CD38+ T cells in such a way that said antibody impairs the cytotoxic mediator release (“neutralizing antibody”).

Then, for this invention, neutralizing antibody of CD38 are selected as above described for their capacity to (i) bind to CD38 (protein) and/or ii) inhibit processes associated with pro-cytotoxic cytokines release and/or iii) depleting CD8+CD38+ T cells.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of CD38. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant CD38 may be provided by expression with recombinant cell lines or bacteria. Recombinant form of CD38 may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,762 and 5,859,205, which are hereby incorporated by reference. The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Example of monoclonal antibody used as a CD38+ inhibitor for use in the context of the present invention can be selected from the monoclonal antibodies described in the above section
Daratumumab (Darzalex) (CAS Number: 945721-28-8/DrugBank No: DB09331) developed by Genmab and Janssen which targets CD38 is used in treating multiple myeloma and described in McKeage K (2016). Drugs. 76 (2): 275-81 and Xia C, et al (2016). Drugs of Today. 52 (10): 551-560. This FDA and EMA-approved CD38 inhibitor that may be used as a monotherapy in multiple myeloma patients who already tried at least three other therapies, including a proteasome inhibitor and an immunomodulatory agent. Daratumumab binds to a different CD38 epitope amino-acid sequence than does the anti-CD38 monoclonal antibody isatuximab (Dhillon S (2020). Drugs. 80 (9): 905-912). Daratumumab binds to CD38, causing cells apoptosis via antibody-dependent cellular cytotoxicity, complement-dependent cytotoxicity or antibody-dependent cellular phagocytosis (Konen J M, et al (2019). “Cells. 9 (1): 52; Roccatello D, et al (2020). International Journal of Molecular Sciences. 21 (11): 4129). These effects are dependent upon fragment crystallizable region immune effector mechanisms and unlike isatuximab which causes apoptosis directly, daratumumab induces apoptosis indirectly (Martin T G, et al (2019). Cells. 8 (12): 1522). Antibody-dependent cellular cytotoxicity is by means of natural killer cells

The anti CD38 monoclonal antibodies initially developed by Genmab as CD38 inhibitors (and chimeric antibodies) can also be found in patent application WO2010147171, US2009148449.
Isatuximab, (CAS Number: 1461640-62-9/DrugBank: DB14811) developed by Sanofi, is anti CD38 monoclonal antibody, which targets a particular region on the CD38 protein to trigger apoptosis (programmed cell death) and an immune response. It has been granted orphan drug status as a potential multiple myeloma therapy by the FDA and the European Medicines Agency (EMA). A biologics license application requesting its approval for people with hard-to-treat (relapsed/refractory) multiple myeloma is under FDA review. The structure of isatuximab consists of two identical immunoglobulin kappa light chains and also two equal immunoglobulin gamma heavy chains. Chemically, isatuximab is similar to the structure and reactivity of daratumumab, hence both drugs show the same CD38 targeting. However, isatuximab shows a more potent inhibition of its ectozyme function. The latter gives potential for some non-cross reactivity. Isatuximab shows action of an allosteric antagonist with the inhibition of the CD38 enzymatic activity. Additionally, isatuximab shows potential where it can induce apoptosis without cross linking (Rajan A M, Kumar S (July 2016). Blood Cancer Journal. 6 (7): e451). Lastly, Isatuximab reveals direct killing activity when a larger increase in apoptosis is detected in CD38 expressing cancer cells. Furthermore, isatuximab demonstrated a dose dependent inhibition of CD38 enzymatic activity (Martin T, et al. (June 2017). Blood. 129 (25): 3294-3303).

The anti CD38 monoclonal antibodies initially developed by Sanofi as CD38 inhibitors (and chimeric antibodies) can also be found in patent application WO2008047242, WO2012041800 (which disclosed light and heavy chain variable regions sequence of isatuximab as SEQ ID NO: 22 and SEQ ID NO: 21).
MOR202 (CAS Number 2197112-39-1) is a CD38-binding antibody being developed by Morphosys. The activity of MOR202, a fully human anti-CD38 antibody, induces Myeloma Multiple (MM) cell death by ADCC, ADCP, and CDC. Similar to Daratumumab, MOR202 induces MM cell death requiring the presence of a cross-linking agent (van de Donk NWCJ, et al. Blood. 2018; 131(1):13-29). It is not clear whether MOR202 has immunomodulatory functions. Preclinical data indicate that MOR202 reduces NK cells (Casneuf T, et al. Blood Adv. 2017; 1(23):2105-2114). Three clinical trials on MM with MOR202 are ongoing to evaluate the response and side-effect as a single agent or in combination therapy. In a first-in-human, multicenter, phase I/IIa clinical trial (ClinicalTrials.gov, NCT01421186), MOR202 was proved to be safe and effective either as monotherapy or in combination with dexamethasone or dexamethasone and an immunomodulatory drug for RRMM (relapsed or refractory multiple myeloma) population when the doses were up to 16 mg/kg by intravenous infusion (Raab M S, et al. Lancet Haematol. 2020; 7(5): e381-e394).
TAK-079 is a CD38-binding antibody being developed by Takeda. It is currently being tested, in combination with standard-of-care therapy, in a Phase 1 clinical trial (NCT03984097) in newly diagnosed patients, and in Phase 1/2 trial (NCT03439280) in those with advanced multiple myeloma. TAK-079, a fully human IgG 1 λ mAb, binds to and kills both human and monkey CD38+ cells, which majorly depends on CDC, ADCC, and ADCP (Korver W, et al. Pharmacol Rev. 2019; 370(2):182-196). Similar to MOR202, it is not clear whether TAK-079 has immunomodulatory function. TAK-079 has been tested in healthy populations and found to be well tolerated (Fedyk E R, et al. Br J Clin Pharmacol. 2020; 86(7):1314-1325). TAK-079 by subcutaneous injection was more durable in depleting plasmablasts and NK cells, which would facilitate curing malignant CD38+ plasma or NK cell disease (Fedyk E R, et al. Br J Clin Pharmacol. 2020; 86(7):1314-1325). In a Phase Ib clinical trial, safe and well-tolerated TAK-079 resulted in a 43% objective response rate for heavily pre-treated patients with RRMM by subcutaneous injection (https://doi-org.proxy.ins ermbiblio.inist.fr/10.1182/blood-2019-128007).
AMG424, being developed by Amgen, and GBR 1342, being developed by Glenmark Pharmaceuticals are both bispecific antibodies against CD3 and CD38 (CD38xCD3 BsAbs). CD3 is a protein found on the surface of T-cells and by binding to CD3, the bispecific antibodies is thought to activate T-cells, directing them against CD38-producing cells. They are both (AMG424 as GBR1342) based on the structure of Fab-Fc (G1) x scFv-Fc (G1) with a hetero-Fc domain lack of Fcγ receptor and complement binding (Labrijn A F, et al Nat Rev Drug Discov. 2019; 18(8):585-608). The both CD38xCD3 BsAbs eliminate CD38+ cancer cells via simultaneously binding to CD38 expressed on cancer cells and CD3 expressed on T cells, triggering T-cell activation, proliferation, and release of cytokine (Drent E, et al. Haematologica. 2016; 101(5):616-625). The ineffective Fc domain determines the deficiency of classic Fc-dependent immune effector mechanisms. Antigen-independent cytokine release syndrome (CRS) might occur on condition that Fc regions of BsAbs bind Fcγ receptors on T cells, which may cause nonspecific activation of T cells (Chatenoud L, et al. Transplantation. 1990; 49(4):697-702). To prevent the off-target toxicity, researchers introduced the mutational Fc domain to bsTCEs, which could improve T-cell trafficking and antitumor potency (Wang L, et al. Cancer Immunol Res. 2019; 7(12):2013-2024 and Woodle E S, et al. Transplantation. 1999; 68(5):608-616). Clinical trials in phase I with AMG424 (ClinicalTrials.gov, NCT03445663) and GBR1342 (ClinicalTrials.gov, NCT03309111) are ongoing, which aim at RRMM and MM, respectively. GBR 1342 has been named an orphan drug as a potential treatment for previously-treated multiple myeloma patients.

As the CD38 is a transmembrane target express on specific subtype of cytotoxic CD8 T cells, the antibody of the invention acting as an activity inhibitor could be an antibody drug conjugates (or ADC).

In some embodiments, the antibody of the present invention is conjugated to a therapeutic moiety, i.e. a drug. The therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Scapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

In some embodiments, the antibody is conjugated, e.g., as a fusion protein, to a lytic peptide such as CLIP, Magainin 2, mellitin, Cecropin and P18.

In some embodiments, the antibody is conjugated to a cytokine, such as, e.g., IL-2, IL-4, IL-6, IL-7, IL-10, IL-12, IL-13, IL-15, IL-18, IL-23, IL-24, IL-27, IL-28a, IL-28b, IL-29, KGF, IFNa, IFN3, IFNy, GM-CSF, CD40L, Flt3 ligand, stem cell factor, ancestim, and TNFa.

In some embodiments, the antibody is conjugated to a radioisotope or to a radioisotope-containing chelate. For example, the antibody can be conjugated to a chelator linker, e.g. DOTA, DTPA or tiuxetan, which allows for the antibody to be complexed with a radioisotope. The antibody may also or alternatively comprise or be conjugated to one or more radiolabeled amino acids or other radiolabeled molecules Non-limiting examples of radioisotopes include 3H, 14C, 15N, 35S, 90Y, 99Tc, 1251, 1311, 186Re, 213Bi, 225Ac and 227Th. For therapeutic purposes, a radioisotope emitting beta- or alpha-particle radiation can be used, e.g., 1311, 90Y, 211At, 212Bi, 67Cu, 186Re, 188Re, and 212Pb.

In certain embodiments, an antibody-drug conjugate comprises an anti-tubulin agent. Examples of anti-tubulin agents include, for example, taxanes (e.g., Taxol® (paclitaxel), Taxotere® (docetaxel)), T67 (Tularik), vinca alkyloids (e.g., vincristine, vinblastine, vindesine, and vinorelbine), and dolastatins (e.g., auristatin E, AFP, MMAF, MMAE, AEB, AEVB). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin. In some embodiments, the cytotoxic agent is a maytansinoid, another group of anti-tubulin agents. For example, in specific embodiments, the maytansinoid is maytansine or DM-1 (ImmunoGen, Inc.; see also Chari et al., Cancer Res. 52:127-131, 1992).

In other embodiments, the cytotoxic agent is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, or trifluridine.

In other embodiments, an anti-CD38 antibody is conjugated to a pro-drug converting enzyme. The pro-drug converting enzyme can be recombinantly fused to the antibody or chemically conjugated thereto using known methods. Exemplary pro-drug converting enzymes are carboxypeptidase G2, β-glucuronidase, penicillin-V-amidase, penicillin-G-amidase, β-lactamase, β-glucosidase, nitroreductase and carboxypeptidase A.

Example of anti-CD38 antibody drug conjugated used as a CD38 inhibitor for use in the context of the present invention can be TAK-169 developed by Takeda is a toxic agent, which is designed to internalize and kill CD38-positive cells by blocking protein synthesis. TAK-169 comprising a de-immunized form of the ribosome inactivating Shiga-like toxin A-subunit (SLTA) genetically fused to an antibody fragment that specifically targets the CD38 cell surface receptor. It is in a safety and early efficacy Phase 1 clinical trial (NCT04017130) in people with relapsed/refractory multiple myeloma.

Aptamer

The CD38 inhibitor can also be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Polynucleotide

The CD38 inhibitor can also be a polynucleotide, typically an inhibitory nucleotide. (Inhibitor of CD38 gene expression). In one embodiment, the inhibitor of CD38 gene expression antibody specifically recognize/bind CD38 nucleic acid sequence (e.g. CD38 of SEQ ID NO: 2)

These polynucleotides include short interfering RNA (siRNA), microRNA (miRNA), and synthetic hairpin RNA (shRNA), anti-sense nucleic acids, complementary DNA (cDNA) or guide RNA (gRNA usable in the context of a CRISPR/Cas system). In some embodiments, a siRNA targeting CD38+ expression is used. Interference with the function and expression of endogenous genes by double-stranded RNA such as siRNA has been shown in various organisms. See, e.g., Zhao Y et al, “Co-delivery of CD38+ siRNA and statin to endothelial cells and macrophages in the atherosclerotic lesions by a dual-targeting core-shell nanoplatform: A dual cell therapy to regress plaques,” Journal of Controlled Release Volume 283, 10 Aug. 2018, p.241-260; Arjuman A et al “CD38: A potential target for therapy in atherosclerosis; an in vitro study “Int J Biochem Cell Biol. 2017 October; 91(Pt A): 65-80. doi: 10.1016. siRNAs can include hairpin loops comprising self-complementary sequences or double stranded sequences. siRNAs typically have fewer than 100 base pairs and can be, e.g., about 30 bps or shorter, and can be made by approaches known in the art, including the use of complementary DNA strands or synthetic approaches. Such double-stranded RNA can be synthesized by in vitro transcription of single-stranded RNA read from both directions of a template and in vitro annealing of sense and antisense RNA strands. Double-stranded RNA targeting CD38 can also be synthesized from a cDNA vector construct in which a CD38 gene (e.g., human CD38 gene) is cloned in opposing orientations separated by an inverted repeat. Following cell transfection, the RNA is transcribed and the complementary strands reanneal. Double-stranded RNA targeting the CD38+ gene can be introduced into a cell (e.g., a tumor cell) by transfection of an appropriate construct.

Typically, RNA interference mediated by siRNA, miRNA, or shRNA is mediated at the level of translation; in other words, these interfering RNA molecules prevent translation of the corresponding mRNA molecules and lead to their degradation. It is also possible that RNA interference may also operate at the level of transcription, blocking transcription of the regions of the genome corresponding to these interfering RNA molecules.

The structure and function of these interfering RNA molecules are well known in the art and are described, for example, in R. F. Gesteland et al., eds, “The RNA World” (3rd, Cold Spring Harbor Laboratory Press, Cold Spring Harbor, N.Y., 2006), pp. 535-565, incorporated herein by this reference. For these approaches, cloning into vectors and transfection methods are also well known in the art and are described, for example, in J. Sambrook & D. R. Russell, “Molecular Cloning: A Laboratory Manual” (3rd, Cold Spring Harbor Laboratory Press, Cold Spring Harbor, 2001), incorporated herein by this reference.

In addition to double stranded RNAs, other nucleic acid agents targeting CD38+ can also be employed in the practice of the present invention, e.g., antisense nucleic acids. Antisense nucleic acids are DNA or RNA molecules that are complementary to at least a portion of a specific target mRNA molecule. In the cell, the single stranded antisense molecule hybridizes to that mRNA, forming a double stranded molecule. The cell does not translate an mRNA in this double-stranded form. Therefore, antisense nucleic acids interfere with the translation of mRNA into protein, and, thus, with the expression of a gene that is transcribed into that mRNA. Antisense methods have been used to inhibit the expression of many genes in vitro. See, e.g., Li D et al., “Antisense to CD38+ inhibits oxidized LDL-mediated upregulation of monocyte chemoattractant protein-1 and monocyte adhesion to human coronary artery endothelial cells “Circulation. 2000 Jun. 27; 101 (25):2889-95. doi: 10.1161; Amati F et al, “CD38+ Inhibition in ApoE KO Mice Using a Schizophyllan-based Antisense Oligonucleotide Therapy,” Mol Ther Nucleic Acids. 2012 December; 1(12): e58; incorporated herein by this reference. CD38+ polynucleotide sequences from human and many other animals in particular mammals have all been delineated in the art. Based on the known sequences, inhibitory nucleotides (e.g., siRNA, miRNA, or shRNA) targeting CD38+ can be readily synthesized using methods well known in the art.

Exemplary siRNAs according to the invention could have up to 29 bps, 25 bps, 22 bps, 21 bps, 20 bps, 15 bps, 10 bps, 5 bps or any integral number of base pairs between these numbers. Tools for designing optimal inhibitory siRNAs include that available from DNAengine Inc. (Seattle, Wash.) and Ambion, Inc. (Austin, Tex).

Example of commercial siRNAs against CD38+ are available.

The guide RNA (gRNA) sequences direct a nuclease (i.e. CrispRCas9 protein) to induce a site-specific double strand break (DSB) in the genomic DNA in the target sequence.

Accordingly, Inhibitors of CD38 gene expression for use in the present invention may be based nuclease therapy (like Talen or Crispr).

The term “nuclease” or “endonuclease” means synthetic nucleases consisting of a DNA binding site, a linker, and a cleavage module derived from a restriction endonuclease which are used for gene targeting efforts. The synthetic nucleases according to the invention exhibit increased preference and specificity to bipartite or tripartite DNA target sites comprising DNA binding (i.e. TALEN or CRISPR recognition site(s)) and restriction endonuclease target site while cleaving at off-target sites comprising only the restriction endonuclease target site is prevented.

The guide RNA (gRNA) sequences direct the nuclease (i.e. Cas9 protein) to induce a site-specific double strand break (DSB) in the genomic DNA in the target sequence.

Restriction endonucleases (also called restriction enzymes) as referred to herein in accordance with the present invention are capable of recognizing and cleaving a DNA molecule at a specific DNA cleavage site between predefined nucleotides. In contrast, some endonucleases such as for example Fok1 comprise a cleavage domain that cleaves the DNA un-specifically at a certain position regardless of the nucleotides present at this position. Therefore, preferably the specific DNA cleavage site and the DNA recognition site of the restriction endonuclease are identical. Moreover, also preferably the cleavage domain of the chimeric nuclease is derived from a restriction endonuclease with reduced DNA binding and/or reduced catalytic activity when compared to the wildtype restriction endonuclease.

According to the knowledge that restriction endonucleases, particularly type II restriction endonucleases, bind as a homodimer to DNA regularly, the chimeric nucleases as referred to herein may be related to homodimerization of two restriction endonuclease subunits. Preferably, in accordance with the present invention the cleavage modules referred to herein have a reduced capability of forming homodimers in the absence of the DNA recognition site, thereby preventing unspecific DNA binding. Therefore, a functional homodimer is only formed upon recruitment of chimeric nucleases monomers to the specific DNA recognition sites. Preferably, the restriction endonuclease from which the cleavage module of the chimeric nuclease is derived is a type 11P restriction endonuclease. The preferably palindromic DNA recognition sites of these restriction endonucleases consist of at least four or up to eight contiguous nucleotides. Preferably, the type 11P restriction endonucleases cleave the DNA within the recognition site which occurs rather frequently in the genome, or immediately adjacent thereto, and have no or a reduced star activity. The type 11P restriction endonucleases as referred to herein are preferably selected from the group consisting of: Pvu11, EcoRV, BamH1, Bcn1, BfaSORF1835P, BfiI, Bg11, Bg111, BpuJ1, Bse6341, BsoB1, BspD6I, BstY1, Cfr101, Ec118k1, Eco01091, EcoR1, EcoR11, EcoRV, EcoR1241, EcoR12411, HinP11, Hinc11, Hind111, Hpy991, Hpy1881, Msp1, Mun1, Mva1, Nae1, NgoMIV, Not1, OkrAa, Pab1, Pac1, PspG1, Sau3A1, Sda1, Sfi1, SgrA1, Tha1, VvuYORF266P, Dde1, Eco571, Hae111, Hha11, Hind11, and Nde1.

Example of commercial gRNAs against CD38 are available.

Other nuclease for use in the present invention are disclosed in WO 2010/079430, WO2011072246, WO2013045480, Mussolino C, et al (Curr Opin Biotechnol. 2012 October; 23(5):644-50) and Papaioannou I. et al (Expert Opinion on Biological Therapy, March 2012, Vol. 12, No. 3: 329-342) all of which are herein incorporated by reference.

Ribozymes can also function as inhibitors of CD38 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of CD38 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of CD38 gene expression can be prepared by known methods. These include techniques for chemical synthesis such as, e.g., by solid phase phosphoramadite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing CD38. Preferably, the vector transports the nucleic acid within cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vectors and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adenoviruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, nuclease (i.e. CrispR), siRNA, shRNA or ribozyme nucleic acid sequences are under the control of a heterologous regulatory region, e.g., a heterologous promoter. The promoter may be specific for the T cells.

CarT Cells

The CD38 inhibitor can also be a T cell characterized in that it expresses a chimeric antigen receptor which recognizes/binds CD38.

Typically, said chimeric antigen receptor comprises at least one VH and/or VL sequence of the antibody of the present invention. The chimeric antigen receptor used in the context of the present invention also comprises an extracellular hinge domain, a transmembrane domain, and an intracellular T cell signaling domain.

As used herein, the term “chimeric antigen receptor” or “CAR” has its general meaning in the art and refers to an artificially constructed hybrid protein or polypeptide containing the antigen binding domains of an antibody (e.g., scFv) linked to T- cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

In some embodiments, the CAR comprises an extracellular hinge domain, a transmembrane domain, and an intracellular T cell signaling domain selected from the group consisting of CD28, 4-1BB, and CD3ζ intracellular domains. CD28 is a T cell marker important in T cell co-stimulation. 4-1BB transmits a potent costimulatory signal to T cells, promoting differentiation and enhancing long-term survival of T lymphocytes. CD3ζ associates with TCRs to produce a signal and contains immunoreceptor tyrosine-based activation motifs (ITAMs).

In some embodiments, the chimeric antigen receptor used in the context of the present invention can be glycosylated, amidated, carboxylated, phosphorylated, esterified, N-acylated, cyclized via, e.g., a disulfide bridge, or converted into an acid addition salt and/or optionally dimerized or polymerized.

A host cell comprising a nucleic acid encoding for a chimeric antigen receptor is used to generate CAR T cells. While the host cell can be of any cell type, can originate from any type of tissue, and can be of any developmental stage; the host cell is a T cell, e.g. isolated from peripheral blood lymphocytes (PBL) or peripheral blood mononuclear cells (PBMC). In some embodiments, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell can be any type of T cell and can be of any developmental stage, including but not limited to, CD4+/CD8+ double positive T cells, CD4+ helper T cells, e.g., Th2 cells, CD8+ T cells (e.g., cytotoxic T cells), tumor infiltrating cells, memory T cells, naive T cells, and the like. The T cell may be a CD8+ T cell or a CD4+ T cell.

The population of those T cells prepared as described above can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. Adoptive immunotherapy of cancer refers to a therapeutic approach in which immune cells with an antitumor reactivity are administered to a tumor-bearing host, with the aim that the cells mediate either directly or indirectly, the regression of an established tumor. Transfusion of lymphocytes, particularly T lymphocytes, falls into this category. Currently, most adoptive immunotherapies are autolymphocyte therapies (ALT) directed to treatments using the patient's own immune cells. These therapies involve processing the patient's own lymphocytes to either enhance the immune cell mediated response or to recognize specific antigens or foreign substances in the body, including the cancer cells. The treatments are accomplished by removing the patient's lymphocytes and exposing these cells in vitro to biologics and drugs to activate the immune function of the cells. Once the autologous cells are activated, these ex vivo activated cells are reinfused into the patient to enhance the immune system to treat cancer. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the T cells with the desired specificity, on the age and weight of the recipient, on the severity of the targeted condition and on the immunogenicity of the targeted Ags. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. For example, if cells that are specific for a particular Ag are desired, then the population will contain greater than 70%, generally greater than 80%, 85% and 90-95% of such cells. For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

Example of Car T cells used as a CD38 inhibitor for use in the context of the present invention can be the chimeric antigen receptor T-cells (CAR-T cells) against CD38 developed by Sorrento Therapeutics. CAR-T CD38 cells are designed to bind to and selectively kill cells that have high levels of CD38 on their surface, such as myeloma cells. The therapy is currently in Phase 1 clinical trial (NCT03464916) in advanced multiple myeloma patients. Preclinical data of CD38-CAR-T cells showed a significant effect on eliminating MM cells in vitro and in vivo and primary malignant cells isolated from patients with MM in vitro, though original CD38 expression was disappeared after the treatment with CD38-CAR-T cells (DrentE, et al. Haematologica. 2016; 101(5):616-625).

Therapeutic Method of a Specific Population

The invention also relates to a method for treating Toxic Epidermal Necrolysis with a CD38+ inhibitor in a subject having a high level of CD8+CD45RA−CCR7−CD38+ T lymphocytes in a biological sample, wherein the level of said population of T lymphocytes obtained from said subject, have been detected by one of method of the invention.

In a preferred embodiment, the biological sample is blood sample or immune primary cells or skin sample.

In the context of the invention, the term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies.

In a particular embodiment, a CD38+ inhibitor according to the invention can be a molecule selected from a peptide, a peptide mimetic, a small organic molecule, an antibody, an aptamer, a phospholipid, a polynucleotide (inhibitor of CD38+ gene expression) and a compound comprising such a molecule or a combination thereof.

Another object of the present invention is a method of treating Toxic Epidermal Necrolysis in a subject comprising the steps of:
a) providing a sample containing T lymphocytes from a subject,
b) detecting the level of a population of CD8+CD45RA−CCR7−CD38+ T lymphocytes,
c) comparing the level determined at stet b) with a reference value and
if level determined at stet b) is higher than the reference value, treating the subject with an CD38 inhibitor.

In a specific embodiment when the sample is a skin blister, a skin biopsy or a blood sample, the level of expression of the TEN-associated T lymphocyte biomarker (“Biomarker”: CD8+CD45RA−CCR7−/CD38+ T cells) is detected by clonal expansion

The invention will be further illustrated by the following figures and examples. However, these examples and figures should not be interpreted in any way as limiting the scope of the present invention.

### FIGURES

FIG. 1: Immunophenotyping of leucocytes present in skin samples from TEN, MPE or healthy donors. The leucocytes isolated from the blisters of 7 TEN patients (A), and the skin of 6 MPE patients (B) and 4 healthy donors (C) were analyzed by mass cytometry. Scatter plots depict percentages of conventional TCRαβ+ lymphocytes, gamma delta T cells, B lymphocytes, NK cells, monocytes or conventional dendritic cells in CD45+ hematopoietic cells (A1-C1), and percentages of CD8+, CD4+, double negative and double positive T cell subsets, as well as iNKT and MAIT cells in gated TCRαβ+ population (A2-C2). Mean frequencies +/−SD are also shown. Statistics compared frequencies of each subset in TEN versus MPE (*) or healthy (^{£}) donor samples. ^{*,£}P<0.05, Mann-Whitney test.

FIG. 2: High-dimensional cell analysis of CD8+ T cells identifies TEN-enriched immunophenotypes. FlowSOM analysis with automatic consensus clustering was performed on concatenated CD8+ T cell data (300 cells/sample) from both lesion (blisters/skin) and PBMC samples from TEN and MPE patients and healthy donors. (A) Heat map of the integrated MFI of 16 markers across the 7 FlowSOM clusters (data not shown). The figures in the heatmap represent the median of the arcsinh for each cluster (centroid) with 0-1 transformed marker expression. Clusters (columns) and markers (rows) were hierarchically metaclustered using Ward's method to group subpopulations with similar phenotype. (B) Cluster frequencies were determined for each sample from each subject, to understand tissue abundance. Statistics compared frequencies of each cluster in PBMC or skin samples versus the frequency of the respective cluster in healthy donor samples. ns=not significant, *P<0.05, ***P<0.01, Mann-Whitney test (two-tailed).

FIG. 3: TCR Vβ repertoire usage in T cell subsets isolated from the lesional skin of TEN and MPE patients. The leucocytes isolated from the blisters of 13 subjects with TEN (A & C) and the lesional skin of 5 subjects with MPE (B & D) were analysed by flow cytometry. Histograms depict percentages of the 24 TCR Vβ chains in gated CD8+ (A & B) and CD4+ (C & D) T cell subsets, using the IOTest® Beta Mark TCR Vβ Repertoire Kit (TCR-Vβ1, 2, 3, 4, 5.2, 5.3, 7.1, 7.2, 8, 9, 11, 12, 13.1, 13.2, 13.6, 14, 16, 17, 18, 20, 21.3, 22, 23). Each symbol (triangles for TEN, squares for MPE) represents a different subject.

The black bar illustrates the threshold value from which TCR Vβ chains were considered as highly expanded (using Tukey's rule for the detection of outliers, i.e. Q3+1.5×IQR).

FIG. 4: Increased clonality indices in TEN blister but not TEN PBMC samples. TCR repertoire diversity was evaluated by high-throughput sequencing (HTS) on total blister and skin (A) and PBMC (B) samples from 15 subjects with TEN and 7 subjects with MPE. Scatter-plots depict Shannon entropy-based clonality indices for total productive TCR rearrangements. Exact dates of sample collection are reported in Table S1. Values approaching 1 indicate a highly clonal repertoire in which a small number of rearrangements comprise a large portion of all immune cells. Conversely values approaching 0 indicate a polyclonal repertoire where all rearrangements are present at an identical frequency. **P<0.01. Student t test (two-tailed).

FIG. 5: The percentage of maximal skin detachment in TEN patients correlates with clonality indices and clonal expansion of skin clones in PBMCs. Quantification of skin detachment (expressed as percentage of total body area) in 15 TEN patients was appreciated at their arrival at hospital (initial) and at the peak of the skin reaction (maximal) (A). The latter was compared with the Shannon entropy-based clonality indices determined in blisters (B) and PBMCs (C). Correlations between clinical severity and the percentages of the top clone (C) or the cumulative percentages of the highly expanded clones (HEC) are also provided (D). Respective correlation factors were calculated using Pearson correlation method. The coefficient of determination, R², and statistical significance are indicated for each correlation. *P<0.05. Student t test (two-tailed)

FIG. 6: Immunophenotype of dominant TCRVβ+ cells. The dominant CD8+ TCRVβ+ cell subset isolated from the blister fluids of 4 subjects with TEN (TEN-3, 7, -9 and -10) was analyzed for the expression of CD38 and Granulysin, by mass cytometry (A). Pictures depict representative gating strategy to select the dominant CD8+ TCRVβ+ cell subset (TCR-Vβ21.3+ for TEN-3, TCR-Vβ13.2+ for TEN-9 or TEN-7 and see* for TEN-10; A1) and histogram overlays of CD38 and Granulysin expression, when compared with non-dominant CD8+ TCRVβ (others) or CD4+ T cell counterparts (A2). To characterize the phenotypic identity of respective subsets, CyTOF data were superimposed on concatenated CD8+ T cell clusters identified in FIG. 2. Histograms depict the frequency of each cluster in dominant and non-dominant CD8+ TCRVβ+ cell subsets (B).

*Of note, as no anti-Vβ3 mAb exists for CyTOF, dominant TCRVβ3+ cells in patient TEN-10 (which represent 90% of total CD8+ T cells in skin) were gated by negative selection. We gated cells negative for TCR-Vβ21.3+, -Vβ13.2+ and -Vβ7.2+ expression.

FIG. 7: Depletion of CD8+CD38+ T lymphocytes with an anti-CD38+ monoclonal antibody

NGS mice were reconstituted with 10×10⁶ PBMCs from a healthy donor, and treated by two-weekly injections of an anti-CD38+ mAb (Daratumumab, at 100 or 300 microg/mouse). Control group received PBS. Results depict the percentage +/−SD of CD38+ fraction among human CD8+ T cells present in the spleen, as evaluated by flow cytometry 28 days after PBMCs injection.

FIG. 8: Percentage of humanization 7 days after mAb injection

NGS mice were reconstituted with 10×10⁶ PBMCs from a healthy donor, and treated by two-weekly injections of an anti-CD38+ mAb (Daratumumab, at 100 or 300 microg/mouse). Control group received PBS. The percentage of humanization was measured by flow cytometry at day 5 and at day 12 after PBMC injection. It was calculated by dividing the percentage of human blood CD45+ cells/the percentage of total (mouse+human) blood CD45+ cells.

FIG. 9: Kinetics of human PBMC expansion in lamotrigine- and vehicle-treated NSG recipient mice.

NSG animals were adoptively transferred at day 0 with 1.10⁶ millions PBMCs collected from a TEN patient, 1 year after disease recovery. Animals were then administrated with lamotrigine (the culprit drug; 0.1 mg/kg/day) or vehicle by oral gavage, every day, from day 4. Results depict the kinetic of human CD45+ cell expansion measured by flow cytometry in the blood of NSG mice throughout the protocol, or the spleen at day 29. Results are expressed as mean and individual % of humanization, calculated according to the following formula: % human CD45+ cells/% (mouse+human) CD45+ cells. Six mice per group were used in this experiment.

FIG. 10: Preferential expansion of TEN PBMCs in lamotrigine-treated NSG recipient mice.

NSG animals were adoptively transferred at day 0 with 1.10⁶ millions PBMCs collected from a TEN patient (1 year after disease recovery) or from a healthy donor. Animals were then administrated with lamotrigine (the culprit drug; 0.1 mg/kg/day) or vehicle, by oral gavage every day, from day 4. Results depict the expansion of human CD45+ cells measured by flow cytometry in the spleen of NSG mice, 29 days after cell transfer. Results are expressed as mean and individual % of humanization, calculated according to the following formula: % human CD45+ cells/% (mouse+human) CD45+ cells. Six mice per group were used in this experiment.

FIG. 11: A significant part of expanded CD8+ T cells in lamotrigine-treated NSG recipient mice expressed CD38 and Granzyme B and Granulysin markers.

NSG animals were adoptively transferred at day 0 with 1.10⁶ millions PBMCs collected from a TEN patient (1 year after disease recovery). Animals were then administrated with lamotrigine (the culprit drug; 0.1 mg/kg/day), by oral gavage every day, from day 4. Results depict the expression of CD38 (A) and Granzyme B and Granulysin (B) markers on CD4+ and CD8+ T cells as measured by flow cytometry in the spleen of NSG mice, 43 days after cell transfer.

FIG. 12: Daratumamub injections in preventive mode preferentially depleted CD8+Vbeta7.1+ T cells in NSG recipient mice.

NSG animals were adoptively transferred at day 0 with 1.10⁶ millions PBMCs collected from a TEN patient, and successively administrated with lamotrigine (the culprit drug; every day, from day 4. In parallel, NSG mice were injected i.p. with 200 mg of daratumumab or a control isotype twice weekly, starting mAb injections from day 4 (preventive mode). Results depict the kinetic of human CD8+Vbeta7.1+ T cell expansion measured by flow cytometry in the spleen of NSG mice at day 31. Results are expressed as mean and individual % of Vbeta7.1+ cell fraction among total CD8+ T cells. Ten mice per group were used in this experiment. Of note, 3.2% of Vbeta7.1+ cells were detected in CD8+ T cells at the time of cell transfer (day 0).

FIG. 13: Daratumamub injections in preventive mode blocked the expansion of both CD4+CD38+ and CD8+CD38+ T cells in NSG recipient mice.

NSG animals were adoptively transferred at day 0 with 1.10⁶ millions PBMCs collected from a TEN patient, and successively administrated with lamotrigine (the culprit drug; every day, from day 4. In parallel, NSG mice were injected i.p. with 200 mg of daratumumab or a control isotype twice weekly, starting mAb injections from day 4 (preventive mode). Results depict the kinetic of human CD4+CD38+/− and CD8+CD38+/− T cell expansion measured by flow cytometry in the blood of NSG mice throughout the protocol. Results are expressed as mean and individual number of CD38+(A) and CD38− (B) cells/mL blood. Ten mice per group were used in this experiment.

FIG. 14: Lower expansion of both CD4+ and CD8+ T cells in daratumumab-treated NSG recipient mice.

NSG animals were adoptively transferred at day 0 with 1.10⁶ millions PBMCs collected from a TEN patient, and successively administrated with lamotrigine (the culprit drug; or vehicle by oral gavage, every day, from day 4. In parallel, NSG mice were injected i.p. with 200 mg of daratumumab or a control isotype twice weekly, starting mAb injections from day 4 (preventive mode). Results depict the kinetic of human CD4+ and CD8+ T cell expansion measured by flow cytometry in the blood of NSG mice throughout the protocol. Results are expressed as mean and individual number of CD4+ and CD8+ T cells/mL blood. Ten mice per group were used in this experiment.

FIG. 15: Daratumamub injections in preventive mode strongly inhibited the expansion of cytotoxic CD8+CD38+ T cells in NSG recipient mice.

NSG animals were adoptively transferred at day 0 with 1.10⁶ millions PBMCs collected from a TEN patient, and successively administrated with lamotrigine (the culprit drug; 0.1 mg/kg/day), every day, from day 4. In parallel, NSG mice were injected i.p. with 200 mg of daratumumab or a control isotype twice weekly, starting mAb injections from day 4 (preventive mode). Results depict the kinetic of human cytotoxic CD8+CD38+/−GranzymeB+Granulysin+ T cell expansion measured by flow cytometry in the blood of NSG mice throughout the protocol. Results are expressed as mean and individual number of CD8+CD38+GranzymeB+Granulysin+ and CD8+CD38+GranzymeB+Granulysin+ cells/mL blood (A). Details for total CD8+GranzymeB+Granulysin+ T cells are also shown (B). Ten mice per group were used in this experiment.

FIG. 16: Daratumamub injections in curative mode efficiently depleted CD8+CD38+ T cells in NSG recipient mice.

NSG animals were adoptively transferred at day 0 with 1.10⁶ millions PBMCs collected from a TEN patient, and successively administrated with lamotrigine (the culprit drug; every day, from day 4. In parallel, NSG mice were injected i.p. with 200 mg of daratumumab or a control isotype twice weekly, starting mAb treatment from day 4 (preventive mode) or from day 29 (curative mode). Results depict the kinetic of human CD8+CD38+/− T cell expansion measured by flow cytometry in the blood of NSG mice throughout the protocol. Results are expressed as mean and individual % of CD38+ and CD38− fractions among total CD8+ T cells. Five mice per group were used in this experiment.

FIG. 17: Daratumamub injections in curative mode efficiently depleted cytotoxic CD8+CD38+ T cells in NSG recipient mice.

NSG animals were adoptively transferred at day 0 with 1.10⁶ millions PBMCs collected from a TEN patient, and successively administrated with lamotrigine (the culprit drug; every day, from day 4. In parallel, NSG mice were injected i.p. with 200 mg of daratumumab or a control isotype twice weekly, starting mAb treatment from day 4 (preventive mode) or from day 29 (curative mode). Results depict the kinetic of human cytotoxic CD8+ T cell expansion measured by flow cytometry in the blood of NSG mice throughout the protocol. Results are expressed as mean and individual number of CD8+GranzymeB+Granulysin+CD38+/− T cells/mL blood. Five mice per group were used in this experiment.

### EXAMPLE

Methods:

Study Design

Patients were prospectively recruited by the drug allergy reference center at the Hospices Civils de Lyon (France) between 2014 and 2018. TEN or MPE diagnoses were based on the definition published by the RegiSCAR study group (43) (44). Only patients with a probable or a definite diagnosis of TEN or MPE were enrolled in this study. Culprit drugs in TEN patients were determined according to the Algorithm for Drug Causality for Epidermal Necrolysis (ALDEN) (45). For MPE patients, the main putative drug was also determined. We collected demographic and clinical information, including sex and age, as well as underlying diseases (i.e. the disease the culprit drug was prescribed for), comorbidities, duration of drug exposure before TEN/MPE onset and HLA-A/B genotyping results. HLA-A/B genotypes were determined by reverse PCR-sequence-specific oligonucleotide hybridization (LABType® SSO, One Lambda). Complementary information were also collected for TEN patients: SCORTEN (SCORe of Toxic Epidermal Necrosis) at diagnosis, which aim to predict the severity of the disease (46) and percentage of skin detachment assessed by E-Burn® smartphone application (Android Play store®). The latter was determined when the patient was first diagnosed with TEN (‘initial’), and when maximum involvement was observed (‘final’). We enrolled 20 healthy donors as controls.

Local ethical committee approved the study and written informed consent was obtained from each participant. Given the observational nature of the translational study, there was no randomization or formal blinding process for the investigators.

Sample Collection and Processing

Skin Samples

Skin samples for TEN mainly consisted of blister fluids and for 3 patients blister fluids and skin biopsies. Supernatant was collected and cells were repeatedly washed in complete RPMI before subsequent processing. In cases of MPE and patients TEN-15, -17 and -18, 6-mm² biopsies were performed directly into lesional erythematous skin. Abdominal skin leftovers, from healthy donors undergoing elective plastic surgery, were used as control biopsies. Skin cells were extracted by mechanical dissociation and enzymatic digestion (2 hours at 37° C. in RPMI supplemented with collagenase type 1 (1.25 U/mL, Sigma-Aldrich, Saint Quentin Fallavier, France), DNAse (4 KU/mL, Sigma-Aldrich) and HEPES buffer (5%)), before to be passed through a 100 mm cell strainer (ThermoFischer Scientific, Dardilly, France) to obtain single cell suspensions. Cell viability was determined by trypan blue exclusion.

Blood Samples

PBMCs from healthy donors and patients were isolated from whole blood samples (in Lithium-Heparin coated tubes) using Ficoll-histopaque (Ficoll-Paque PLUS®, GE Healthcare Life Sciences®) density gradient centrifugation, and cell viability was assessed as described above.

Depending on experiments, samples were either frozen in liquid nitrogen according to standard procedures, or immediately stained for immunophenotyping analysis.

Flow Cytometry Analysis

Flow cytometry was carried out using fluorescently labelled mAbs, recognizing human CD3 (7D6; Thermo Fisher Scientific, Les Ulis, France), CD4 (VIT4; Miltenyi biotech, Bergish Glabach, Germany) and CD8 (SK1; Biolegend, San Diego, California, USA) proteins. V-beta (Vβ) chain repertoire expression was assessed using a kit of 24 TCR-Vβ mAbs (IOTest® BetaMark, Beckman Coulter, Roissy, France; which includes approx. 70% of the expressed human TCR Vβ domains: TCR Vβ 1, 2, 3, 4, 5.1, 5.2, 5.3, 7.1, 7.2, 8, 9, 11, 12, 13.1, 13.2, 13.6, 14, 16, 17, 18, 20, 21.3, 22, 23) and viability discrimination was performed by incubating cells with Live/dead eFluor-506 (eBioscience, San Diego, California, USA).

Cells were analyzed on a LSR FORTESSA flow cytometer (BD Biosciences, Franklin Lakes, New Jersey, USA) and data were analyzed using FlowJo software (v10®, Ashland, Oregon, USA).

For TCR sequencing experiments, some dominant CD8+ TCR Vβ+ cells were sorted on a FACSARIA IIu device (BD Biosciences).

Mass Cytometry Analysis

Mass cytometry antibodies were obtained as pre-conjugated metal-tagged antibodies from Fluidigm (South San Francisco, California, USA) or generated in-house by conjugating unlabelled purified antibodies (from Myltenyi or Beckman Coulter) to isotope-loaded polymers using Maxpar X8 Multi-Metal Labelling Kit (Fluidigm). After titration on Nanodrop ND 1000 (ThermoFischer) antibodies were diluted in antibody stabilization buffer (Candor-Biosciences, Wangen im Allgäu, Germany) with 0.5% sodium azide (Sigma). A detailed list of the antibodies used in this study is provided in supplementary materials (Table S2). Cell identification was performed using Irridium-Intercalator (Fluidigm) and viability discrimination was assessed by staining cells with Cisplatin (194Pt, Fluidigm). In some experiments, cells were fixed and permeabilized using Cytofix/Cytoperm solution (Cytofix/Cytoperm™, BD Biosciences, Le Pont de Claix, France) and next intra-cellularly stained with human anti-Granulysin, anti-Granzyme A, anti-Granzyme B, and anti-Perforin mAbs.

Before acquisition on HELIOS mass cytometer (Fluidigm) cells were resuspended in half-diluted Four Element Calibration Beads (Fluidigm), and data set were normalized with CyTOF software using Finck algorithm (47). Flow Cytometry Standard (FCS) 3.0 files were imported into FlowJo software v10®, and analyses included standard gating to remove beads, aggregates or dead cells, and further identify main leucocyte subsets (data not shown).

High-Dimensional Mass Cytometry Data Analysis

An inverse hyperbolic sine transformation was applied to analyze TCR αβ+ CD8+ T cells (n=300 per samples, all CyTOF samples were used (Table 1), except skin samples from MPE-9 and -12, which were excluded from the analysis due to very low CD8+ T cell number, and TEN-18 samples due to technical problem). Data were next clustered using FlowSOM algorithm (48) (with FlowSOM R pluging downloaded in FlowJo v10). A self-organizing map (SOM) was first trained to gather all cells into 100 distinct nodes based on their similarities in high dimensional space (i.e considering the relative MFI of 16 markers simultaneously: CCR7, CD45RA, CD27, CD38, CD56, CD57, CD107a, CD137, CD226, CD253, CD255, Granzyme A, Granzyme B, Granulysin, Perforin, Annexin A1, and excluding cell-lineage: CD45, CD14, CD19, TCRαβ, TCRγδ, CD8a, CD8b, CD4, CD38, CD56, NKP46, CD11b, CD11c, TCRVα14-Ja18, TCRVα7.2. SOM nodes were subsequently grouped in different clusters (each representing different CD8+ T cell subsets) using K-parameter and/or K-Finder R package (https://arxiv.org/pdf/1811.07356.pdf) (based on the Tracy Widom algorithm to approximate ‘K’ in sparse data matrices, ‘K’ being the number of relevant clusters in a population). FlowSOM clusters were visualized as integrated (i.e. including all samples) or disease phenotype minimal spanning trees, and heatmaps showing the integrated or individual MFI of each marker per cluster were generated with FlowJo or Excel. Additional hierarchical metaclusterings were performed, using the gplots R package based on the Euclidean distance and Ward-linkage (49), to determine the immunophenotype or the frequency of each cluster per samples.

DNA Isolation and High-Throughput Sequencing of TCRa/b CD3R Regions

DNA was isolated from frozen total blister, skin and PBMC samples using QIAamp DNA Micro Kit (Qiagen, Courtaboeuf, France), according to manufacturer's instructions. Then, TRB and TRA CDR3 regions were amplified and sequenced using ImmunoSEQ assay (Adaptive Biotechnologies). In brief, bias-controlled V and J gene primers were used to amplify rearranged V(D)J segments spanning each unique CDR3b/a, and amplicons were next sequenced (at approx. 20× coverage) using the Illumina HiSeq platform. The assay was performed at survey level (detection sensitivity: 1 cell in 40,000). After correcting sequencing errors via a clustering algorithm, TCRβ/α V, D and J genes were annotated according to the IMGT database (http://www.imgt.org).

Sequencing data were analyzed according to the ImmunoSEQ Analyzer V.3.0 (http://www.immunoseq.com). Diverse TCR repertoire metrics were explored: frequency and overlap of highly expanded clones, respective nucleotide or amino acid CDR3 sequences, usage and pairing of TRB/AV, TRBD and TRB/AJ families or diversity of the TCR repertoire (clonality index based on Shannon's entropy).

Transduction of the Vα- and Vβ-Chains of the TCR into Skw3 Cell Lines

Skw3 cell lines (Leibniz Institute DSMZ, Brunswick, Germany, (50)) were transduced as described previously (51). In brief, rearranged human variable TCR α- and β genes identified by TCR sequencing were synthesized by custom gene synthesis (GeneUniversal, Newark, Delaware, USA) and cloned into retroviral pMSCV Vector (Takara Bio USA Inc, Mountain View, California, USA) containing puromycin and neomycin resistance genes respectively. The resulting retroviruses were used to transduce the TCR-defective Skw3 cell line, which also expresses the human CD8 coreceptor. The TCR-transduced cells outgrowing in selective medium were picked, and the expression of the correct TCR α and β was further assessed by flow cytometry, using a FACS-Canto-I device (BD-Biosciences, San Jose, CA, USA). The transduced cells with stable TCR expression were selected for assessment of reactivity and specificity, which was measured by TCR-induced CD69 expression.

TCR-Transductant Stimulation Assay

Skw3 cell lines expressing the cognate TCR α and β chains were cocultured with autologous EBV-transformed B-lymphoblastoid cell lines (52) at 1:2 ratio at 37° C. Tested drugs were added to the cocultures with the indicated concentrations. After 24 h, cells were stained with anti-human CD3 (Biolegend) and anti-human CD69 (Biolegend) and analysed by flow cytometry. Levels of CD69 expression were monitored in 10,000 CD3⁺ events. Experiments were repeated at least 2 times.

Statistical Analysis

P-values were calculated with two-tailed independent Student's t tests or one-way analysis of variance (ANOVA) using GraphPad Prism software (v8®, San Diego, California, USA). P-values <0.05 were considered significant.

The Tukey's rule for the detection of outliers (75th percentile (Q3)+1.5× inter-quartile range (IQR)) was used to identify highly expanded TCR Vβ chains. Of note, all TEN, MPE and healthy donor data for each Vβ chain were compiled to calculate IQR.

Results

Skin and blood samples were collected from 18 TEN and 14 MPE hospitalized patients at the acute phase of their disease. Samples were recovered within 0-2 days after their hospital admission and diagnosis, and within 0-5 days after the first symptoms (mainly fever and/or skin rash). Hence, majority of samples were collected before the peak of the disease, characterized for TEN patients by the maximal percentage of skin detachment (Table 1 & data not shown). Noteworthy, the majority of patients displayed very diverse HLA genotypes. A*02 and B*44 were the most represented loci (Table 1). A careful investigation of causative drug(s) associated to skin symptoms revealed a large variability in terms of drug nature or mode of action. The same molecule was reported as culprit drug only for pairs of TEN patients (Allopurinol for patients TEN-1 & -3; Sulfamethozaxole/Trimethoprim for TEN-2 & -5; and Ceftriaxone for TEN-10 & -11, (Table 1)).

Immunophenotype of Leukocytes Infiltrating the Skin of TEN Patients

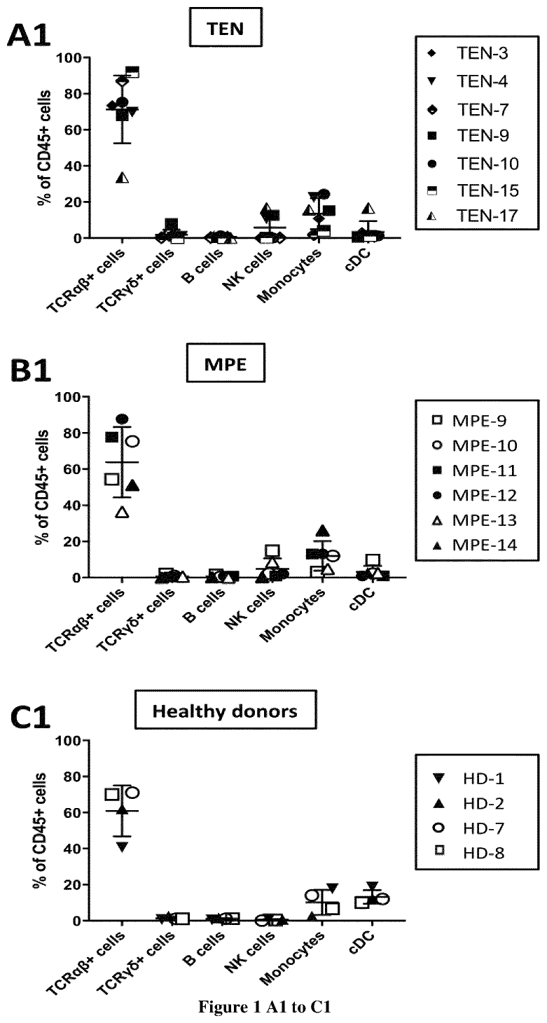

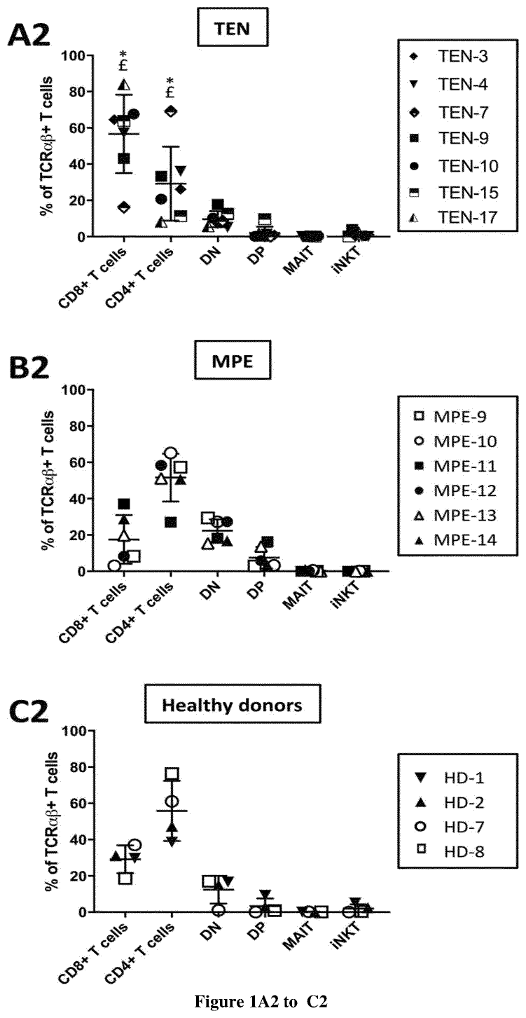

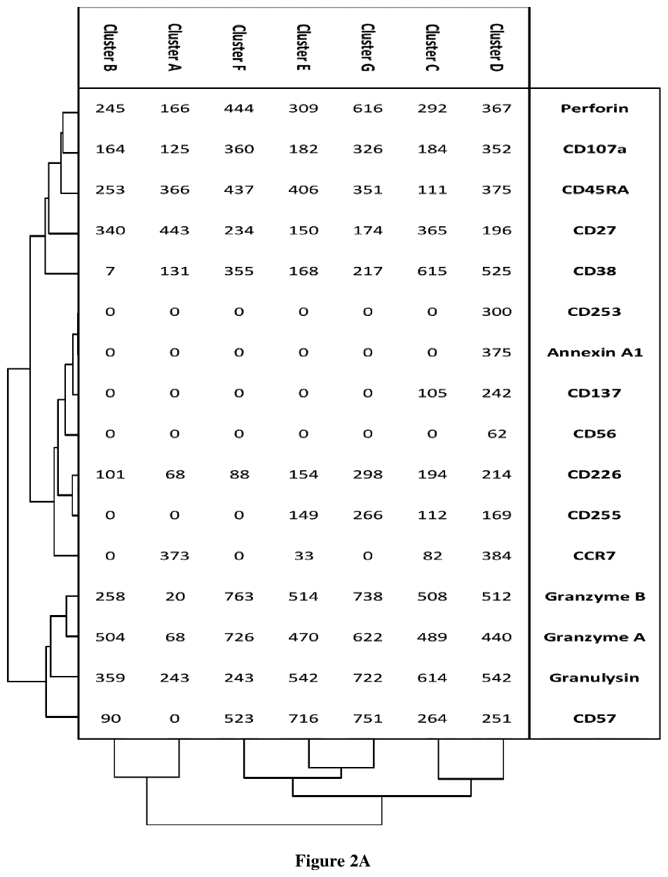

We first examined the immunophenotype of cells infiltrating the skin of TEN patients by mass cytometry (cyTOF) and subsequent computational data analysis. Blister cell samples obtained from 7 TEN patients were analyzed by CyTOF using a panel of 29 antibodies (Table S2), enabling mapping of all major peripheral blood mononuclear cell subsets (data not shown). We detected a large predominance of conventional T lymphocytes (TCRαβ+; mean±standard deviation (SD)=71.3±18.8%) among hematopoietic CD45+ cells, along with a minor infiltration of monocytes (CD14+ subset, 13.47±8.6%), NK (TCRαβ-CD56+, 5.8±7.2%) cells, and very few gamma delta T (TCRγδ+, 1.9±2.8%), B (CD19+, 0.6±0.6%) or dendritic cells (CD11c+, 3.4±5.9%) (FIG. 1, A1). Conventional T lymphocytes were CD8+ (56.64±21.6%), CD4+ (29.24±20.4%) or double-negative (DN) (9.6±4.4%) T cells (FIG. 1, A2), and rare positive (DP, 2.0±3.4%), MAIT (CD4-CD8b-TCRVa7.2+, 0.2±0.1%) or invariant NKT (iNKT; TCRαβ^{int} TCRVα24+, 1.0±1.5%) cells were recorded for all the patients (FIG. 1, A2). When adjacent skin biopsies were collected, instead of blister fluids, similar results were found, except for an increased representation of CD4+ versus CD8+ fraction cells (data not shown).

Similarly to TEN, the inflamed skin of MPE patients was infiltrated by conventional T lymphocytes (63.8±19.5% of hematopoietic CD45+ cells), and to a lesser extent, by CD14+ monocytes (12.3±8.1%) and NK cells (4.8±5.8%) (FIG. 1, B1). In contrast to TEN, the CD4+ fraction (51.58±13.2%) was greater than the CD8+ counterpart (17.6%±13.4) (FIG. 1, B2). These frequencies were comparable to those found in the skin of healthy donors (FIG. 1, C1-C2).

Finally, we detected no major difference in the immunophenotype of cells circulating in the blood of TEN, MPE patients and healthy donors, with CD8+ T cells representing approximately a quarter of total TCRαβ+ cells in all the tested samples (data not shown).

Collectively, these results thus confirm that the blistering and inflamed skin of TEN patients is extensively infiltrated by CD8+ T cells (14) (26) (20). By contrast, no major skewing was recorded for unconventional lymphocytes, NK cells or monocytes.

Clustering of Skin CD8+ T Cells into 7 Phenotypic FlowSOM Subsets

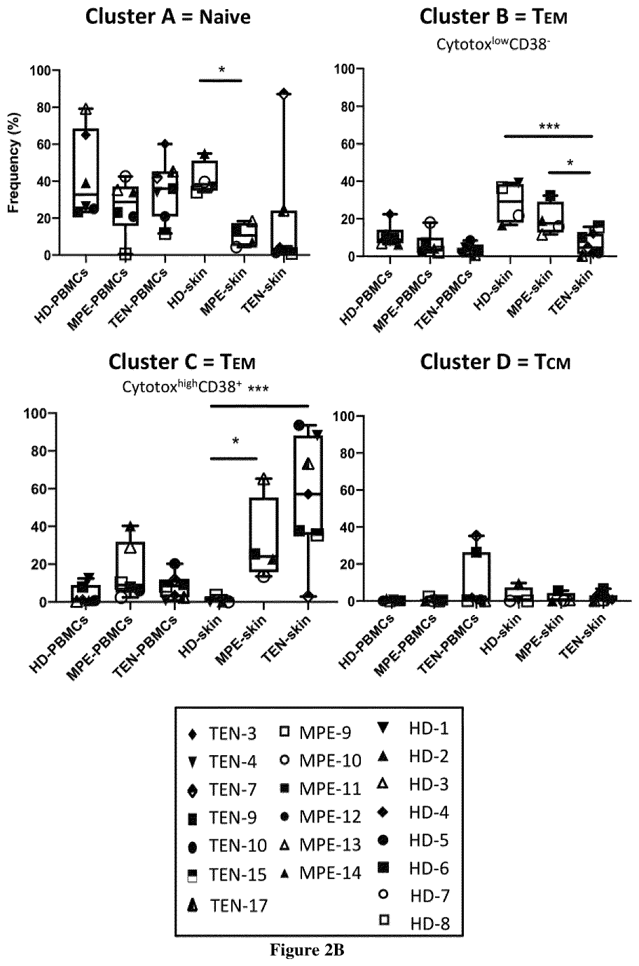

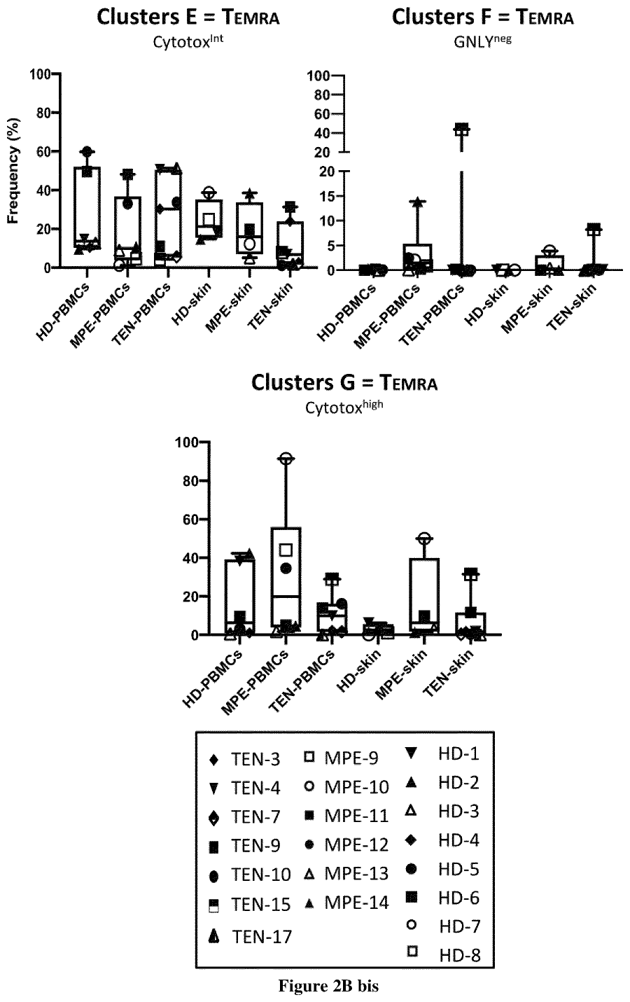

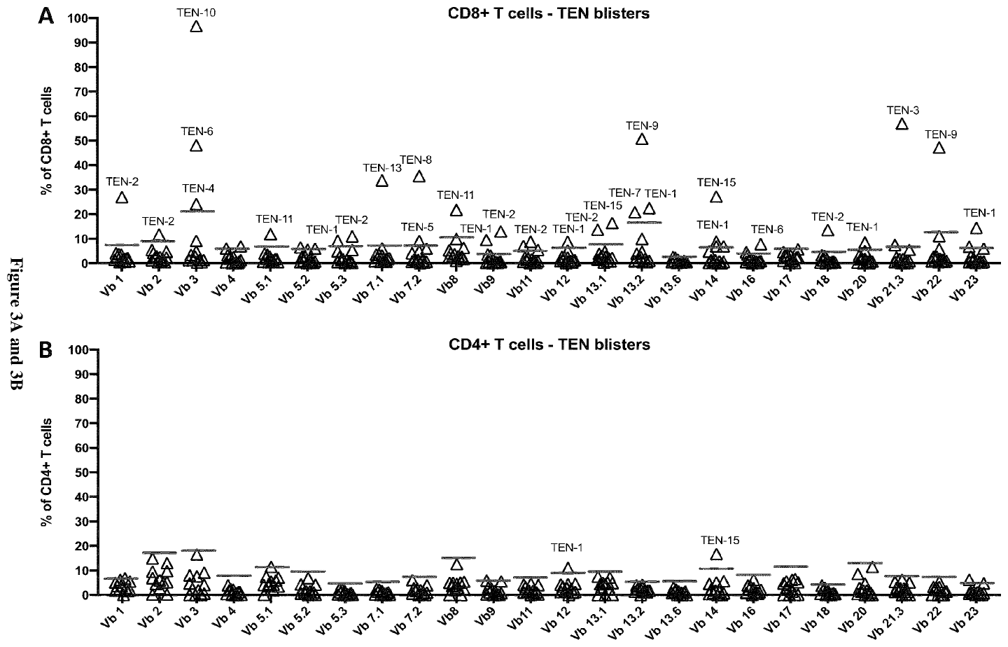

As CD8+ T cell-mediated cytotoxicity is key in the initiation and formation of drug-induced lesions, we investigated in detail the molecular cytotoxic expression patterns of CD8+ T cells in TEN blisters. We performed high dimensional profiling and investigated the (co-)expression of several cell death-associated molecules (Granulysin, Granzyme B, Granzyme A, Perforin, but also TRAIL (CD253), TWEAK (CD255), Annexin A1, CD107a), as well as different activation markers (CD27, CD38, CD56, CD57, CD137, CD226). Using concatenated CyTOF data from different samples (skin and peripheral blood mononuclear cells (PBMCs) from TEN, MPE but also healthy donors), we ran FlowSOM, a self-organizing map (SOM) clustering algorithm, to assess the heterogeneity of the CD8+ T cell population present in the different patients. FlowSOM first stratified the CD8+ T cell population into 100 nodes. Projected as minimal spanning tree (data not shown), each SOM node groups cells with similar phenotypes, with the node size representing the number of cells within that node (illustrations of minimal spanning tree obtained for each tissue sample are also shown (data not shown). SOM nodes were next gathered in 4 main clusters, as automatically calculated using K-finder Tree-level approach algorithm. Because K-finder approach did not capture the full diversity of the concatenated population (data not shown), we decided to increase the FlowSOM clustering to 7 distinct clusters (clusters A to G). To define the phenotype identity of each cluster, we generated a heatmap showing the integrated median fluorescence intensity (iMFI) values of each marker in each FlowSOM cluster (FIG. 2A). Cluster A displayed a phenotypic identity coincident with naïve T cells (characterized by high levels of CD45RA, CCR7 or CD27, and by the lack of classical cytotoxic markers such as Granulysin, Granzyme B, Granzyme A or Perforin), while clusters E, F and G recapitulated the main features of TEMRA (effector memory T cell re-expressing CD45RA) cells, i.e. high levels of CD45RA, CD57 and low levels of CCR7, and with Granzyme A, Granzyme B, Perforin and Granulysin as main variables between clusters (moderate and high cytotoxicity, respectively for clusters E, F and G, but with no Granulysin expression in cluster F) (FIG. 2A & data not shown). Alternatively, clusters B and C both displayed a phenotype of effector memory lymphocytes (TEM; CCR7−, CD45RA−), but conversely to the former, cluster C was characterized by a phenotype of activated cytotoxic cells, as illustrated by their high level of CD38, Granzyme B and Granulysin expression (FIG. 2A & data not shown). The cluster D subpopulation bore some of the hallmarks of central memory T cells (TCM; CCR7⁺, CD45RA⁻), and also an elevated expression of CD38, Annexin A1 and CD253 markers (FIG. 2A& data not shown).

A Polycytotoxic Signature Typifies Lesional CD8+ TEN T Cells

The in-depth FlowSOM analysis allowed a comparison of the frequency of the CD8+ T cell clusters in lesion (blisters and skin) and blood samples from TEN and MPE patients, and from healthy individuals (FIG. 2B). Most of the clusters were present in all patient samples, except for clusters D and F found only in a few. A degree of inter-individual variation was found only in 2 and 3 patients respectively. Notably, the activated polycytotoxic effector memory subset (cluster C) was consistently elevated in TEN (mean: 55% of infiltrating CD8+ T cells) and to a lesser extent in MPE (mean: 30%) skin samples, relative to healthy donor (mean: 1%) samples (FIG. 2B). Unlike the other clusters, cluster C expressed high levels of the cell surface activation marker CD38.

These results thus establish that the major subset of TEN blister CD8+ T cells displays a hallmark CD38+ polycytotoxic effector memory cell phenotype (cluster C).

Restricted TCR Vβ Repertoire Among TEN Blister and Blood CD8+ T Cells

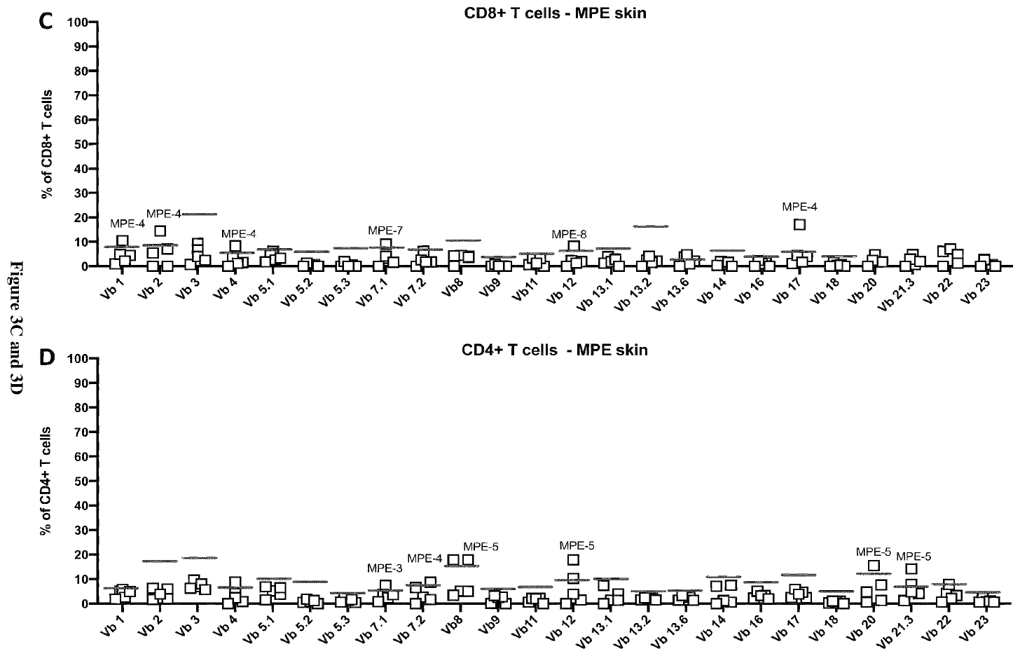

Parallel to these studies, we also addressed TCR usage of T cells present in TEN blisters. FACS analysis conducted on 24 of the most common Vbeta (Vβ) chains found a highly restricted TCRVβ repertoire usage in the 13 TEN patients tested, with single Vβ expansions ranging from as much as 20% to 80% of total TCR-Vβ chains expression, when compared to healthy donors (FIG. 3 & data not shown). This preferential usage, detectable at the CD3+ population level (data not shown), concerned almost exclusively CD8+ (FIG. 3A) and rarely CD4+ T cells (FIG. 3B). It concerned quasi all the 24 Vβ chains (with the exception of Vβ4, Vβ5.2, Vβ13.6 and Vβ17, using antibody Vβ nomenclature). Vβ3 and Vβ13.2 were the most overrepresented Vβ chains, each found in 3/13 of TEN patients. TEN-1 and TEN-2 patients showed overexpression of at least 6 TCR-Vβ chains, and TEN-9 exhibited 2 dominant Vβ13.2+ and Vβ22+ chains, representing each approximately 45% of total TCR-Vβ repertoire for this patient (FIG. 3A).

Although less marked than in TEN blisters, TCR VD expansions were observed in CD8+ T cells (but not CD4+ T cells) from TEN PBMCs, with notable biases in patients TEN-3, -4, -5-6, -10, -11, -13 and -15 (data not shown). In contrast, a limited number of TCR Vβ expansions were detected in CD8+ and CD4+ T cells isolated from MPE skin (FIGS. 3C & 3D and data not shown) and PBMC samples, when compared to healthy donors (data not shown).

Massive Oligoclonal Expansion of Distinct CDR3 Clones in the Skin and Blood of TEN Patients

As FACS cannot catch the full spectrum of the TCR repertoire, we next used high-throughput sequencing (HTS) of the TCR CDR3 regions (the antigen recognition domains) to evaluate sample clonality. HTS was performed on total blister, skin and PBMC samples from TEN and MPE patients.

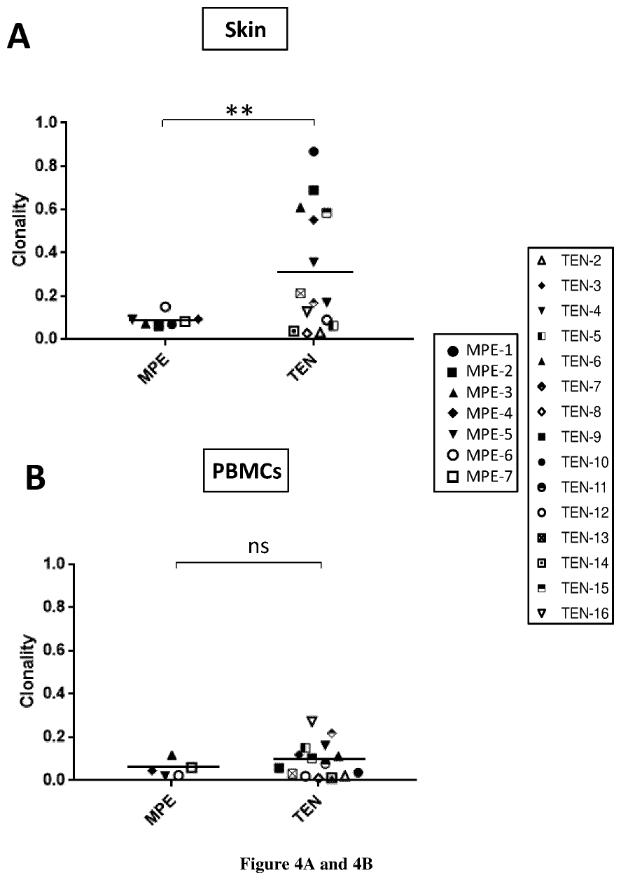

Investigations of TCR repertoire diversity, measured using Shanon entropy-based clonality index metric, first revealed the presence of a highly clonal repertoire in the blisters of approximately half of TEN patients (FIG. 4A & data not shown). By contrast, no difference was detected among PBMCs from TEN and MPE patients (FIG. 4B & data not shown) compared to healthy subjects (data not shown; for healthy donor comparison, data were retrieved from Adaptive Biotechnologies project on normal human PBMCs at https://www.adaptivebiotech.com/products-services/immunoseq/immunoseq-analyzer, and from (27), thus including data from 44 healthy donors).

In-depth analysis of TRBV repertoire next confirmed the existence of preferential TCR biases in the skin of 12 out of 15 TEN patients, which were the result of very limited numbers of CDR3 clonotype expansions (ranging from >10% to 90% of total TCR sequences for combined top 5 clones; except TEN-2, -8 and -14) (data not shown). Of note, clone-tracking analyses revealed (i) that expanded clones expressed the same VD chains cells as those observed by FACS (data not shown) and (ii) no sharing of identical TCR CDR3 nucleotide (data not shown) or amino acid (data not shown) sequences among the 15 TEN patients. An interesting exception was noted for one clone from patients TEN-6 and TEN-10, which shared amino acid but not nucleotide sequence (data not shown). As these patients were exposed respectively to Norfloxacin and Ciprofloxacin quinolones and both expressed HLA-B*73:03 (Table 1), potential epitope cross-reactivity expansion of clones sharing identical TCR beta chains.

Another interesting observation was noted for the 2 dominant clones from patient TEN-9 (FIG. 3). Parallel TRBV and TRAV investigations performed on FACS-sorted CD8+TCRVβ13.2+ and CD8+TCRVβ22+ cells of this patient, revealed that they represent in fact an unique T cell clone, which has rearranged two functional TCRbeta genes (respectively TRBV02-01*01 and TRBV06 sequences), as well as two functional TCRalpha genes (with the same TRVA19-01*01 sequence, but distinct TRAJ segments, respectively TRAJ30-01*01 and TRAJ29-01*01) (data not shown).

Conversely to TEN, similar TRBV repertoire analysis revealed that clonal expansions were rare for MPE patients, and were usually lower than 5% (data not shown).

Clonotypes that were massively expanded in the TEN blisters were also found elevated in the blood of respective patients, at least for the top 5 clones (data not shown). This result then indicates that the massive infiltration of unique clonotypes in TEN blisters was likely to be consecutive to a previous clonal expansion in the lymphoid organs. Only for patient TEN-15, and to a lesser extent for patients TEN-6 and TEN-11, were some of the highly expanded skin clones not represented in the blood (data not shown).

T Cell Repertoire Diversity and Clonal Expansion of Blister Clones Circulating in Blood Correlates with TEN Severity

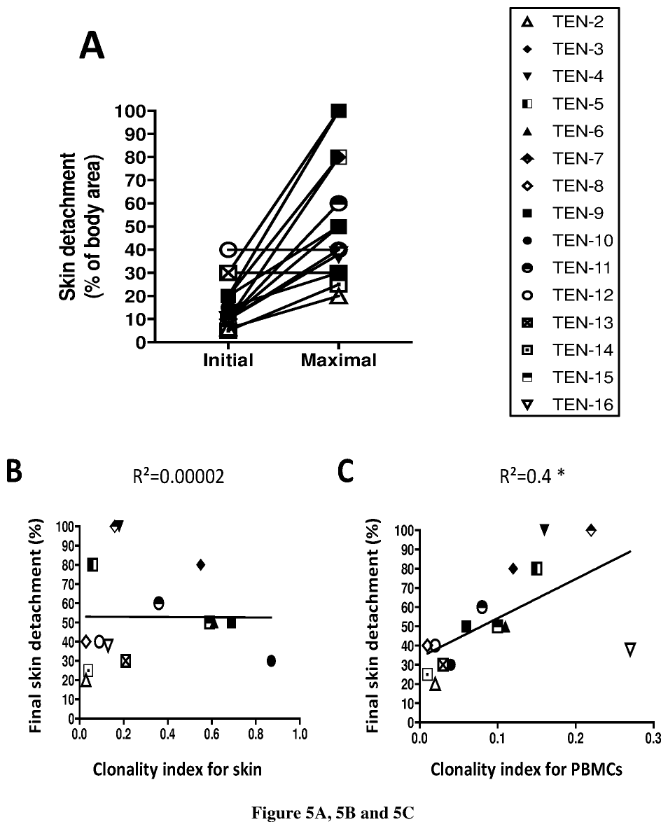

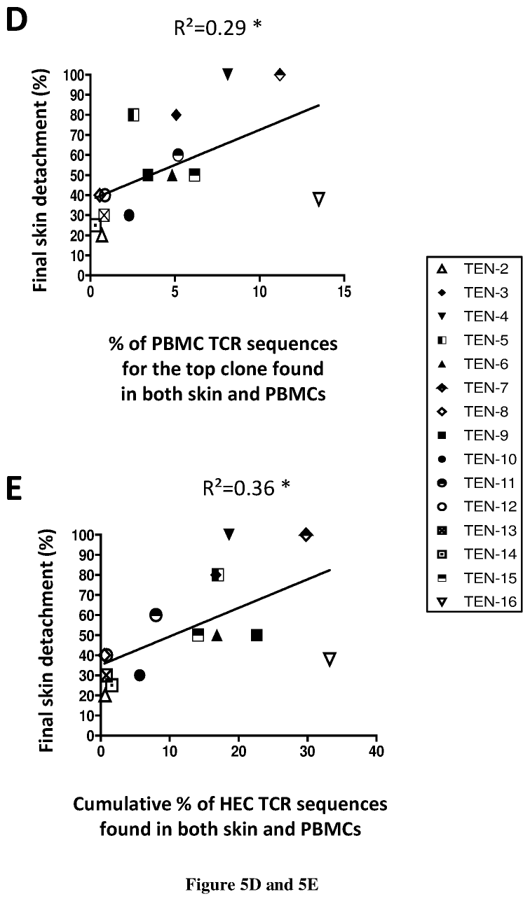

TEN severity, assessed here as the percentage of final skin detachment, varied significantly after hospital admission (FIG. 5A), and was maximal between 1 to 7 days (mean±SD=3.2±1.6 days) (Table 1). We then investigated potential correlations correlations existed between clonal expansions in the blisters or the blood of TEN patients (measured at days 0-2 after hospital admission) and final skin detachment. While no association was detected with blister clonality indices (R=0.00003, p=NS; FIG. 5B), by contrast, we observed that patients with the highest PBMC clonality indices presented the highest percentages of final skin detachment (R²=0.4, p=0.01; FIG. 5C). Besides, substantial correlations were also found between the percentage of top blister clones circulating in blood and the percentage of final skin detachment, as shown for top 1 (R²=0.29, p=0.04; FIG. 5D) and for the highly expanded clones (i.e. clones represented at a frequency >0.05% of TRBV repertoire in each patient; R²=0.36, p=0.02; data not shown & FIG. 5E).

Combined with the lack of major TCR CDR3 biases found in MPE samples (skin or blood) (FIGS. 4 & data not shown), our results thus demonstrate that the massive expansion of unique clonotypes is a major feature of TEN pathology and that the level of expansion of those unique clonotypes among PBMCs at acute phase is directly related to clinical severity.

Vβ-Expanded CD8+ T Cells Display the Polycytotoxic Phenotype Over-Represented in TEN Samples

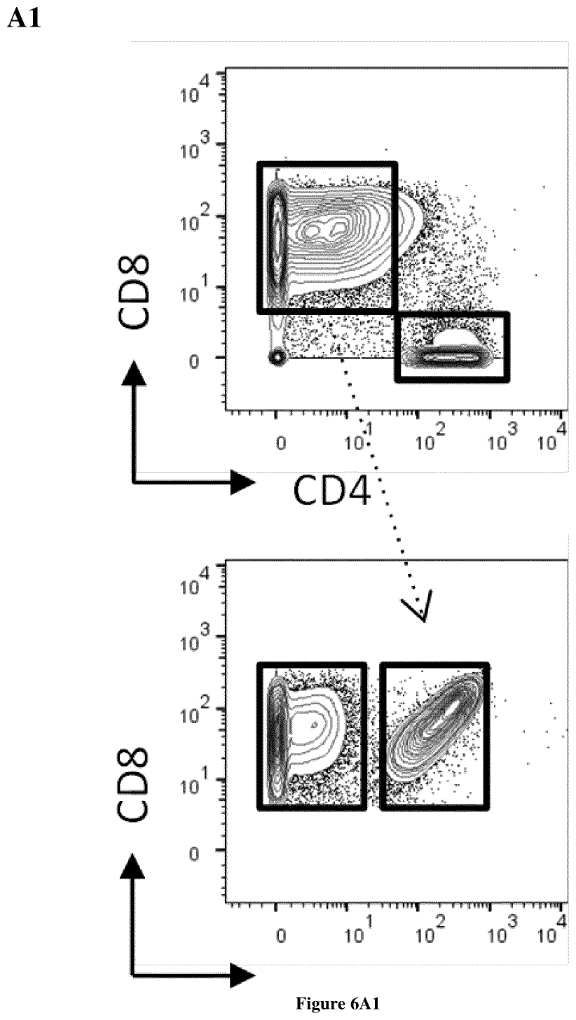

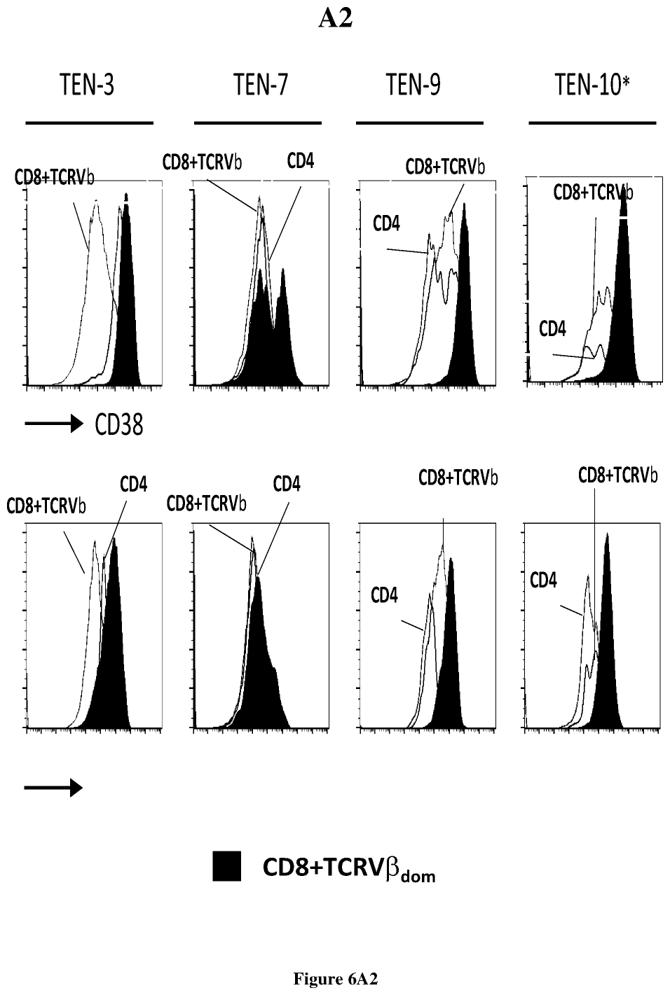

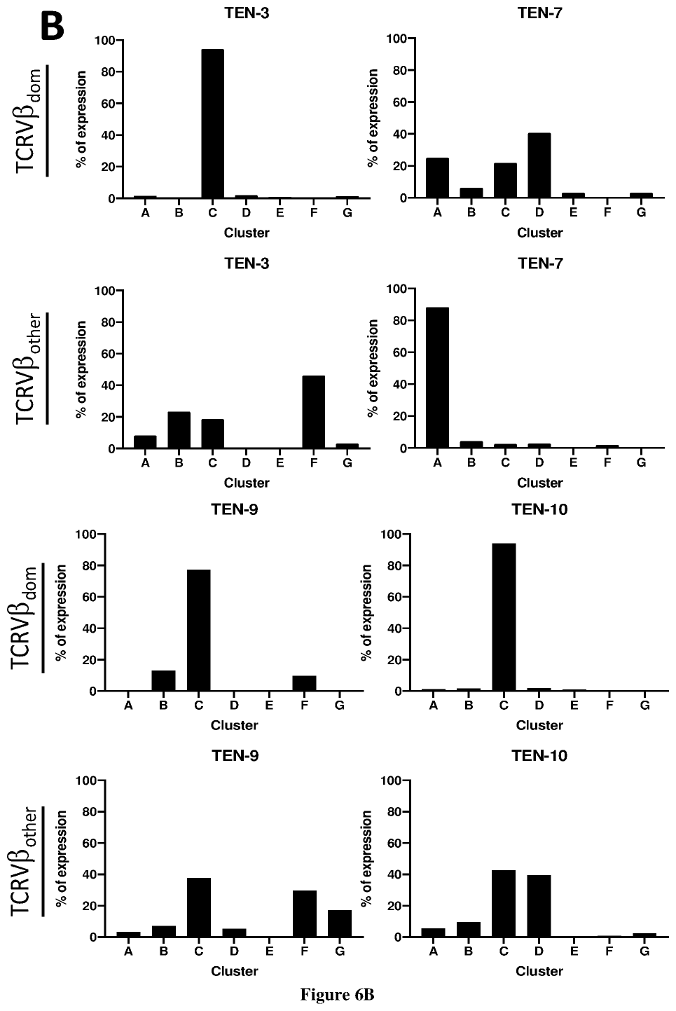

Thereafter, by taking advantage of mass cytometry, we were able to track back highly Vβ-expanded CD8+ T cells in the blisters and blood of TEN patients to analyse their phenotype. We first demonstrated that CD8+ T cells expressing dominant Vβ chains (FACS analysis) displayed very high levels of Granulysin and CD38 markers, when compared to their non-dominant CD8+Vβ+ T cell counterparts (FIG. 6A). By superimposing dominant and non-dominant TCRVβ+ markers on our concatenated CD8+ T cell clusters, we next demonstrated that skin dominant TCRVβ+ cells mainly expressed the cytotoxic cluster C phenotype (FIG. 6B). Conversely, the non-dominant TCRV β+ cells were detected in all the different clusters.

This analysis confirms that major Vβ-expanded CD8+ T cells display the polycytotoxic phenotype that is over-represented in TEN samples.

Expanded Clones in Blisters and Blood are Drug-Specific

Ultimately, we sought to determine whether highly expanded and activated clones were drug specific. To this end, we FACS sorted dominant CD8+ TCRVβ+ T cells present in the blister fluids or the blood of 4 TEN patients (TEN-3,-7,-10,-15), and sequenced their TRAV repertoire. For most dominant clones, a productive rearrangement (data not shown) encoding a functional TCR alpha-chain, as well as a second non-productive TCR alpha-locus rearrangement (data not shown) were identified. Then, the productive rearranged TCRα and TCRβ chains were transduced into Skw3 cells, a TCR defective lymphoma line (28) (data not shown). After verification of sustained and stable TCR expression (data not shown), transduced Skw3 cells were stimulated with the culprit (or control) drug in presence of autologous Epstein Barr Virus (EBV)-transformed B cells generated from patient's PBMCs. The following day, we measured CD69 expression at the surface of Skw3 cells, as marker for TCR stimulation.

Results showed a positive dose response for patient TEN-3 with oxypurinol (the metabolite of allopurinol, the culprit drug for TEN-3), but not with the parent drug or an irrelevant drug (sulfamethoxazole) (Table 2 & data not shown). A positive response was also found for patient TEN-7 with the culprit pantoprazole (Table 2). In contrast, we failed to detect robust CD69+ expression in transductants generated from patients TEN-10 and TEN-15, stimulated respectively with ceftriaxone and ciprofloxacin or levofloxacin and metronidazole (Table 2).

### DISCUSSION

The main objective of our study was to gain further insights into TEN pathophysiology by tracking immune cells that are present in the skin and the blood of patients at disease onset. Our results confirm that CTLs are the main leucocyte subset found in TEN blisters, followed by a minor infiltration of CD14+ monocytes and NK cells; but we failed to repeatedly detect unconventional cytotoxic lymphocytes such as NKT, MAIT or gamma-delta T cells. Strikingly, deep sequencing of the T cell receptor CDR3 repertoire revealed that there was a massive expansion of unique CD8+ T cell clones in TEN patients (both in skin and blood), which express an effector memory phenotype and an elevated level of cytotoxic or inflammatory/activation markers such as Granulysin, Granzymes A & B or CD38. By transducing α and β chains of the expanded clones into immortalized T cells, we demonstrate that some of these clones were drug-specific. Importantly, T cell repertoire diversity analysis revealed that clonal expansion of blister clones circulating in the blood of TEN patients at the acute phase of the disease correlated with the final clinical severity (as defined by the maximal percentage of skin detachment).

Massive Expansion of Unique TCR Clonotypes in TEN Patients

The most striking observation of our study is certainly the demonstration that there is a dramatic expansion of unique polycytotoxic CD8+ T cell clones in TEN patients, which largely outnumbers the frequency of clonotypes expanding in less severe MPE patients. A few studies have already described oligoclonal expansion in TEN (or in the less severe Stevens-Johnson syndrome (SJS)). These studies focused on in vitro T cell (re)activation experiments, or used samples which were isolated from individuals with restricted HLA genotype (for instance HLA-B*15.02 ((4) (29)) and reactive to a limited number of compounds (mainly allopurinol and carbamazepine) (4) (30) (29) (31). They showed preferential usage of TRBV subtypes, clonal expansion of specific CDR3 and less TCR diversity, in comparison to data obtained from healthy or drug-tolerant donors. Similarly, the infiltration of predominant T cell clones has already been reported in many benign inflammatory skin diseases such as psoriasis, atopic dermatitis and contact dermatitis (32) (33) (and in MPE, as shown in our study (data not shown)). Here, novelty then resides in the demonstration that the strength of clonal expansions reached levels (both in blisters and blood) that have only been described in skin neoplasic disorders, such as cutaneous T cell lymphomas (CTCLs) (33). Additionally, the fact that our results can be generalized to patients expressing highly diverse HLA genotypes and reactive to very different drugs (Table 1), thus reinforces the idea that a massive clonal bias is a major immunological hallmark of TEN disease. Of note, as expected, we failed to detect any shared TCR sequences in our HLA diverse cohort, except for patients TEN-6 and TEN-10, exposed respectively to Norfloxacin and Ciprofloxacin quinolones, and who both expressed HLA-B*73:03 (unfortunately, due to low sampling, it was not possible to compare TCR sequences from TEN-1 & TEN-3 patients, harbouring the HLA-B*58 risk allele and exposed to allopurinol).

It will then be crucial to determine in the future the reasons for such clonal expansion in TEN disease compared to less severe MPE. (i) The massive production of inflammatory mediators noticed in the sera and the blister fluid of TEN patients (14) (34), or the reported defective Treg functions (34), certainly participates to enlarge the proliferation of drug-specific cells, but whether it is a consequence, a cause or both remains to be clarified. (ii) T regulatory cells (Treg) are critical regulators of CTLs causing TEN in mouse models (35). In this context, the reported defective functions of TEN circulating Tregs as well as their decreased ability to infiltrate the skin (36) (37), may explain the uncontrolled expansion and skin migration of drug specific CTLs. Interestingly, our data showed a differing CD4/CD8 ratio between TEN (ratio=0.5) and MPE skin (ratio=2) with MPE having a ratio similar to healthy skin (FIG. 1). This suggests that the skin CD4+ Tregs/CD8+ CTLs ratio may be a major parameter to control CTL activation in situ, and therefore disease progression in TEN versus MPE. Future studies are needed to confirm the quantitative and qualitative defects of skin Tregs in TEN compared to MPE. (iii) Alternatively, it could be hypothesized that TEN patients possess a drug-specific preimmune repertoire that is prone to considerable enlargement. Several preclinical studies have shown that the breadth of immune response strongly depends on the number of specific T cell precursors (38), and a recent study from Pan et al. (29) showed an expansion of public TCRβ clonotypes in single HLA-restricted carbamazepine allergic SJS/TEN patients, questioning the possibility that TEN patients with similar HLA and exposed to the same drug develop/amplify the same pathogenic T cell repertoire. (iv) Another assumption addresses heterologous immunity, and a possible accumulation of pathogenic clones due to cross-reactivity with a reservoir of virus-specific memory T cells (39). (v) Finally, it is still completely unknown whether drug accumulation (due to defective drug detoxification mechanisms (40) predominates within TEN, fostering continuous T cell stimulation.

Immunophenotype of TCR Clonotypes in TEN Patients

Another important point of the present study is the extended characterization of the expanded clonotypes, which mostly comprise CD8+ T cells endowed with a polycytotoxic phenotype. We observed that the dominant skin TCRVβ+ CTLs mainly expressed the cluster C phenotype, which was assigned to T_{EM} cells. As expected (40) (26), this subset expressed high levels of Granzyme A, Granzyme B and especially Granulysin markers, and it was the only subset (with cluster D, poorly represented in skin samples) to express the CD38 protein, which is classically associated with T cell activation and/or diapedesis (41). By contrast, it lacked the expression of the senescence marker CD57 (classically assigned to T_{EMRA} subsets), indicating that the expanded CTL clones correspond to recently activated T cells.

By comparison, CD8+ T cells infiltrating the skin of MPE or healthy donors displayed a distinct functional phenotype, as shown both at the total population level (data not shown) and after multidimensional analysis (FIG. 2). We notably detected less (MPE) or no (healthy donors) cluster C subset, but more non activated T_{EM} (cluster B), and a T_{EMRA} subset (cluster E) endowed with moderate expression of cytotoxic markers (when compared to other T_{EMRA} subsets). It is probable than the main differences recorded between TEN and MPE (notably the differing CD4:CD8 ratio, FIG. 1) are due to the strong clonotype expansions, and not to the different type of tissues we collected (TEN blister versus MPE skin), because comparative analysis of adjacent blister skin in TEN patients exhibited similar Vβ expansion and phenotype (data not shown). It will be interesting to determine in future studies whether drug-specific skin MPE T cells are also found in cluster C, as for TEN (20) (26). Besides, it will be important to uncover whether drug-specific T cells from TEN patients possess unique ability to expand and/or to differentiate into potent killer cells, when compared to MPE T cells. This challenging task might become feasible with T cell clones generated in vitro from precursors collected in TEN and MPE patients allergic to the same molecules.

Drug Specificity

A major finding of our study is the antigenic specificity of the highly expanded clones found in TEN patients. Indeed, we were able to demonstrate that some of our engineered transductants (produced from TEN-3 and TEN-7) responded to their putative culprit drugs in vitro. Interestingly, potential drug reactivity was also recorded with transductants, generated from the rearranged pairs of TCRbeta and TCRalpha genes detected in the unusual dominant clone found in patient TEN-9, who was exposed to multiple drugs (Table 1). Nevertheless, as no clear culprit drug was identified for this patient, it was not possible to validate the relevance of our findings (data not shown). In contrast, transductants generated from sequences identified in patients TEN-10 and TEN-15 failed to respond to the tested drugs (Ceftriaxone, Ciprofloxacin, Levofloxacin, Metronidazole; Table 2). Various reasons might explain these TEN-10 and TEN-15 results. The simplest hypothesis is that we did not transfect the appropriate pathogenic TCR sequences. Alternatively, in keeping with the results obtained with TEN-3 transductants, which confirmed that T cells from allopurinol allergic patients are reactive to its metabolite (oxypurinol), but not to the parent molecule (4), it is possible that our in vitro drug exposure conditions (during Skw3/EBV-transformed B cell cultures) did not generate enough metabolites or drug-induced epitopes necessary to activate the transductants (in particular for Ciprofloxaxin, Levofloxacin or Metronidazole). Similarly, we cannot exclude that a specific mode of drug-epitope presentation (using peculiar non-conventional HLA-presentation (42)) or the involvement of an altered peptide repertoire (12) (13), govern T cell expansion from patients TEN-10 or -15.

Correlation with Disease Severity

The identification of early biomarkers, which predict final severity, is a highly desirable goal to improve clinical management of TEN patients. Our data confirm and extend the recent study reported by Xiong et al., which compared TCR repertoire diversity in patients suffering from SJS or TEN and showed that TCR repertoire metrics correlate with disease severity (31). So far, it is still debated whether SJS is an early stage of TEN (SJS is a bullous cADRs characterized by <10% of skin detachment) or a different pathology (both at the etiological and mechanistic levels). Here, we enrolled patients with progressing but established TEN phenotype only (with 40-100% of skin detachment at the peak of disease; except for patient TEN-2 who displayed an SJS/TEN intermediate phenotype with 20% of skin detachment). Despite extensive clonal expansion in TEN blisters at disease onset, we failed to detect any correlation between blister TCR repertoire diversity (or the percentage of top skin clones, data not shown) and final skin severity (FIG. 5). However, the same clones were also highly expanded in TEN patients' blood, and the degree of their expansion in blood at the early phases of the disease showed significant correlation with the final disease severity (FIG. 6C-E), thus expanding the findings reported by Xiong et al. (30). This suggests that the progression and severity of the disease is directly linked to the quantity of pathogenic clones that circulate in the blood and are able to be recruited in the epidermis a few hours/days after. Hence, to track clonal expansions (or TCR repertoire diversity) at disease onset could prove of paramount value for clinicians who want to anticipate the evolution of this life-threatening disease, and develop adequate care measures. However, due to the low number of patients (n=15) tested in our TCR repertoire study, it will be crucial to validate our results with an extended cohort. Besides, it will be important to understand why there is no correlation with TCR repertoire metrics in the skin. The fact that we conducted this study on a prospective cohort with diverse HLA, reactive to different drugs of different half-lives and different pharmacological properties, suffering from different degrees of liver/kidney dysfunction, transferred for intensive care at different intervals after symptom onset, withdrawn with culprit drug at different time and treated with different molecules (Table 1), certainly explains the discrepancies between the extent of final skin detachment and clonal expansion in the blisters at the beginning of the disease. It will be therefore crucial to conduct future studies on a more controlled cohort to decipher the reasons for the strong blood but not skin correlation.

In conclusion, our results demonstrate that the quantity and quality of skin-recruited CTLs conditions the clinical presentation of cADRs. Importantly, they open major opportunities for the development of new prognostic markers in TEN.

TABLE 1
Patient demographics, clinical features and HLA genotype
(Part1)

| Demographics | | | Clinical Characterisitics | |
|---|---|---|---|---|
| Patient ID | Sex/Age | Ethnicity | Underlying diseases | Comorbidities |
| TEN-1 | M/48 | East Asian | Hyperuricemia | None |
| TEN-2 | M/39 | European | Urine tract infection | None |
| TEN-3 | F/40 | European | Hyperuricemia | None |
| TEN-4 | M/74 | European | Melanoma | None |
| TEN-5 | M/32 | North African | Pneumocystis | HIV+ |
| | | | prophylaxis | |
| TEN-6 | F/83 | European | Urine tract infection | Cardiac insufficiency |
| TEN-7 | M/50 | European | Gastritis | Cirrhosis |
| TEN-8 | F/33 | European | Bipolar disease | None |
| TEN-9 | F/34 | African | Chronic pain | None |
| | | American | | |
| TEN-10 | F/63 | European | Severe angina | None |
| TEN-11 | M/58 | European | Infectious osteoarthritis | Diabetes, renal insufficiency |
| TEN-12 | F/27 | European | Cirrhosis | Autoimmune hepatitis |
| TEN-13 | F/75 | European | Post-surgery infection | Bladder adenocarcinoma |
| TEN-14 | M/41 | European | Myeloma | None |
| TEN-15 | F/69 | European | Lung infection | Ischemic stroke, SLE |
| TEN-16 | F/69 | European | Lung Infection | None |
| TEN-17 | H/50 | European | Infection | None |
| TEN-18 | H/58 | European | Liver cancer | HCV+ |
| MPE-1 | M/18 | European | ENT infection | None |
| MPE-2 | M/61 | European | ENT infection | None |
| MPE-3 | F/68 | European | Breast infection | None |
| MPE-4 | F/78 | European | Myeloma | None |
| MPE-5 | F/71 | European | Cardiac insufficiency | None |
| MPE-6 | F/62 | European | Infectious osteoarthritis | None |
| MPE-7 | M/61 | North African | Pulmonary infection | None |
| MPE-8 | F/24 | East Asian | Chronic pain | None |
| MPE-9 | F/94 | European | Urine tract infection | None |
| MPE-10 | F/62 | European | Graft versus Host Disease | Bone marrow transplant |
| MPE-11 | F/39 | North African | Hypertension | SLE |
| MPE-12 | F/62 | European | Hypertension, Gout | None |
| MPE-13 | H/52 | North African | Myeloma | None |
| MPE-14 | F/67 | European | Dermatomyositis | None |

(Part 2)
Clinical Characterisitics

| | Drug exposure | | | % of skin | % & date of |
|---|---|---|---|---|---|
| | before onset | Date & nature of first | SCORTEN (TEN)/ | detachment | maximal skin |
| Culprit drug | (days) | symptoms | Severity (MPE) | at day 0 | detachment |
| Allopurinol | 8 | day-2/fever | 3 | 2% | 100% at day 2 |
| Sulfamethoxazole/Trimethoprim | 7 | day-2/fever | 1 | 6% | 20% at day 5 |
| Allopurinol | 15 | day-3/fever + skin rash | 2 | 20% | 80% at day 2 |
| Vemurafenib | 22 | day-4/skin rash | 5 | 30% | 100% at day 1 |
| Sulfamethoxazole/Trimethoprim | 15 | day-2/fever | 3 | 10% | 80% at day 2 |
| Norfloxacin | 8 | day-2/fever/skin rash | 3 | 20% | 50% at day 5 |
| Pantoprazole | 10 | day-1/fever + skin rash | 3 | 20% | 100% at day 2 |
| Lamotrigine | 12 | day-3/fever + eye stinging | 2 | 10% | 40% at day 5 |
| * | 2 | day-2/fever | 3 | 10% | 50% at day 3 |
| Ceftriaxone, Ciprofloxacin | 8 | day-4/skin rash | 2 | 15% | 30% at day 3 |
| Ceftriaxone | 15 | day-1/skin rash | 4 | 10% | 60% at day 2 |
| Furosemide | 21 | day-3/fever + skin rash | 3 | 40% | 40% at day 3 |
| Cefixime | 4 | day-1/fever + skin rash | 4 | 30% | 30% at day 2 |
| Revlimid | 15 | day-1 + fever + skin rash | 2 | 5% | 25% at day 3 |
| Levofloxacin, Metronidazole | 5 | day-2 + fever + skin rash | 3 | 10% | 50% at day 3 |
| Pristinamycin | 1 | day 0/fever + skin rash | 2 | 10% | 38% at day 2 |
| Azithromycin, paracetamol | 5 | day-2/fever + skin rash | 4 | 20% | 80% at day 5 |
| Sorafenib | 10 | day-3/skin rash | 5 | 5% | 48% at day 7 |
| Amoxicillin | 2 | day-1/skin rash | mild | na | na |
| Amoxicillin | 3 | day-1/skin rash | mild | na | na |
| Vancomycin | 28 | day-4/skin rash | severe | na | na |
| Bortezomid | 5 | day-4/skin rash | severe | na | na |
| Diltiazem | 15 | day-3/skin rash | severe | na | na |
| Vancomycin | 2 | day-1/skin rash | severe | na | na |
| Vancomycin | 42 | day-4/skin rash | mild | na | na |
| Ibuprofen | 9 | day-3/skin rash | severe | na | na |
| Clindamycin | 3 | day-3/skin rash | mild | na | na |
| Tazocillin, contrast material | 2 | day-1/skin rash | mild | na | na |
| Macrogol, Urapidil, Amlodipine | 14 | day-3/skin rash | moderate | na | na |
| Allopurinol, Fibrate | 28 | day-2/skin rash | mild | na | na |
| Revlimid, Bortezomid | 15 | day-2/skin rash | severe | na | na |
| Hydroxychloroquine | 15 | day-4/skin rash | mild | na | na |

(Part 3)

HLA genotype

| Treatment | Locus A | Locus B |
|---|---|---|
| Systemic corticosteroid + | A*02; A*33 | B*38; B*58 |
| G-CSF | A*30; A*30 | B*13; B*18 |
| | A*02; A*03 | B*27; B*58 |
| Maintenance of existing | A*03; A*23 | B*44; B*51 |
| corticosteroid therapy + | | |
| G-CSF | | |
| Systemic corticosteroid + | A*02; A*24 | B*44; B*45 |
| G-CSF | | |
| G-CSF | A*03; A*— | B*18; B*73: 01 |
| G-CSF | A*02; A*11 | B*15; B*44 |
| | A*02; A*30 | B*08; B*44 |
| G-CSF | A*02; A*02 | B*15; B*53 |
| | A*01: 03; A*68 | B*08; B*73: 01 |
| G-CSF | A*02; A*29 | B*44; B*45 |
| Maintenance of existing | A*01; A*— | B*08; B*51 |
| corticosteroid therapy + | | |
| G-CSF | | |
| G-CSF | A*02; A*— | B*44; B*57 |
| Systemic corticosteroid | A*02; A*02 | B*15; B*27 |
| | A*03; A*30 | B*18; B*40 |
| | A*02; A*03 | B*35; B*51 |
| G-CSF | A*02; A*03 | B*07; B*51 |
| G-CSF | A*03; A*11 | B*35; B*40 |
| Topical corticosteroid | A*01; A*02 | B*40; B*51 |
| Topical corticosteroid | A*02; A*— | B*08; B*40 |
| Topical corticosteroid | A*24; A*25 | B*15; B*18 |
| Topical corticosteroid | A*29; A*31 | B*35; B*44 |
| Topical corticosteroid | A*02; A*— | B*51; B*— |
| Topical corticosteroid | A*01; A*02 | B*40; B*57 |
| Topical corticosteroid | A*02; A*32 | B*49; B*51 |
| Topical corticosteroid | A*24; A*— | B*15; B*38 |
| Topical corticosteroid | A*23; A*31 | B*39; B49 |
| Topical corticosteroid | A*02; A*03 | B*15: 16; B*39 |
| Topical corticosteroid | A*32; A*34 | B*39; B*44 |
| Topical corticosteroid | A*23; A*68 | B*44; B53 |
| Systemic corticosteroid | A*01; A*02 | B*07; B*51 |
| Topical corticosteroid | A*01; A*29 | B*08; B*44 |

Alden's algorithm was used to determine culprit drugs for TEN patients. For MPE patients, the main putative drugs are also indicated.

Disease severity for TEN patients was evaluated by the SCORTEN at day 0 (arrival at hospital and diagnosis). The SCORTEN predicts the risk of death. The SCORTEN scale consists in 7 independent factors for high mortality, and varies from 0 or 1 (low mortality rate) to 5 or more (very high mortality rate). Disease severity was appreciated by calculating percentages of skin detachment (using E-Burn® application). The peak of disease was appreciated as the date at which TEN patients displayed maximal percentage of skin detachment.

Disease severity for MPE patients was estimated based on the extent of skin rash and the presence of systemic and/or visceral symptoms. None of the MPE patients exhibit symptoms suggestive of Drug Reaction and Eosinophilia Systemic Symptoms (DRESS)/Drug-Induced Hypersensitivity Syndrome (DIHS) and the Kardaun score was <3 for all the patients.

M=Male. F=Female. ENT=Ear Nose Throat. SLE=Systemic lupus erythematosus. HIV+=Human Immunodeficiency virus positive. HCV+=Hepatitis C virus positive, na=not applicable.

* no culprit drug was identified for patient TEN-9, using ALDEN algorithm. The patient received ibuprofen, doxycyclin, sulfamethoxazole-trimethoprime, tetracyclin, isoniazid, rifampicin in the days before TEN onset.

TABLE 2
Drug-induced activation of TCRαβ Skw3 transfectants

| | | | % of CD69 expression in CD3+ transfectants | | | |
|---|---|---|---|---|---|---|
| Patient ID | SKW3 transfectant ID | Drug concentrations (μg/ml)) | No drug | Concentration 1 | Concentration 2 | Concentration 3 |
| TEN-3 | C1 | Allopurinol (62.5/250) | 2.3 | | 2.2 | 3.05 |
| | | Oxypurinol (62.5/250) | 2.3 | | 22.9 | 39.6 |
| | | Sulfamethoxazole (100/200) | 2.3 | | 1.3 | 1.4 |
| TEN-7 | C2 | Pantoprazole (10/50) | 31.7 | | 40.1 | 47.4 |
| TEN-10 | C3 | Ceftriaxone (50/100/200) | 12.2 | 11.9 | 12.0 | 13.8 |
| | | Ciprofloxacin (12.5/25/50) | 10.9 | 10.1 | 11.8 | 10.6 |
| TEN-15 | C4 | Levofloxacin (25/50/100) | 6.0 | 5.6 | 5.4 | 4.4 |
| | | Metronidazole (25/50/100) | 6.3 | 5.7 | 5.4 | 6.0 |
| Control-1 | 17D | Abacavir (1/10/20) | 1.4 | 93.2 | 88.9 | 93.1 |
| | | Pantoprazole (12.5/25/50) | 1.4 | 1.8 | 1.8 | 1.7 |
| Control-2 | AnWe A1 | Allopurinol (62.5/250) | 4.3 | | 17.1 | 26.4 |
| | | Oxypurinol (62.5/250) | 4.3 | | 5.0 | 5.75 |
| Control-3 | UNO H13 | Ibuprofen (20/100/200) | 5.2 | 4.6 | 10.5 | 12.4 |

Skw3 cell lines engineered for the expression of TCRs bearing Vα and Vβ chains from top clones found in patients TEN-3,-7,-10 and -15 were stimulated in vitro with EBV-transformed B cells in presence of graded doses of different drugs, or left unpulsed. Table 2 depicts the percentage of CD69 expression in CD3+ transductants measured by FACS after 24 h stimulation. Results from control transductants generated from Abacavir- (17D), Allopurinol-(AnWeAl) or Sulfamethoxazole- (UNO H13) allergic donors (53) (51) (7) are also shown. Bold and underlined values indicate >2 or >1.5 CD69 expression fold increase versus unpulsed cultures.

Transductant ID are from Table S10.

Autologous EBV-transformed B cells were used for all the patients, except for patient TEN-7, for whom we did not have any autologous PBMCs available; hence we performed the same analysis with heterologous PBMCs from different healthy donors. Heterologous EBV-transformed B cells were also used to stimulate control transductants.

### Example 2 Preclinical Assessment of Anti-CD38 Monoclonal Antibody

The treatment of Toxic epidermal necrolysis (TEN), a rare but life-threatening cutaneous adverse drug reaction, is characterized by a rapidly progressing epidermal necrosis (1-2). TEN is associated with an important mortality rate of approximately 25-40%, and nearly constant and invalidating sequelae (blindness, respiratory disturbance . . . ), which are responsible for profound loss of quality of life in surviving patients.

To date, there is no efficient curative treatment for TEN, just palliative cares to relieve symptoms. TEN etiopathogenesis involves the recruitment and the activation into the skin of drug-specific polycytotoxic CD8+ T cells (3-6).

We have previously demonstrated that these cells express high levels of the activation marker CD38 (Example 1).

Goal and Method of the Therapeutic Approach

Therapeutic injection of anti-CD38 monoclonal antibody (mAb) to deplete the drug-specific cytotoxic CD8+CD38+ T cells as soon as the patient arrives to the clinic, in order to prevent/limit skin detachment and fatal outcome or invalidating sequelaes.

To make the proof of concept of the efficacy of anti-CD38 mAbs in a new TEN preclinical mouse model (i.e. humanized NGS mice transferred with CD8+ T cells collected from TEN patients at acute phase, and reactivated by the infusion of culprit drug(s)). We used this new preclinical model to assess the ability of a marketed anti-CD38 mAb, daratumumab, in depleting pathogenic cytotoxic CD8+CD38+ T cells.

Result in a Model of Graft Versus Host Versus Disease (GVHD).

We have assessed the efficacy of a marketed anti-CD38 mAb (daratumumab) to deplete human CD38+CD8+ T cells in a model of graft versus host versus disease (GVHD).

To this end, NGS mice were reconstituted with 10×10⁶ peripheral blood mononuclear cells (PBMCs) from a healthy donor, and treated by two-weekly injections of daratumumab (at 100 or 300 microg/mouse). Control group received PBS. Reconstitution is generally assessed by measuring the ratio of humanization (calculated by dividing the % of human blood CD45+ cells/the % of mouse blood CD45+ cells). A high ratio of humanization (>50-60%) classically correlates with the appearance of GVHD symptoms, approximately 1 month after transfer.

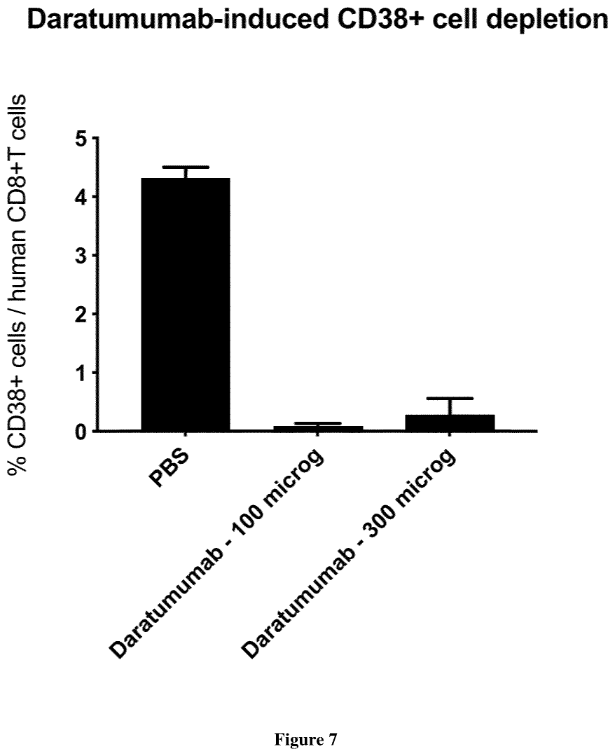

In preliminary experiments, we observed that daratumumab depleted CD38+ cells (FIG. 7), but failed to hamper the development of the GVHD at day 28 (not shown).

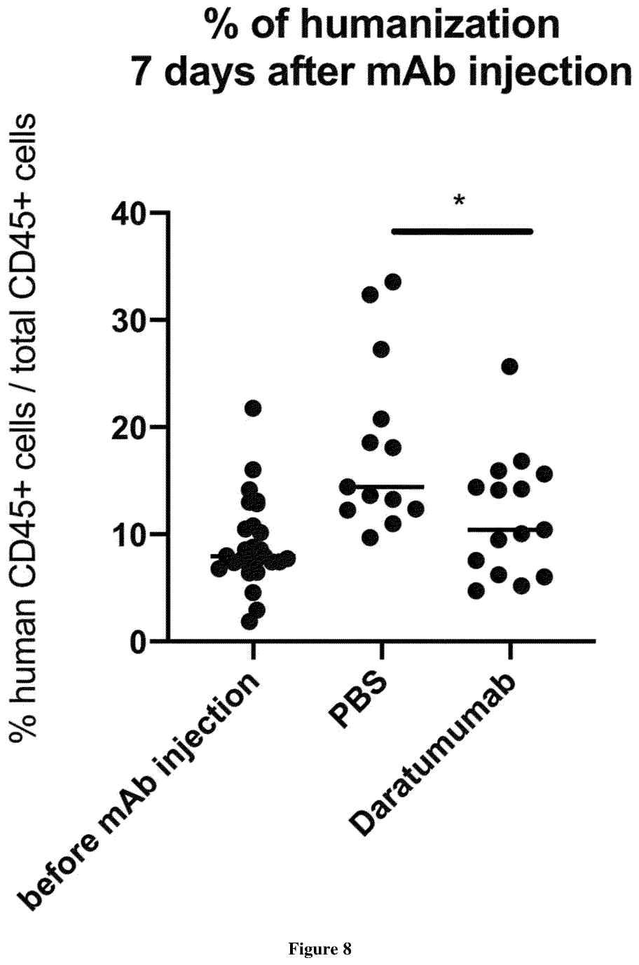

Nevertheless, interestingly, we recorded that daratumumab transiently inhibited the expansion of human cells (measured by calculating the ratio of humanization) at day 12 (that is 7 days after the initial daratumumab injection) (FIG. 8). This inhibition was lost at day 19 (that is 14 days after the initial daratumumab injection) (not shown).

Of note, higher daratumumab regimen (300 microg/mouse, 2 times a week) also failed to prevent GVDH development, but transiently inhibited T cell expansion (not shown).

Besides, FACS analysis demonstrated that expanding cells poorly expressed CD38 marker (approximately 5% of CD38+CD8+ T cells) at day 12 (FIG. 7).

It is probable that daratumumab failed to hamper GVDH development because pathogenic cells poorly expressed CD38+ in this model.

Therefore, to make the proof of concept of the efficacy of daratumumab, it is important to design a more relevant model, using CD38+ T cells collected from TEN patients.

Result in TEN Preclinical Model (NGS Mice).

Aims: (i) Determine engraftment upon cell transfer (CD8+ T cells isolated from TEN patients at acute phase). (ii) Characterize the immune response (lymphoid organs and peripheral tissues (skin, liver)), after drug administration. (iii) Characterize the clinical reaction: organ inflammation and cytokine production in the sera) induced by CD8+ T cell reactivation.

Deliverables: (i) Expansion of patient's cells before drug administration (i.e. percentage humanization=percentage of human versus mouse CD45+ cells). (ii) Percentage of proliferating (Ki67+) and activated (CD38+; Granulysine+ or Granzyme B+) CD8+ T cells in lymphoid organs, liver or skin. (iii) Skin, liver or kidney histology after drug administration. (iv) Main inflammatory cytokines/mediators in the sera (IL-1, IL-6, IL-15, TNF-α, IFNg, Granulysin, Granzyme B).

Proof of Concept for Anti-CD38 mAb Efficacy

Objective: 1—To demonstrate that anti-CD38 mAb injections deplete CD38+CD8+ T cells. 2—To demonstrate that anti-CD38 mAb injections prevent the development of the clinical reaction induced by the reactivation of drug-specific CD8+ T cells.

Deliverables: In the two groups of mice injected or not with the anti-CD38 mAbs=(i) Percentage of proliferating (Ki67+) and activated (CD38+; Granulysine+ or Granzyme B+) CD8+ T cells in lymphoid organs, liver or skin (ii) Skin, liver or kidney histology. (iii) Main inflammatory cytokines/mediators in the sera (IL-1, IL-6, IL-15, TNF-α, IFNg, Granulysin, Granzyme B).

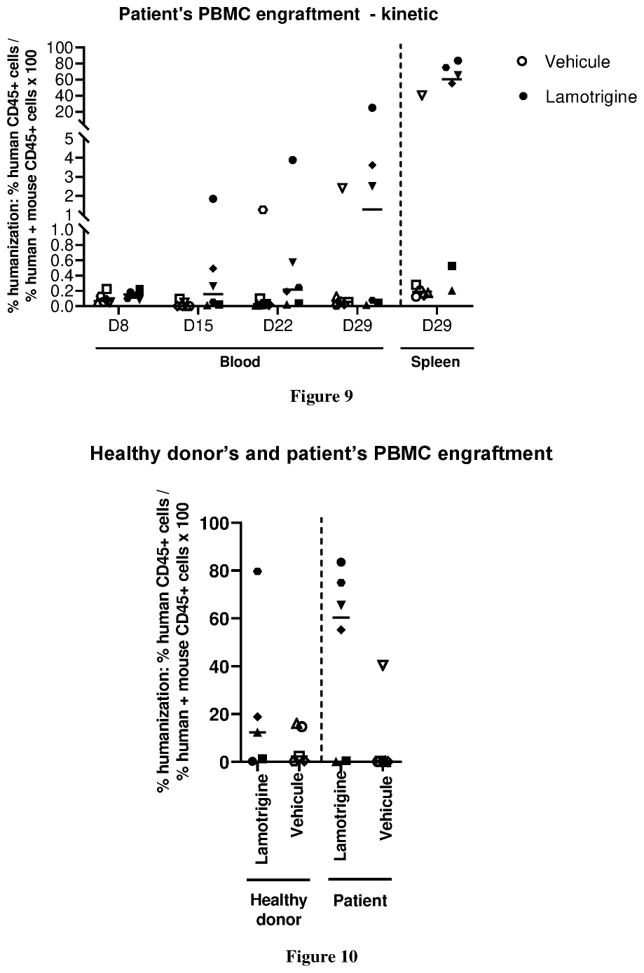

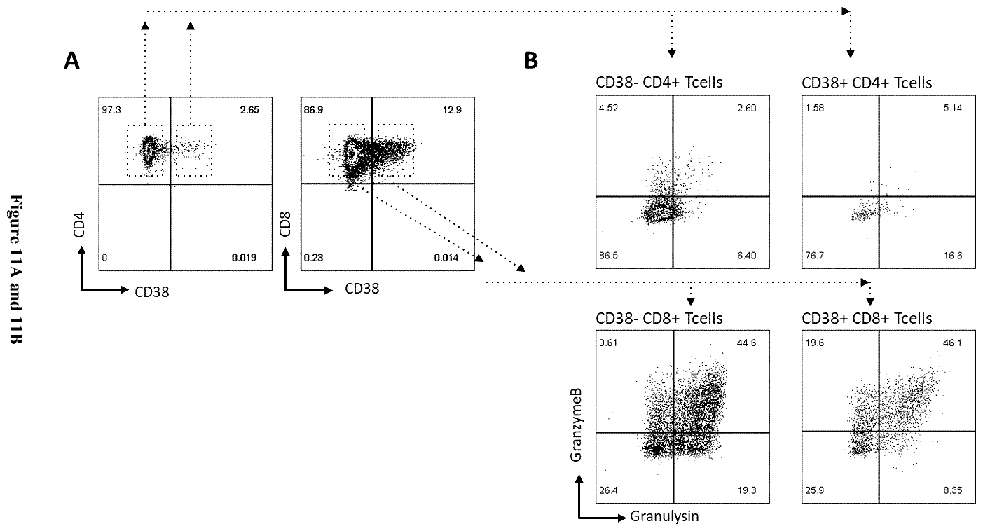

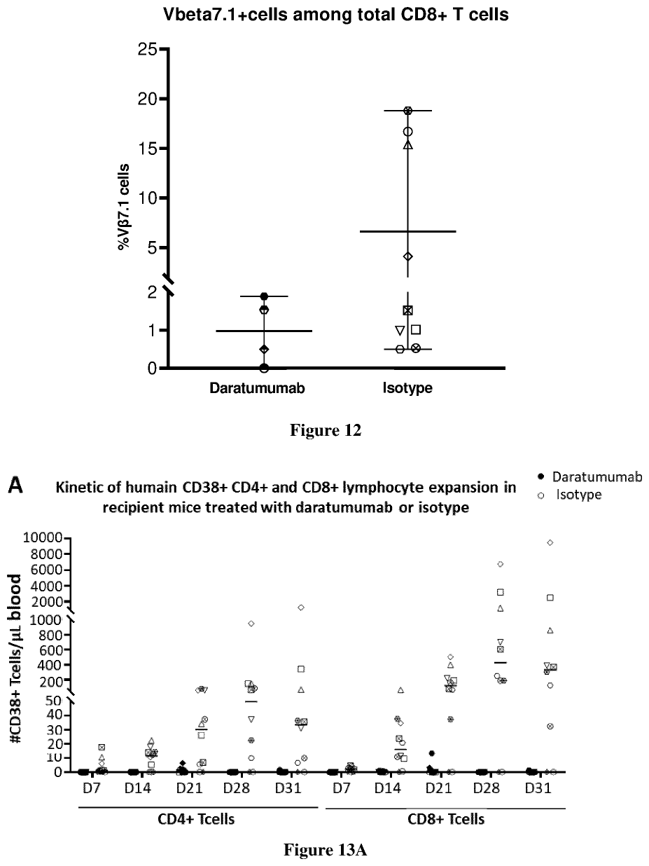

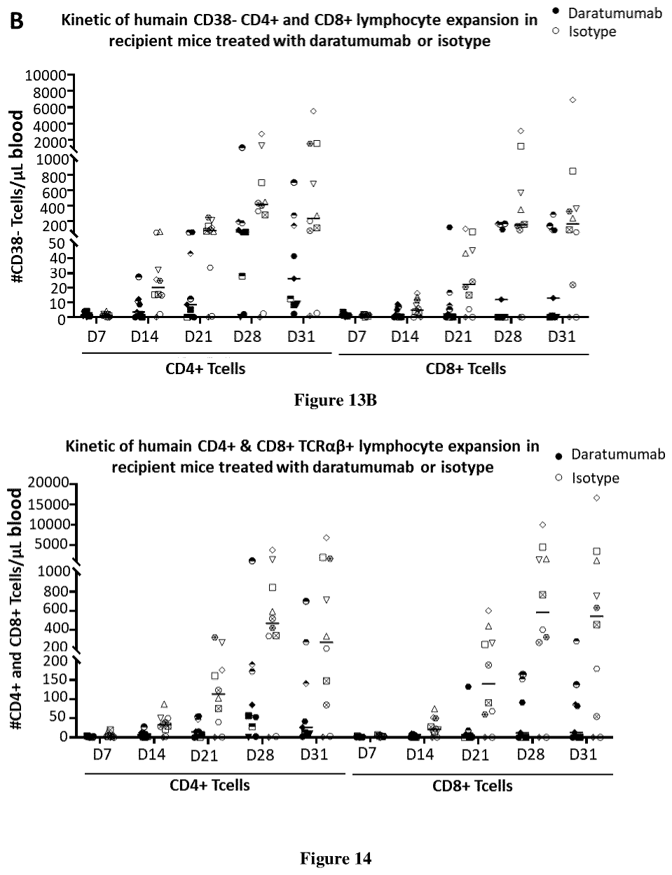

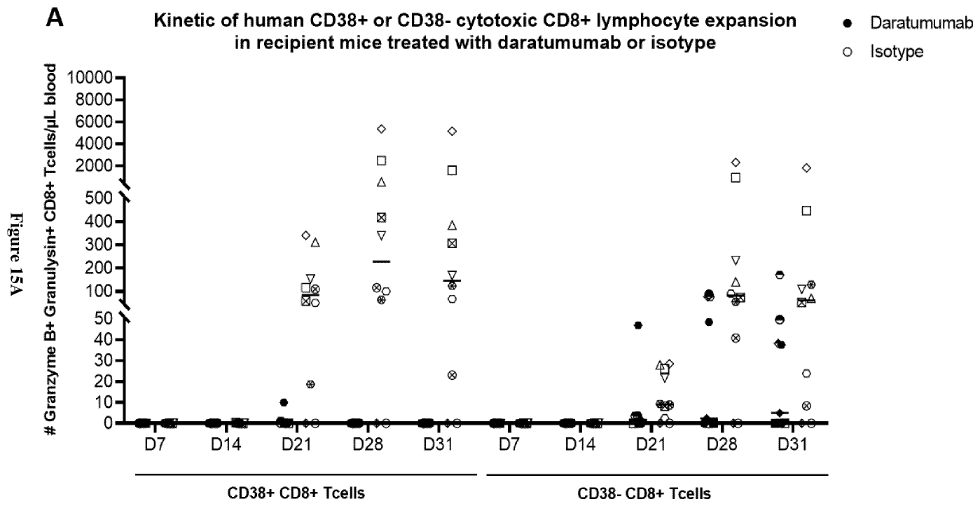

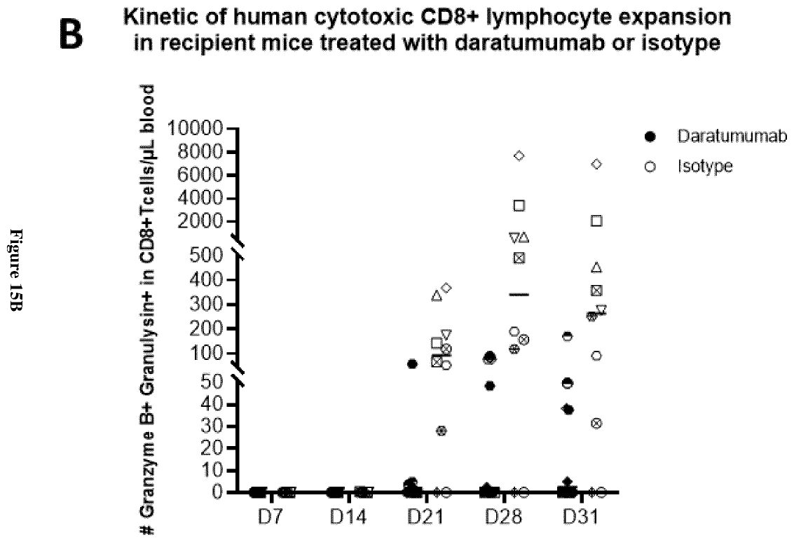

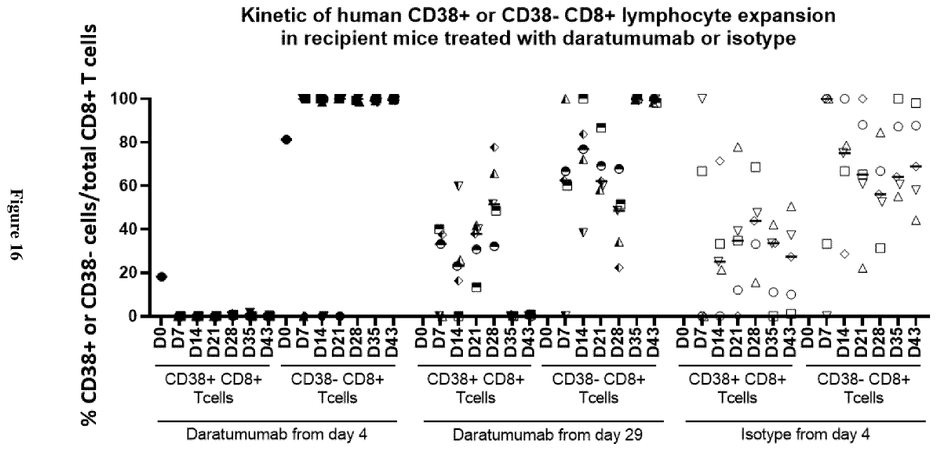

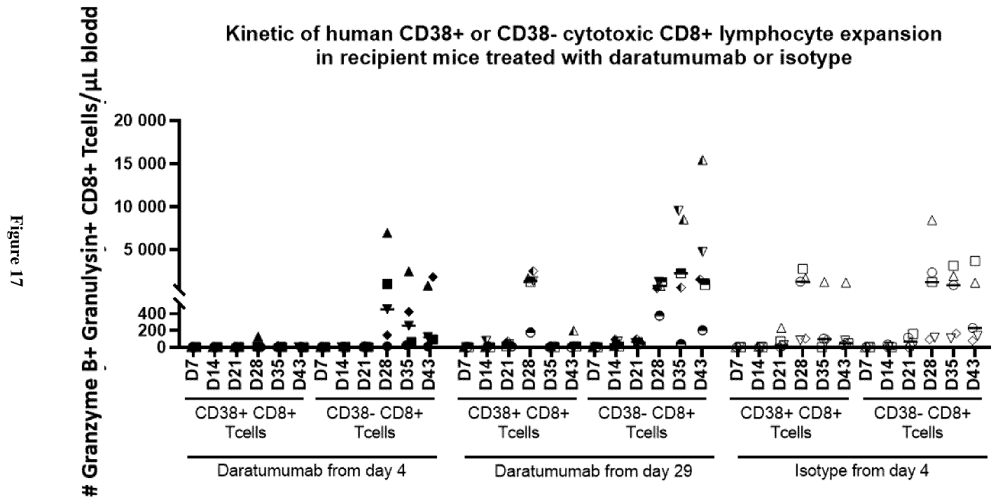

Model description: We generated a surrogate model of TEN disease in mouse, by reconstituting NSG animals with 1.10⁶ millions of peripheral blood mononuclear cells (PBMCs) collected from a TEN patient, 1 year after disease recovery. The patients' T cells were then reactivated with the offending drug delivered to animals by oral gavage. In this model, T cells progressively expanded in response to the xenogenic environment, as well as well to drug addition. Hence, as shown in FIG. 9, a progressive and strong expansion of T cells (calculated as % humanization=% human CD45+ cells/% (mouse+human) CD45+ cells×100; TCRαβ+ T cells represented >90% of human CD45+ cells in the model (data not shown)) was observed 29 days after cell transfer, both in mouse blood and spleen. A high % of humanization (>50-60%) classically correlates with the appearance of GVHD symptoms, approximately 1 month after transfer (not documented here). Interestingly, we noted that the number of animals with a high % of humanization was largely superior in the group treated with the culprit drug (here, lamotrigine) versus a control group treated with the vehicle (FIG. 9). This indicates that drug infusion accelerated T cell proliferation/expansion in the model. Comparing T cell proliferation/expansion in NGS animals reconstituted with TEN PBMCs and mice transferred with PBMCs collected from a healthy donor (HD), we next recorded a superior ability of TEN cells to proliferate in response to lamotrigine compared with HD cells (FIG. 10). Among the expanded T cells, we detected both CD4+ and CD8+ T cells (FIG. 11A), including a significant percentage of CD8+ T cells expressing CD38+ marker, as well as cytotoxic phenotype, as revealed by high Granzyme B and/or Granulysin marker expression (FIG. 11B). Importantly, we also found that some of the expanded CD8+CD38+ T cells expressed the same TCR Vbeta chain (Vbeta7.1+, FIG. 12) as the pathogenic cells collected in the skin of TEN patient at the peak of the disease. CD8+CD38+Vbeta7.1+ T cells were not found in all the engrafted animals, but this indicates a possible expansion of specific T cells upon drug infusion.

These data thus indicate that our surrogate model, in which NSG mice are reconstituted with PBMCs from TEN patients and then administered with the culprit drug, recapitulates some of the key immune parameters of the disease.

Drug evaluation: We then capitalized on this new preclinical model to make the proof of concept of the efficacy of daratumumab in depleting pathogenic cytotoxic CD8+CD38+ T cells.

The effects of daratumumab were evaluated according to two administration regimens: (i) in a “preventive” mode, i.e. daratumumab was injected by intraperitoneal route (i.p.) very early (from day 4) after PBMC transfer, when cytotoxic CD8+ T cells have not yet proliferated, and (ii) in a “curative” mode, i.e., daratamumab was injected lately, from day 29 after PBMC transfer, once cytotoxic CD8+ T cells have proliferated.

Daratumumab injections from day 4 efficiently and extensively prevented the formation of CD4+CD38+ and CD8+CD38+ T cells in the blood and the spleen of transferred NSG recipients throughout the protocol (FIG. 13). In contrast, the mAb did not avoid the progressive expansion of some T cell subsets, which do not express CD38 marker. However, by blocking the formation of CD38+ cells, which represented approximately 50% of expanded blood T cells at day 28 in isotype-treated controls (data not shown), daratumumab dramatically delayed and impaired the global T cell expansion in this model (FIG. 14). More importantly, by preventing the formation of cytotoxic CD8+CD38+Granzyme B+Granulysin+ T cell subset (FIG. 15A), daratumumab also severely impaired the accumulation of cytotoxic T cells in this model (FIG. 15B). In the same line, we recorded a strong depletion of cytotoxic cells expressing the TCRVbeta7.1+ chain in daratumumab-treated animals, but not in isotype-treated controls (FIG. 12).

Finally, by injecting daratumumab in curative mode from day 29, we demonstrated that daratumumab acutely depleted the CD38+ cells that have already expanded in the model (FIG. 16), including the cytotoxic CD8+CD38+Granzyme B+Granulysin+ T cell subset (FIG. 17).

Collectively, our results thus demonstrate the efficacy of daratumumab to deplete clonally expanded pathogenic T cells in a surrogate model of TEN disease.

Importantly, those data open new avenues for a new proof of concept study in TEN patients. After demonstrating that a single injection of daratumumab is well tolerated, and that it does not generate any side-effects (e.g. cytokine release syndrome), we will search to prove that it depletes the clonally expanded drug-specific cytotoxic CD8+CD38+ T cells. Ultimate objectives will consist to determine whether it alters the course of the disease and prevents/limits skin detachment, fatal outcome and/or invalidating sequelaes in TEN patient.

TABLE 3
Useful nucleotide and amino acid sequences for practicing the invention

| SEQ ID | |
|---|---|
| NO | Nucleotide or amino acid sequence |
| 1 (CD38 | MANCEFSPVSGDKPCCRLSRRAQLCLGVSILVLILVVVLAVVVPRWRQ |
| AA | QWSGPGTTKRFPETVLARCVKYTEIHPEMRHVDCQSVWDAFKGAFIS |
| sequence | KHPCNITEEDYQPLMKLGTQTVPCNKILLWSRIKDLAHQFTQVQRDM |
| human | FTLEDTLLGYLADDLTWCGEFNTSKINYQSCPDWRKDCSNNPVSVFW |
| isoform 1) | KTVSRRFAEAACDVVHVMLNGSRSKIFDKNSTFGSVEVHNLQPEKVQ |
| | TLEAWVIHGGREDSRDLCQDPTIKELESIISKRNIQFSCKNIYRPDKFLQ |
| | CVKNPEDSSCTSEI |
| 2 (CD38 | gcagtttcagaacccagccagcctctctcttgctgcctagcctcctgccggcctcatcttcgcccagccaacccc |
| nucleic | gcctggagccctatggccaactgcgagttcagcccggtgtccggggacaaaccctgctgccggctctctagga |
| acid | gagcccaactctgtcttggcgtcagtatcctggtcctgatcctcgtcgtggtgctcgcggtggtcgtcccgaggt |
| sequence | ggcgccagcagtggagcggtccgggcaccaccaagcgctttcccgagaccgtcctggcgcgatgcgtcaag |
| human | tacactgaaattcatcctgagatgagacatgtagactgccaaagtgtatgggatgctttcaagggtgcatttatttc |
| isoform 1) | aaaacatccttgcaacattactgaagaagactatcagccactaatgaagttgggaactcagaccgtaccttgcaa |
| | caagattcttctttggagcagaataaaagatctggcccatcagttcacacaggtccagcgggacatgttcaccct |
| | ggaggacacgctgctaggctaccttgctgatgacctcacatggtgtggtgaattcaacacttccaaaataaactat |
| | caatcttgcccagactggagaaaggactgcagcaacaaccctgtttcagtattctggaaaacggtttcccgcag |
| | gtttgcagaagctgcctgtgatgtggtccatgtgatgctcaatggatcccgcagtaaaatctttgacaaaaacagc |
| | acttttgggagtgtggaagtccataatttgcaaccagagaaggttcagacactagaggcctgggtgatacatggt |
| | ggaagagaagattccagagacttatgccaggatcccaccataaaagagctggaatcgattataagcaaaagga |
| | atattcaattttcctgcaagaatatctacagacctgacaagtttcttcagtgtgtgaaaaatcctgaggattcatcttg |
| | cacatctgagatctgagccagtcgctgtggttgttttagctccttgactccttgtggtttatgtcatcatacatgactc |
| | agcatacctgctggtgcagagctgaagattttggagggtcctccacaataaggtcaatgccagagacggaagc |
| | ctttttccccaaagtcttaaaataacttatatcatcagcatacctttattgtgatctatcaatagtcaagaaaaattattg |
| | tataagattagaatgaaaattgtatgttaagttacttcactttaattctcatgtgatccttttatgttatttatatattggtaa |
| | catcctttctattgaaaaatcaccacaccaaacctctcttattagaacaggcaagtgaagaaaagtgaatgctcaa |
| | gtttttcagaaagcattacatttccaaatgaatgaccttgttgcatgatgtatttttgtacccttcctacagatagtcaa |
| | accataaacttcatggtcatgggtcatgttggtgaaaattattctgtaggatataagctacccacgtacttggtgcttt |
| | accccaacccttccaacagtgctgtgaggttggtattatttcattttttagatgagaaaatgggagctcagagaggt |
| | tatatatttaagttggtgcaaaagtaattgcaagttttgccaccgaaaggaatggcaaaaccacaattatttttgaac |
| | caacctaataatttaccgtaagtcctacatttagtatcaagctagagactgaatttgaactcaactctgtccaactcc |
| | aaaattcatgtgctttttccttctaggcctttcataccaaactaatagtagtttatattctcttccaacaaatgcatattg |
| | gattaaattgactagaatggaatctggaatatagttcttctggatggctccaaaacacatgtttttcttcccccgtctt |
| | cctcctcctcttcatgctcagtgttttatatatgtagtatacagttaaaatatacttgttgctggtactggcagcttatatt |
| | ttctctcttttttcatggattaaccttgcttgagggctttaacaattgtattactttttcaaagaactaagctttagcttcat |
| | tgatttttttctatttaattgggttttgctcttctctttagcattggaaacatagaaatgctttctgatttctttgggtagattt |
| | acgtattcagcttcttgagatggaagtttagatcactgatccttcagcttgttttcttttttgtatacatagattttaggac |
| | gatatattttcccttgagttctgctttagctgcagctcttatgttttgatatgcctctctttattatccttcagttaaaaatat |
| | ctttcaattcattgttatataaaaatatgtgcctagtttttaacatctggagattttctagttttgaaaaaaacataagcc |
| | aggcatggtggctcacacctgtatccccagcactttgggaggccgagacgggaggatcgcctgagctcagga |
| | gtttttacaccagcctgggaataacagtgagacattatctccaaaaaaattacctgggtatggtgttgtgcacctgt |
| | agtcccagctactctggagactgaggtgggaggattgtttgagcttgggaggttgaggctgcagggagctgtg |
| | atcacaccactgcactctggcctgagtgacagattgagaccctgtctcaataaaagcaaaaataaagaaaataa |
| | accatatgtgttgaacaaaggattaataaattaatttgagactccttcagggaatgaccacaatttattgaaaatagc |
| | ctaaatgttggagtcaggcatttctggattcatattttgacatcatgctgtcatcttgaacaaaatgcctaacctttctg |
| | aacttcaacttccttgccactcaaataaggattacaaaacttaaaatgtggtaagtactaaagacgacagcaaaaa |
| | ttgagtccagcacagagcttcctaaataagcaagcactcaacagagttggttcctttcttcctcccctgcttgacaa |
| | tccagtttcccacaggagcctttgtagctgtagccaccatggtcagtccagggattcttcactagccccttctcccc |
| | tggcagacatccttgtgggagtttagtcttggctcgacatgaggatgggggtttgggaccagttctgagtgagaa |
| | tcagacttgccccaagttgccattagctccccctgcagaatgtcttcagaatcggggcccggtcagtctcctggg |
| | tgacctgctgttttcctcttaagatcctttccactttggttgctgctttcgggactcatcgagtccttgctcaacaggat |
| | accccttgaagtggctgcctgggccacatccccttccaaacaagaaatcaaaatattagaaatcaatttttgaaatt |
| | tcccctaggaagactcatttgagtgttcaagttcagagccagtggagaccttaggggagggtggtcacaaggat |
| | tttgcacagtgctttagagggtcccagggagccacagaggtggtgaggggctgggtgctcttttctccgtgcatg |
| | accttgtgtgtctatcttcattaccacaatgcctcatctctacctcctttccccctgtagttccaacgtgggtatctttg |
| | ccatctctggcccgaaggactttctgacctacatgtataaataccccctcacaatatatattacttttcctataagtga |
| | cttctctactggattactggttgctcatacacctcatattttactcgtaaatctactactccctgtctgcctactccattct |
| | catttgctgtagaaaattctcttaccatcccaactttcacccaccatcatgcttacccaaaggctgtgggaatgacc |
| | tgggccctaatgccccttttctaaattcctaaggctcaccattttcctattgtaatggttcttgaccttataatgtttgag |
| | gcaccttttcaaatatagtcctttgatttcagactgaatacttgaaaggacacacacacacatacgtaagtgcatat |
| | gactgcatacacccacacacacacacgtgcctgtatacagtcatatgatacatacacaaacacacgcacacaag |
| | cctgcatacatcatatgccaacagtggggatatgttctgagaaatgcatcattagatgattttgtcattgtgtgaaca |
| | tcatagagtgtacttacactaacctagatggtctaacctactacacacccaggctacatggtatcacctattcctcct |
| | aggctacaagcctgtacagcgtgtgtctgtactaaatgctgtgggcaattttaacctgatggtaaatgtttgtgtatc |
| | taaacatatctaaacatagaaaaggtacagtaaacatgcagtattataatcttatgagaccgtcatcatatatgtggt |
| | ccactgtttgggccatcattggctgaaaagtggttatgcgacacatgactgtatatatactttcctgttacaacaaca |
| | gtgtctctcaatccacagtaattgcagcatccagtaggtcttactttagccctgagtcaccatttgtgtcaacgtgttt |
| | agtgccatgtccacgtctctcatgtaactggcagagctatcaaatattttggcaaaacacattgtttctttggctttgc |
| | cttggtaactttctgtgccttttgtagctcttgtttggaagaagctcaacccatgtctgcacactgtgatacaagggg |
| | gacagcatcgacatcgacttacttcttggtgccttattcctccttagaacaattcctaaatctgtaacttaagtttctca |
| | ggaagattccatactgcacagaaaactgcttttgtgggtttttaaaaggcaagttgttatatgtgctggatagttttta |
| | agtatgacataaaaattgtataaagtaaaatattaaaatacacctagaatactgtataactttaagtcattttatcaac |
| | acattgctaatccagatattttcccgcagttttttttgaataacagagcaattaatttacttttactatgaagagtcatc |
| | attttagtatgtattttaagcaatccaccaagaactcagtaggcagctgagaggtgctgcccagagaagtggtgat |
| | tagcttggccttagctcacccacacaaagcacaacaggctttgaactattccctaacggggcatttattctttttttttt |
| | ttttttttgggagacggagtctcgctgtcgcccaggctagagtgcagtggcgcgatctcggctcactgcaggctc |
| | caccccctggggttcacgccattctcctgcctcagcctcccaagtagctgggactgcaggcgcccgccatctcg |
| | cccggctaattttttgtatttttagtagagacggggtttcaccgtgttagccaggatagggcatttattcttgaacttg |
| | attcagagaggcacacattaccattctctaatcagaatgcaagtagcgcaaggcggtggaaactatggaattcg |
| | gaggcaggtgatgcattgggcgagtttattaacatctgtgactctctagtttgaaatttatttgtaacagacaaaaat |
| | gaattaaacaaacaataaaagtataataaagaa |

### REFERENCES

Throughout this application, various references describe the state of the art to which this invention pertains. The disclosures of these references are hereby incorporated by reference into the present disclosure.
1. A. P. Villani et al., Nécrolyse épidermique toxique: physiopathologie et avancées thérapeutiques Rev Fr Allergol 57, 418-425 (2017).
2. T. A. Duong, L. Valeyrie-Allanore, P. Wolkenstein, O. Chosidow, Severe cutaneous adverse reactions to drugs. Lancet 390, 1996-2011 (2017).
3. C. B. Chen et al., An Updated Review of the Molecular Mechanisms in Drug Hypersensitivity. J Immunol Res 2018, 6431694 (2018).
4. W. H. Chung et al., Oxypurinol-Specific T Cells Possess Preferential TCR Clonotypes and Express Granulysin in Allopurinol-Induced Severe Cutaneous Adverse Reactions. J Invest Dermatol 135, 2237-2248 (2015).
5. A. Nassif et al., Toxic epidermal necrolysis: effector cells are drug-specific cytotoxic T cells. The Journal of allergy and clinical immunology 114, 1209-1215 (2004).
6. A. Nassif et al., Drug specific cytotoxic T-cells in the skin lesions of a patient with toxic epidermal necrolysis. J Invest Dermatol 118, 728-733 (2002).
7 J. Adam et al., Abacavir induced T cell reactivity from drug naive individuals shares features of allo-immune responses. PloS one 9, e95339 (2014).
8. W. Hoetzenecker et al., Toxic epidermal necrolysis. F1000Res 5, (2016).
9. W. J. Pichler, D. J. Naisbitt, B. K. Park, Immune pathomechanism of drug hypersensitivity reactions. The Journal of allergy and clinical immunology 127, S74-81 (2011).
10. M. Vocanson, D. J. Naisbitt, J. F. Nicolas, Current perspective of the etiopathogenesis of delayed-type, and T-cell-mediated drug-related skin diseases. The Journal of allergy and clinical immunology, (2020).
11. W. J. Pichler, O. Hausmann, Classification of Drug Hypersensitivity into Allergic, p-i, and Pseudo-Allergic Forms. International archives of allergy and immunology 171, 166-179 (2016).
12. P. T. Illing et al., Immune self-reactivity triggered by drug-modified HLA-peptide repertoire. Nature 486, 554-558 (2012).
13. D. A. Ostrov et al., Drug hypersensitivity caused by alteration of the MHC-presented self-peptide repertoire. Proceedings of the National Academy of Sciences of the United States of America 109, 9959-9964 (2012).
14. W. H. Chung et al., Granulysin is a key mediator for disseminated keratinocyte death in Stevens-Johnson syndrome and toxic epidermal necrolysis. Nature medicine 14, 1343-1350 (2008).
15 I. Viard-Leveugle et al., TNF-alpha and IFN-gamma are potential inducers of Fas-mediated keratinocyte apoptosis through activation of inducible nitric oxide synthase in toxic epidermal necrolysis. J Invest Dermatol 133, 489-498 (2013).
16. C. Schlapbach et al., NKp46+ cells express granulysin in multiple cutaneous adverse drug reactions. Allergy 66, 1469-1476 (2011).
17. E. de Araujo et al., Death ligand TRAIL, secreted by CD1a+ and CD14+ cells in blister fluids, is involved in killing keratinocytes in toxic epidermal necrolysis. Experimental dermatology 20, 107-112 (2011).
18. N. Saito et al., An annexin A1-FPR1 interaction contributes to necroptosis of keratinocytes in severe cutaneous adverse drug reactions. Science translational medicine 6, 245ra295 (2014).
19. M. Hertl et al., Predominance of epidermal CD8+ T lymphocytes in bullous cutaneous reactions caused by beta-lactam antibiotics. J Invest Dermatol 101, 794-799 (1993).
20. Y. Hari et al., T cell involvement in cutaneous drug eruptions. Clinical and experimental allergy: journal of the British Society for Allergy and Clinical Immunology 31, 1398-1408 (2001).
21. N. Yawalkar, W. J. Pichler, Pathogenesis of drug-induced exanthema. International archives of allergy and immunology 124, 336-338 (2001).
22. F. Skowron et al., Comparative histological analysis of drug-induced maculopapular exanthema and DRESS. Journal of the European Academy of Dermatology and Venereology: JEADV 30, 2085-2090 (2016).
23. S. Singh et al., Assessment of histopathological features of maculopapular viral exanthem and drug-induced exanthem. J Cutan Pathol 44, 1038-1048 (2017).
24. M. Mockenhaupt, Epidemiology of cutaneous adverse drug reactions. Allergol Select 1, 96-108 (2017).
25. Y. K. Jee, S. Kim, J. M. Lee, H. S. Park, S. H. Kim, CD8(+) T-cell activation by methazolamide causes methazolamide-induced Stevens-Johnson syndrome and toxic epidermal necrolysis. Clinical and experimental allergy: journal of the British Society for Allergy and Clinical Immunology 47, 972-974 (2017).
26. P. C. Kuechler et al., Cytotoxic mechanisms in different forms of T-cell-mediated drug allergies. Allergy 59, 613-622 (2004).
27. J. Dean et al., Annotation of pseudogenic gene segments by massively parallel sequencing of rearranged lymphocyte receptor loci. Genome Medicine 7, (2015).
28. H. G. Drexler, G. Gaedicke, J. Minowada, Isoenzyme studies in human leukemia-lymphoma cell lines—1. Carboxylic esterase. Leuk Res 9, 209-229 (1985).
29. R. Y. Pan et al., Identification of drug-specific public TCR driving severe cutaneous adverse reactions. Nat Commun 10, 3569 (2019).
30. T. M. Ko et al., Shared and restricted T-cell receptor use is crucial for carbamazepine-induced Stevens-Johnson syndrome. The Journal of allergy and clinical immunology 128, 1266-1276 e1211 (2011).
31. H. Xiong et al., Comprehensive assessment of T cell receptor beta repertoire in Stevens-Johnson syndrome/toxic epidermal necrolysis patients using high-throughput sequencing. Mol Immunol 106, 170-177 (2019).
32. J. L. Harden, D. Hamm, N. Gulati, M. A. Lowes, J. G. Krueger, Deep Sequencing of the T-cell Receptor Repertoire Demonstrates Polyclonal T-cell Infiltrates in Psoriasis. F1000Res 4, 460 (2015).
33. I. R. Kirsch et al., TCR sequencing facilitates diagnosis and identifies mature T cells as the cell of origin in CTCL. Science translational medicine 7, 308ra158 (2015).
34. S. C. Su et al., Interleukin-15 Is Associated with Severity and Mortality in Stevens-Johnson Syndrome/Toxic Epidermal Necrolysis. J Invest Dermatol 137, 1065-1073 (2017).
35. H. Azukizawa, Animal models of toxic epidermal necrolysis. Journal of Dermatology 38, 255-260 (2011).
36. R. Takahashi et al., Defective Regulatory T Cells In Patients with Severe Drug Eruptions: Timing of the Dysfunction Is Associated with the Pathological Phenotype and Outcome. Journal of immunology 182, 8071-8079 (2009).
37. N. Yoshioka et al., Disturbed balance in three subpopulations of CD4(+)Foxp3(+) regulatory T cells in Stevens-Johnson syndrome and toxic epidermal necrolysis patients. Clinical immunology 148, 89-91 (2013).
38. M. K. Jenkins, H. H. Chu, J. B. McLachlan, J. J. Moon, On the composition of the preimmune repertoire of T cells specific for Peptide-major histocompatibility complex ligands. Annual review of immunology 28, 275-294 (2010).
39. C. A. Almeida et al., Virus-specific T-cell clonotypes might contribute to drug hypersensitivity reactions through heterologous immunity. The Journal of allergy and clinical immunology, (2019).
40. S. C. Su et al., HLA Alleles and CYP2C9*3 as Predictors of Phenytoin Hypersensitivity in East Asians. Clin Pharmacol Ther 105, 476-485 (2019).
41. S. Deaglio et al., Human CD38 (ADP-ribosyl cyclase) is a counter-receptor of CD31, an Ig superfamily member. Journal of immunology 160, 395-402 (1998).
42. R. J. Betts et al., Contact sensitizers trigger human CD1-autoreactive T-cell responses. Eur J Immunol 47, 1171-1180 (2017).
43. S. H. Kardaun et al., Variability in the clinical pattern of cutaneous side-effects of drugs with systemic symptoms: does a DRESS syndrome really exist? The British journal of dermatology 156, 609-611 (2007).
44. A. Auquier-Dunant et al., Correlations between clinical patterns and causes of erythema multiforme majus, Stevens-Johnson syndrome, and toxic epidermal necrolysis: results of an international prospective study. Arch Dermatol 138, 1019-1024 (2002).
45. B. Sassolas et al., ALDEN, an algorithm for assessment of drug causality in Stevens-Johnson Syndrome and toxic epidermal necrolysis: comparison with case-control analysis. Clin Pharmacol Ther 88, 60-68 (2010).
46. S. Bastuji-Garin et al., SCORTEN: a severity-of-illness score for toxic epidermal necrolysis. J Invest Dermatol 115, 149-153 (2000).
47. R. Finck et al., Normalization of mass cytometry data with bead standards. Cytometry A 83, 483-494 (2013).
48. S. Van Gassen et al., FlowSOM: Using self-organizing maps for visualization and interpretation of cytometry data. Cytometry A 87, 636-645 (2015).
49. R. V. Bruggner, B. Bodenmiller, D. L. Dill, R. J. Tibshirani, G. P. Nolan, Automated identification of stratifying signatures in cellular subpopulations. Proceedings of the National Academy of Sciences of the United States of America 111, E2770-2777 (2014).
50. H. G. Drexler, G. Gaedicke, J. Minowada, Isoenzyme Studies in Human Leukemia-Lymphoma Cell-Lines 0.1. Carboxylic Esterase. Leukemia Res 9, 209-& (1985).
51. D. Yerly et al., Design, expression, and processing of epitomized hepatitis C virus-encoded CTL epitopes. Journal of immunology 181, 6361-6370 (2008).
52. C. Segouffin-Cariou, G. Farjot, A. Sergeant, H. Gruffat, Characterization of the epstein-barr virus BRRF1 gene, located between early genes BZLF1 and BRLF1. J Gen Virol 81, 1791-1799 (2000).
53. D. Mauri-Hellweg et al., Activation of drug-specific CD4+ and CD8+ T cells in individuals allergic to sulfonamides, phenytoin, and carbamazepine. Journal of immunology 155, 462-472 (1995).

1. A method for assessing a subject's risk of having or developing Toxic Epidermal Necrolysis and treating the subject, comprising i) determining in a sample obtained from the subject the level of T lymphocytes having cell surface expression of CD8+CD45RA−CCR7−CD38+ markers, and ii) administering a CD38 inhibitor to a subject identified as having a level of T lymphocytes having cell surface expression of CD8+CD45RA−CCR7−CD38+ markers that is higher than a corresponding reference value.

2. The method according to claim 1, wherein the sample is a blood sample or immune primary cells or a blister sample or a skin sample.

3. The method according to claim 2, wherein the immune primary cells are selected from the group consisting of PBMC, WBC and T lymphocytes.

4. A method for monitoring and treating a Toxic Epidermal Necrolysis comprising i) determining the level of a population of T lymphocytes having cell surface expression of CD8+CD45RA−CCR7−CD38+ markers in a sample obtained from the subject at a first specific time of the disease, ii) determining the level of a population of T lymphocytes having cell surface expression of CD8+CD45RA−CCR7−CD38+ markers in a sample obtained from the subject at a second specific time of the disease, and iii) administering a CD38 inhibitor to a subject identified as having a level determined at step ii) that is higher than the level determined at step i).

5. An in vitro method for monitoring the treatment of Toxic Epidermal Necrolysis comprising the steps of i) determining the level of a population of T lymphocytes having cell surface expression of CD8+CD45RA−CCR7−CD38+ in a sample obtained from the subject before the treatment, ii) determining the level of a population of T lymphocytes having cell surface expression of CD8+CD45RA−CCR7−CD38+ markers in a sample obtained from the subject after the treatment”, iii) comparing the level determined at step i) with the level determined at step ii) and iv) concluding that the treatment is efficient when the level determined at step ii) is lower than the level determined at step i).

6. The in vitro method for monitoring according to claim 4, wherein the sample is a blood sample or immune primary cells or blister sample or skin sample.

7. The in vitro method for monitoring according to claim 4, wherein the immune primary cells selected from the group consisting of PBMC, WBC and T lymphocytes.

8. The method according to claim 1, wherein the level of the population of T lymphocytes having cell surface expression of CD8+CD45RA−CCR7−CD38+ is determined by clonal expansion of said population.

9. A method of preventing or treating a Toxic Epidermal Necrolysis in a subject in need thereof, comprising,
administering to the subject a therapeutically effective amount of a CD38 inhibitor.

10. The method according to claim 8 wherein the CD38 inhibitors is selected from:
a) an inhibitor of CD38 activity
and/or
b) an inhibitor of CD38 gene expression.

11. The method according to claim 10 wherein said inhibitor of CD38 activity is a small organic molecule, an antibody, a CAR T cell or an aptamer.

12. The method according to claim 11, wherein the antibody is selected from the group consisting Daratumumab, Isatuximab, MOR202, TAK-079, TAK-169, AMG424 or GBR 1342.

13. The method according to claim 10 wherein the inhibitor of CD38 gene expression is an antisense oligonucleotide, a nuclease, siRNA, shRNA, or ribozyme nucleic acid sequence.

14. The method according to claim 9, wherein the subject is identified having a high level of T lymphocytes CD8+CD45RA−CCR7−CD38+ in a biological sample, wherein the level by the methods of claim 1.

15. The method according to claim 14, wherein the biological sample is a blood sample or immune primary cells or a skin sample.

16. (canceled)


SEQUENCE LISTING (2 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - DNA, 5620 bp, Homo sapiens
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa